(19) United States
(12) Patent Application Publication
(10) Pub. No.: US 2023/0391873 A1
(43) Pub. Date: Dec. 7, 2023

(54) METHODS FOR TREATING MISMATCH REPAIR DEFICIENT LOCALLY ADVANCED RECTAL CANCER USING DOSTARLIMAB

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventors: Luis DIAZ, New York, NY (US); Andrea CERCEK, New York, NY (US)

(21) Appl. No.: 18/325,912

(22) Filed: May 30, 2023

Related U.S. Application Data
(60) Provisional application No. 63/348,139, filed on Jun. 2, 2022.

(51) Int. Cl. C07K 16/28; A61P 35/00

(52) U.S. Cl. CPC C07K 16/2818; A61P 35/00; A61K 2039/545

(57) ABSTRACT

The present disclosure provides compositions comprising dostarlimab and methods of using the same to treat mismatch repair deficient (MMRd) rectal cancer (e.g., locally advanced rectal cancer).

### CROSS-REFERENCE TO RELATED APPLICATIONS

This application claims the benefit of and priority to U.S. Provisional Patent Application No. 63/348,139, filed Jun. 2, 2022, the entire contents of which are incorporated herein by reference.

### SEQUENCE LISTING

The instant application contains a Sequence Listing which has been submitted electronically in XML format and is hereby incorporated by reference in its entirety. Said XML copy, created on Jun. 9, 2023, is named 115872-2785_SL.xml and is 26,670 bytes in size.

### TECHNICAL FIELD

The present technology relates generally to compositions comprising dostarlimab and methods of using the same to treat mismatch repair deficient (MMRd) rectal cancer (e.g., locally advanced rectal cancer).

### BACKGROUND

The following description of the background of the present technology is provided simply as an aid in understanding the present technology and is not admitted to describe or constitute prior art to the present technology.

Locally advanced rectal cancer is managed with multimodal therapy including chemotherapy, radiation, and surgery. Current evidence supports a strategy of neoadjuvant therapy, in which induction chemotherapy with a fluoropyrimidine in combination with oxaliplatin is followed by chemoradiation to the rectum before surgery.¹⁻³ This approach is effective in achieving pathologic complete response (pCR) in up to a quarter of patients but associated with significant morbidity and toxicity including bowel, urinary, and sexual dysfunction, infertility, and altered quality of life in a substantial proportion of patients.⁴⁻⁸ In patients requiring surgery, resection of the rectum is life altering and often requires a permanent diverting colostomy.⁸⁻¹⁰ Due to the morbidity of surgery and the high frequency of pCR, interest in organ-sparing nonoperative management is increasing. Using clinical complete response (cCR) achieved with neoadjuvant treatment as a surrogate for pCR provides patients with a nonoperative option that results in survival comparable to that of patients undergoing surgical resection.¹¹⁻¹⁵

Approximately 5-10% of rectal adenocarcinomas are mismatch repair deficient (MMRd) and these tumors have been shown to respond poorly to standard chemotherapy regimens, including neoadjuvant chemotherapy in locally advanced rectal cancer.¹⁶⁻¹⁸ Immune checkpoint blockade therapy has been shown to be effective in the first line and treatment refractory setting in MMRd metastatic colorectal cancer with objective response rates of about 33-55%, significant durability of response and prolonged overall survival.¹⁹⁻²²

Accordingly, there is an urgent need for therapeutic compositions and methods that achieve high objective response rates in MMRd locally advanced rectal cancer.

### SUMMARY OF THE PRESENT TECHNOLOGY

In one aspect, the present disclosure provides a method for treating mismatch repair deficient (MMRd) rectal cancer in a patient in need thereof comprising administering to the patient an effective amount of an anti-PD1 antibody or an antigen binding fragment thereof, wherein the anti-PD1 antibody or antigen binding fragment comprises a heavy chain immunoglobulin variable domain (V_{H}) and a light chain immunoglobulin variable domain (V_{L}), wherein the V_{H} comprises a V_{H}-CDR1 sequence of SEQ ID NO: 5, a V_{H}-CDR2 sequence of SEQ ID NO: 6, and a V_{H}-CDR3 sequence of SEQ ID NO: 7 and the V_{L} comprises a V_{L}-CDR1 sequence of SEQ ID NO: 8, a V_{L}—CDR2 sequence of SEQ ID NO: 9, and a V_{L}-CDR3 sequence of SEQ ID NO: 10, and wherein the patient has not received a prior cancer therapy. The antigen binding fragment may be selected from the group consisting of Fab, F(ab′)2, Fab′, scFv, and Fv. Examples of prior cancer therapy include immunotherapy, chemotherapy, or radiation. In certain embodiments, the chemotherapy comprises one or more of fluoropyrimidine, leucovorin calcium (folinic acid), fluorouracil, capecitabine, and oxaliplatin.

In some embodiments, the V_{H} comprises the sequence of SEQ ID NO: 3 and the V_{L} comprises the sequence of SEQ ID NO: 1. Additionally or alternatively, in some embodiments, the anti-PD-1 antibody or antigen binding fragment comprises a heavy chain (HC) amino acid sequence comprising SEQ ID NO: 4 and a light chain (LC) amino acid sequence comprising SEQ ID NO: 2. The MMRd rectal cancer may be stage II or stage III. In other embodiments, the MMRd rectal cancer is node-positive (e.g., spread to lymph nodes) or node-negative. In any and all embodiments of the methods disclosed herein, the MMRd rectal cancer has a tumor stage selected from the group consisting of T1, T2, T3 and T4.

Additionally or alternatively, in some embodiments of the methods disclosed herein, the MMRd rectal cancer is locally advanced rectal cancer. The MMRd rectal cancer may comprise a deficiency in one or more of MLH1, MSH2, MSH6 and PMS2. In any of the preceding embodiments of the methods disclosed herein, MMR deficiency of the MMRd rectal cancer is determined by immunohistochemistry. Additionally or alternatively, in certain embodiments, the MMRd rectal cancer comprises a somatic MMR mutation selected from the group consisting of MSH2 c.1165C>T, MSH2 c.1204C>T, MSH2 c.1061delA, MSH2 c.1650dupA, MSH2 c.363dupT, MSH2 c.1413_1420delACCT TCAT, MSH6 c.2319_2320delCC, MSH6 c.2319_2337delinsTA, MSH6 c2323_2337delAAGCAATGGCTTTGT (SEQ ID NO: 22), MSH6 c.643G>A, MLH1 c.469delT, and MLH1 c.1420_1426delCGGGAAG.

In any and all embodiments of the methods disclosed herein, the patient is diagnosed with Lynch Syndrome. The patient may exhibit rectal bleeding, constipation, and/or abdominal pain prior to administration of the anti-PD1 antibody or antigen binding fragment. In some embodiments, the patient comprises a germline pathogenic variant selected from the group consisting of MSH2 c.687delA, MSH2 c.8942+3A>T, MSH2 c.942+3A>T, MSH6 c.1969delC, MSH2 c.1784T>G, PMS2 c.2500_2501delinsG, MLH1 c.1489dupC, and MSH6 c.3476dupA. Additionally or alternatively, in some embodiments, the patient does not comprise a BRAF V600E mutation and/or comprises tumors having a tumor mutation burden (TMB) ranging from of 30-95 mutations per Megabase. In certain embodiments, the TMB is about 30-35, about 35-40, about 40-45, about 45-50, about 50-55, about 55-60, about 60-65, about 65-70, about 70-75, about 75-80, about 80-85, about 85-90, or about 90-95 mutations per Megabase.

Additionally or alternatively, in some embodiments, the anti-PD1 antibody or antigen binding fragment is administered intravenously, intramuscularly, intraperitoneally, subcutaneously, rectally, parenterally, or intradermally. In any of the preceding embodiments of the methods disclosed herein, the anti-PD1 antibody or antigen binding fragment is administered once per every two weeks, once per every three weeks, or once a month. In certain embodiments, the anti-PD1 antibody or antigen binding fragment is administered for at least 2 months, at least 3 months, at least 4 months, at least 5 months, at least 6 months, at least 7 months, at least 8 months, at least 9 months, at least 10 months, at least 11 months, at least 1 year, or at least 2 or more years. In any and all embodiments of the methods disclosed herein, the patient exhibits endoscopic complete response (CR) and/or radiographic CR after administration of the anti-PD1 antibody or antigen binding fragment.

### BRIEF DESCRIPTION OF THE DRAWINGS

FIGS. 1A-1B. Evolution of endoscopic and radiographic response in patients treated with dostarlimab. Shown are the results of endoscopic evaluations, T2-weighted MRI of the rectum, and ¹⁸F-fluorodeoxyglucose-positron-emission tomography (FDG-PET) for two representative patients at baseline and at 3 months and 6 months. FIG. 1A: Patient 2 shows an endoscopic complete response and a near-complete response on T2-weighted rectal MRI at 3 months and a clinical complete response at 6 months. FIG. 1B: Patient 9 shows an endoscopic complete response and a radiographic complete response at 3 months. Arrows identify the tumor at each time point. DWI rectal MRI images are available in FIG. 5.

FIGS. 2A-2D. Biopsy Specimens of the Rectum before and after PD-1 Blockade and Viable Tumor Cell Content. FIGS. 2A-2B: Hematoxylin and eosin staining of representative biopsy specimens obtained at baseline show examples of viable tumor cells (FIG. 2A, asterisk) surrounded by necrosis (FIG. 2A, arrow) and extensive necrosis and inflammation with scant viable tumor cells (FIG. 2B). FIG. 2C: Staining of representative biopsy specimens obtained at 3 months after initiation of treatment shows the presence of acellular residual mucin pools (arrow). FIG. 2D shows the estimated percentages of viable tumor cells on pathological examination of specimens obtained before treatment (baseline), during treatment (6 weeks through 6 months), and during follow-up in 10 patients. PD-1 denotes programmed death 1.

FIGS. 3A-3D. Immune Contexture Changes after PD-1 Blockade in Rectal Tumors and Mucosa. FIG. 3A: Representative multicolor fluorescence images show tumor and normal epithelial cells that are positive for cytokeratin, programmed death ligand 1 (PD-L1) protein, CD8+T lymphocytes, and CD20+B lymphocytes in rectal biopsy samples that were obtained at baseline and after 6 weeks, 3 months, and 6 months of PD-1 blockade. FIGS. 3B-3D: Changes in the levels of PD-L1 protein (FIG. 3B), CD8+T lymphocytes (FIG. 3C), and CD20+B lymphocytes (FIG. 3D) that were selectively measured in the total tissue areas, in the cytokeratin-positive tumor and normal epithelial cell areas (labeled as “epithelial”), and in the cytokeratin-negative stromal tissue compartment are shown across multiple biopsy time points before and after initiation of treatment. Also shown are the mean factor change ±SE (I bars) of each marker relative to baseline levels in each patient for whom tissue samples could be evaluated. In addition, mean±SE (T bars) quantitative immunofluorescence (QIF) scores of PD-L1 protein (FIG. 3B), CD8+T lymphocytes (FIG. 3C), and CD20+B lymphocytes (FIG. 3D) that were selectively measured in tumor and normal epithelial cells and in stromal cells in biopsy samples obtained at baseline, during treatment (week 6 through month 6), and after treatment are shown. The number of individual samples included in each group is indicated above each bar. P values were calculated with the use of the Mann-Whitney test. DAPI denotes 4′,6-diamidine-2-phenylindole.

FIG. 4 shows the experimental design of the clinical trial described herein.

FIGS. 5A-5C show DWI Rectal MRI images of the patients. DWI-MRI was utilized to further interrogate the appearance of scar on T2W images. A bright signal focally (“restricted diffusion”) indicates tumor. FIGS. 5A-5C: Patient A corresponds to Pt ID #2 in FIG. 1 and Patient B corresponds to Pt ID #9 in FIGS. 1A-1B at the same time points. For both patients, the red arrow in FIG. 5A indicates tumor with extensive DWI bright signal (note bright dots correspond to lymph nodes), with disappearance of tumor by FIG. 5C in both patients and the presence of a uniform DWI signal corresponding to a normal rectal wall.

FIGS. 6A-6B show cumulative incidence of Median time to endoscopic and radiographic complete response among patients who completed treatment (n=12). FIG. 6A: Cumulative incidence of endoscopic complete response. Median time to endoscopic complete response was 6.1 months. Five patients (ID 2, 8, 9, 10, 11) achieved an endoscopic CR at the 3-month assessment. FIG. 6B: Cumulative incidence of radiographic complete response. Median time to radiographic complete response was 6.1 months. Two patients (ID 3, 9) achieved a radiographic CR at the 3-month assessment. CR: complete response; nCR: near complete response; PR: partial response.

FIG. 7 shows individual patient longitudinal data (n=14). Depiction of individual longitudinal endoscopic, pathologic, rectal MRI and PET data at baseline, 6 weeks, 3 months, 6 months and in follow up.

FIG. 8 shows rectal tumor SUVmax (n=12). Each line represents an individual patient rectal tumor SUVmax over time at baseline (0 months), 3-month assessment and 6-month assessment. All patients with at least two FDG PET scans, one at baseline and one at 3 months, demonstrated a marked decrease in rectal tumor SUVmax (ID 1-13). Three patients had only the baseline FDGPET available (ID 14-16). When rectal SUV was determined to be normal with only physiologic uptake and exact SUV was not measured, liver SUVmax was used for the purposes of this analysis. Two SUVmax values were not available due to diffuse FDG uptake in the rectum consistent with inflammation (ID 5 and 11, 6-month assessment). SUVmax: maximum standardized uptake value; FDG PET: fluorodeoxyglucose positron emission tomography.

FIG. 9 shows clinical symptoms.

FIG. 10 shows adverse events.

FIGS. 11-12 show expanded individual data for each patient. FIG. 11 discloses SEQ ID NO: 22.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001)Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

Neoadjuvant immunotherapy has been tested in several solid tumors,²⁸⁻³¹ including those known to be sensitive to checkpoint blockade in the metastatic setting, such as non-small cell lung cancer (NSCLC), urothelial carcinoma, and melanoma. However, none have demonstrated such profound activity as observed in MMRd rectal cancer patients receiving dostarlimab as described herein. For example, in NSCLC, a study of 2 doses of PD-1 blockade demonstrated a response rate of 10% and the experience in melanoma demonstrated a response rate of 52% with immunotherapy alone.^{29,33} A pilot study in which patients with MMRd colon cancers received a single dose of ipilimumab and two doses of nivolumab before surgery demonstrated an ORR of 50%.³⁴ Another study in early-stage treatment-refractory MMRd colorectal cancer, treated with 3 months of either the PD-1 blocking antibody, toripalimab plus celecoxib or the toripalimab monotherapy achieved a radiographic ORR of ˜55%.³⁵ In the treatment-naïve metastatic setting, the radiographic complete response rate of MMRd colorectal tumors was 11.1% despite comparable baseline molecular features like elevated, PD-L1 and TILs.³⁶ In all these studies, however, all patients proceeded to surgical resection, thereby incurring the long-term morbidity associated with having undergone that procedure.

As described in the Examples herein, the elimination of tumors following six months of dostarlimab therapy enabled omission of both chemoradiation and surgery. Because surgery and radiation have permanent effects on fertility, sexual health, bowel, and bladder function,^{4-8,26} the implications for quality of life are substantial, especially in those where standard treatment would impact childbearing potential. As the incidence of rectal cancer is rising in young adults, the methods disclosed herein are clinically significant.²⁷

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, or topically. Administration includes self-administration and the administration by another.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. As used herein, “antibodies” (includes intact immunoglobulins) and “antigen binding fragments” specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴ M⁻¹ greater or at least 10⁵ M⁻¹ greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

More particularly, antibody refers to a polypeptide ligand comprising at least a light chain immunoglobulin variable region or heavy chain immunoglobulin variable region which specifically recognizes and binds an epitope of an antigen. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody. Typically, an immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopt a p-sheet conformation and the CDRs form loops which connect, and in some cases form part of, the j-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. An antibody that binds PD-1 protein will have a specific V_{H} region and the V_{L} region sequence, and thus specific CDR sequences. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs). “Immunoglobulin-related compositions” as used herein, refers to antibodies (including monoclonal antibodies, polyclonal antibodies, humanized antibodies, chimeric antibodies, recombinant antibodies, multi-specific antibodies, bispecific antibodies, etc.,) as well as antibody fragments. An antibody or antigen binding fragment thereof specifically binds to an antigen.

As used herein, the term “antibody-related polypeptide” means antigen-binding antibody fragments, including single-chain antibodies, that can comprise the variable region(s) alone, or in combination, with all or part of the following polypeptide elements: hinge region, CH₁, CH₂, and CH₃ domains of an antibody molecule. Also included in the technology are any combinations of variable region(s) and hinge region, CH₁, CH₂, and CH₃ domains. Antibody-related molecules useful in the present methods, e.g., but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Examples include: (i) a Fab fragment, a monovalent fragment consisting of the V_{L}, V_{H}, C_{L} and CH₁ domains; (ii) a F(ab′)₂ fragment, a bivalent fragment comprising two Fab fragments linked by a disulfide bridge at the hinge region; (iii) a Fd fragment consisting of the V_{H} and CH₁ domains; (iv) a Fv fragment consisting of the V_{L} and V_{H} domains of a single arm of an antibody, (v) a dAb fragment (Ward et al., Nature 341: 544-546, 1989), which consists of a V_{H} domain; and (vi) an isolated complementarity determining region (CDR). As such “antibody fragments” or “antigen binding fragments” can comprise a portion of a full length antibody, generally the antigen binding or variable region thereof. Examples of antibody fragments or antigen binding fragments include Fab, Fab′, F(ab′)₂, and Fv fragments; diabodies; linear antibodies; single-chain antibody molecules; and multi-specific antibodies formed from antibody fragments.

As used herein, the term “diabodies” refers to small antibody fragments with two antigen-binding sites, which fragments comprise a heavy-chain variable domain (V_{H}) connected to a light-chain variable domain (V_{L}) in the same polypeptide chain (V_{H} V_{L}). By using a linker that is too short to allow pairing between the two domains on the same chain, the domains are forced to pair with the complementary domains of another chain and create two antigen binding sites. Diabodies are described more fully in, e.g., EP 404,097; WO 93/11161; and Hollinger et al., Proc. Natl. Acad. Sci. USA, 90: 6444-6448 (1993).

As used herein, the terms “single-chain antibodies” or “single-chain Fv (scFv)” refer to an antibody fusion molecule of the two domains of the Fv fragment, V_{L} and V_{H}. Single-chain antibody molecules may comprise a polymer with a number of individual molecules, for example, dimer, trimer or other polymers. Furthermore, although the two domains of the Fᵥ fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single-chain Fᵥ (scFᵥ)). Bird et al. (1988) Science 242:423-426 and Huston et al. (1988) Proc. Natl. Acad Sci. USA 85:5879-5883. Such single-chain antibodies can be prepared by recombinant techniques or enzymatic or chemical cleavage of intact antibodies.

Any of the above-noted antibody fragments are obtained using conventional techniques known to those of skill in the art, and the fragments are screened for binding specificity and neutralization activity in the same manner as are intact antibodies.

The term “antigen binding fragment” refers to a fragment of the whole immunoglobulin structure which possesses a part of a polypeptide responsible for binding to antigen. Examples of the antigen binding fragment useful in the present technology include scFv, (scFv)₂, scFvFc, Fab, Fab′ and F(ab′)₂, but are not limited thereto. Any of the above-noted antibody fragments are obtained using conventional techniques known to those of skill in the art, and the fragments are screened for binding specificity and neutralization activity in the same manner as are intact antibodies.

As used herein, an “antigen” refers to a molecule to which an antibody (or antigen binding fragment thereof) can selectively bind. The target antigen may be a protein, carbohydrate, nucleic acid, lipid, hapten, or other naturally occurring or synthetic compound. In some embodiments, the target antigen may be a polypeptide (e.g., a PD-1 polypeptide). An antigen may also be administered to an animal to generate an immune response in the animal.

By “binding affinity” is meant the strength of the total noncovalent interactions between a single binding site of a molecule (e.g., an antibody) and its binding partner (e.g., an antigen or antigenic peptide). The affinity of a molecule X for its partner Y can generally be represented by the dissociation constant (K_{D}). Affinity can be measured by standard methods known in the art, including those described herein. A low-affinity complex contains an antibody that generally tends to dissociate readily from the antigen, whereas a high-affinity complex contains an antibody that generally tends to remain bound to the antigen for a longer duration.

As used herein, the term “biological sample” means sample material derived from living cells. Biological samples may include tissues, cells, protein or membrane extracts of cells, and biological fluids (e.g., ascites fluid or cerebrospinal fluid (CSF)) isolated from a subject, as well as tissues, cells and fluids present within a subject. Biological samples of the present technology include, but are not limited to, samples taken from eye, breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, thymus, blood, hair, buccal, skin, serum, plasma, CSF, semen, prostate fluid, seminal fluid, urine, feces, sweat, saliva, sputum, mucus, bone marrow, lymph, and tears. Biological samples can also be obtained from biopsies of internal organs. Biological samples can be obtained from subjects for diagnosis or research or can be obtained from non-diseased individuals, as controls or for basic research. Samples may be obtained by standard methods including, e.g., venous puncture and surgical biopsy. In certain embodiments, the biological sample is a tissue sample obtained by needle biopsy.

As used herein, the term “CDR-grafted antibody” means an antibody in which at least one CDR of an “acceptor” antibody is replaced by a CDR “graft” from a “donor” antibody possessing a desirable antigen specificity.

As used herein, the term “chimeric antibody” means an antibody in which the Fc constant region of a monoclonal antibody from one species (e.g., a mouse Fc constant region) is replaced, using recombinant DNA techniques, with an Fc constant region from an antibody of another species (e.g., a human Fc constant region). See generally, Robinson et al., PCT/US86/02269; Akira et al., European Patent Application 184,187; Taniguchi, European Patent Application 171,496; Morrison et al., European Patent Application 173,494; Neuberger et al., WO 86/01533; Cabilly et al. U.S. Pat. No. 4,816,567; Cabilly et al., European Patent Application 0125,023; Better et al., Science 240: 1041-1043, 1988; Liu et al., Proc. Natl. Acad. Sci. USA 84: 3439-3443, 1987; Liu et al., J. Immunol 139: 3521-3526, 1987; Sun et al., Proc. Natl. Acad. Sci. USA 84: 214-218, 1987; Nishimura et al., Cancer Res 47: 999-1005, 1987; Wood et al., Nature 314: 446-449, 1885; and Shaw et al., J. Natl. Cancer Inst. 80: 1553-1559, 1988.

As used herein, the term “conjugated” refers to the association of two molecules by any method known to those in the art. Suitable types of associations include chemical bonds and physical bonds. Chemical bonds include, for example, covalent bonds and coordinate bonds. Physical bonds include, for instance, hydrogen bonds, dipolar interactions, van der Waal forces, electrostatic interactions, hydrophobic interactions and aromatic stacking.

As used herein, the term “consensus FR” means a framework (FR) antibody region in a consensus immunoglobulin sequence. The FR regions of an antibody do not contact the antigen.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, the term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and non-conformational epitopes are distinguished in that the binding to the former but not the latter is lost in the presence of denaturing solvents. In some embodiments, an “epitope” of the PD-1 protein is a region of the protein to which the anti-PD-1 antibodies of the present technology specifically bind. In some embodiments, the epitope is a conformational epitope or a non-conformational epitope.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “gene” means a segment of DNA that contains all the information for the regulated biosynthesis of an RNA product, including promoters, exons, introns, and other untranslated regions that control expression.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. A polynucleotide or polynucleotide region (or a polypeptide or polypeptide region) has a certain percentage (for example, at least 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art. In some embodiments, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the National Center for Biotechnology Information. Biologically equivalent polynucleotides are those having the specified percent homology and encoding a polypeptide having the same or similar biological activity. Two sequences are deemed “unrelated” or “non-homologous” if they share less than 40% identity, or less than 25% identity, with each other.

As used herein, “humanized” forms of non-human (e.g., murine) antibodies are chimeric antibodies which contain minimal sequence derived from non-human immunoglobulin. For the most part, humanized antibodies are human immunoglobulins in which hypervariable region residues of the recipient are replaced by hypervariable region residues from a non-human species (donor antibody) such as mouse, rat, rabbit or nonhuman primate having the desired specificity, affinity, and capacity. In some embodiments, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance such as binding affinity. Generally, the humanized antibody will comprise substantially all of at least one, and typically two, variable domains (e.g., Fab, Fab′, F(ab′)₂, or Fv), in which all or substantially all of the hypervariable loops correspond to those of a non-human immunoglobulin and all or substantially all of the FR regions are those of a human immunoglobulin consensus FR sequence although the FR regions may include one or more amino acid substitutions that improve binding affinity. The number of these amino acid substitutions in the FR are typically no more than 6 in the H chain, and in the L chain, no more than 3. The humanized antibody optionally may also comprise at least a portion of an immunoglobulin constant region (Fc), typically that of a human immunoglobulin. For further details, see Jones et al., Nature 321:522-525 (1986); Reichmann et al., Nature 332:323-329 (1988); and Presta, Curr. Op. Struct. Biol. 2:593-596 (1992). See e.g., Ahmed & Cheung, FEBS Letters 588(2):288-297 (2014).

As used herein, the term “hypervariable region” refers to the amino acid residues of an antibody which are responsible for antigen-binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35B (H1), 50-65 (H2) and 95-102 (H3) in the V_{H} (Kabat et al., Sequences of Proteins of Immunological Interest, 5th Ed. Public Health Service, National Institutes of Health, Bethesda, MD. (1991)) and/or those residues from a “hypervariable loop” (e.g., residues 26-32 (L1), 50-52 (L2) and 91-96 (L3) in the V_{L}, and 26-32 (H1), 52A-55 (H2) and 96-101 (H3) in the V_{H} (Chothia and Lesk J. Mol. Biol. 196:901-917 (1987)).

As used herein, the terms “identical” or percent “identity”, when used in the context of two or more nucleic acids or polypeptide sequences, refer to two or more sequences or subsequences that are the same or have a specified percentage of amino acid residues or nucleotides that are the same (i.e., about 60%, 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein)), when compared and aligned for maximum correspondence over a comparison window or designated region as measured using a BLAST or BLAST 2.0 sequence comparison algorithms with default parameters described below, or by manual alignment and visual inspection (e.g., NCBI web site). Such sequences are then said to be “substantially identical.” This term also refers to, or can be applied to, the complement of a test sequence. The term also includes sequences that have deletions and/or additions, as well as those that have substitutions. In some embodiments, identity exists over a region that is at least about 25 amino acids or nucleotides in length, or 50-100 amino acids or nucleotides in length.

As used herein, the term “intact antibody” or “intact immunoglobulin” means an antibody that has at least two heavy (H) chain polypeptides and two light (L) chain polypeptides interconnected by disulfide bonds. Each heavy chain is comprised of a heavy chain variable region (abbreviated herein as HCVR or V_{H}) and a heavy chain constant region. The heavy chain constant region is comprised of three domains, CH₁, CH₂ and CH₃. Each light chain is comprised of a light chain variable region (abbreviated herein as LCVR or V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR₁, CDR₁, FR₂, CDR₂, FR₃, CDR₃, FR₄. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

As used herein, the terms “individual”, “patient”, or “subject” can be an individual organism, a vertebrate, a mammal, or a human. In some embodiments, the individual, patient or subject is a human.

The term “monoclonal antibody” as used herein refers to an antibody obtained from a population of substantially homogeneous antibodies, i.e., the individual antibodies comprising the population are identical except for possible naturally occurring mutations that may be present in minor amounts. For example, a monoclonal antibody can be an antibody that is derived from a single clone, including any eukaryotic, prokaryotic, or phage clone, and not the method by which it is produced. A monoclonal antibody composition displays a single binding specificity and affinity for a particular epitope. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. Monoclonal antibodies can be prepared using a wide variety of techniques known in the art including, e.g., but not limited to, hybridoma, recombinant, and phage display technologies. For example, the monoclonal antibodies to be used in accordance with the present methods may be made by the hybridoma method first described by Kohler et al., Nature 256:495 (1975), or may be made by recombinant DNA methods (See, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington's Pharmaceutical Sciences (20th edition, ed. A. Gennaro, 2000, Lippincott, Williams & Wilkins, Philadelphia, Pa.). Examples of pharmaceutically-acceptable carriers include a liquid or solid filler, diluent, excipient, manufacturing aid (e.g., lubricant, talc magnesium, calcium or zinc stearate, or steric acid), or solvent encapsulating material, useful for introducing the active agent into the body.

As used herein, the term “polyclonal antibody” means a preparation of antibodies derived from at least two (2) different antibody-producing cell lines. The use of this term includes preparations of at least two (2) antibodies that contain antibodies that specifically bind to different epitopes or regions of an antigen.

As used herein, the term “polynucleotide” or “nucleic acid” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, the terms “polypeptide,” “peptide” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art. Such modifications are well described in basic texts and in more detailed monographs, as well as in a voluminous research literature

As used herein, “prevention,” “prevent,” or “preventing” of a disorder or condition refers to one or more compounds that, in a statistical sample, reduces the occurrence of the disorder or condition in the treated sample relative to an untreated control sample, or delays the onset of one or more symptoms of the disorder or condition relative to the untreated control sample.

As used herein, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the material is derived from a cell so modified. Thus, for example, recombinant cells express genes that are not found within the native (non-recombinant) form of the cell or express native genes that are otherwise abnormally expressed, under expressed or not expressed at all.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, “specifically binds” refers to a molecule (e.g., an antibody or antigen binding fragment thereof) which recognizes and binds another molecule (e.g., an antigen), but that does not substantially recognize and bind other molecules. The terms “specific binding,” “specifically binds to,” or is “specific for” a particular molecule (e.g., a polypeptide, or an epitope on a polypeptide), as used herein, can be exhibited, for example, by a molecule having a K_{D} for the molecule to which it binds to of about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. The term “specifically binds” may also refer to binding where a molecule (e.g., an antibody or antigen binding fragment thereof) binds to a particular polypeptide (e.g., a PD-1 polypeptide), or an epitope on a particular polypeptide, without substantially binding to any other polypeptide, or polypeptide epitope.

As used herein, the term “simultaneous” therapeutic use refers to the administration of at least two active ingredients by the same route and at the same time or at substantially the same time.

As used herein, the term “therapeutic agent” is intended to mean a compound that, when present in an effective amount, produces a desired therapeutic effect on a subject in need thereof.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

Amino acid sequence modification(s) of the anti-PD-1 antibodies described herein are contemplated. For example, it may be desirable to improve the binding affinity and/or other biological properties of the antibody. Amino acid sequence variants of an anti-PD-1 antibody are prepared by introducing appropriate nucleotide changes into the antibody nucleic acid, or by peptide synthesis. Such modifications include, for example, deletions from, and/or insertions into and/or substitutions of, residues within the amino acid sequences of the antibody. Any combination of deletion, insertion, and substitution is made to obtain the antibody of interest, as long as the obtained antibody possesses the desired properties. The modification also includes the change of the pattern of glycosylation of the protein. The sites of greatest interest for substitutional mutagenesis include the hypervariable regions, but FR alterations are also contemplated. “Conservative substitutions” are shown in the Table below.

TABLE 1
Amino Acid Substitutions

| Original | Exemplary | Conservative |
|---|---|---|
| Residue | Substitutions | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; | leu |
| | norleucine | |
| Leu (L) | norleucine; ile; val; met; | ile |
| | ala; phe | |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; | leu |
| | norleucine | |

One type of substitutional variant involves substituting one or more hypervariable region residues of a parent antibody. A convenient way for generating such substitutional variants involves affinity maturation using phage display. Specifically, several hypervariable region sites (e.g., 6-7 sites) are mutated to generate all possible amino acid substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and the antigen. Such contact residues and neighboring residues are candidates for substitution according to the techniques elaborated herein. Once such variants are generated, the panel of variants is subjected to screening as described herein and antibodies with similar or superior properties in one or more relevant assays may be selected for further development.

### Immunoglobulin-Related Compositions Comprising Dostarlimab

The present technology describes methods and compositions for the generation and use of dostarlimab immunoglobulin-related compositions (e.g., anti-PD-1 antibodies or antigen binding fragments thereof). The anti-PD-1 immunoglobulin-related compositions of the present disclosure may be useful in the treatment of MMRd rectal cancer (e.g., locally advanced rectal cancer). Anti-PD-1 immunoglobulin-related compositions within the scope of the present technology include, e.g., but are not limited to, monoclonal, chimeric, humanized, bispecific antibodies and diabodies that specifically bind the target polypeptide, a homolog, derivative or a fragment thereof. The present disclosure also provides antigen binding fragments of any of the anti-PD-1 antibodies disclosed herein, wherein the antigen binding fragment is selected from the group consisting of Fab, F(ab)′2, Fab′, scFᵥ, and Fᵥ.

Dostarlimab, sold under the brand name Jemperli, is a monoclonal antibody medication that is indicated for the treatment of adults with mismatch repair deficient (dMMR) recurrent or advanced endometrial cancer, that has progressed on or following prior treatment with a platinum-containing regimen. Dostarlimab is an IgG4 humanized monoclonal antibody that binds with high affinity to PD-1, resulting in inhibition of binding to PD-L1 and PD-L2.

PD-1 is a co-inhibitory receptor that is an important checkpoint protein for regulating T-cell tolerance. When PD-1 is constantly stimulated by PD-1 ligands, which are highly expressed in cancer cells, it allows cancer cells to dodge T-cell mediated immune responses. Therefore, blocking the binding of PD-1 to these ligands can allow T-cells to function normally and prevent tumor cells from bypassing immune surveillance.

Information regarding dostarlimab (or antigen binding fragments thereof) for use in the methods provided herein can be found in U.S. Pat. Nos. 11,155,624 and 9,815,897, the disclosure of which is incorporated herein by reference in its entirety. Sostarlimab and antigen-binding fragments thereof for use in the methods provided herein comprises a heavy chain and a light chain or a heavy chain variable region and a light chain variable region.

Those of ordinary skill in the art would easily be able to identify Chothia-defined, Abm-defined, Kabat-defined or other CDRs.

| | |
|---|---|
| SEQ ID NO: 1 | Dostarlimab V_{L} domain |
| | DIQLTQSPSFLSAYVGDRVTITCKASQDVGTAVAWYQQKPGKAP |
| | KLLIYWASTLHTGVPSRFSGSGSGTEFTLTISSLQPEDFATYYCQH |
| | YSSYPWTFGQGTKLEIK |
| SEQ ID NO: 2 | Dostarlimab Light Chain |
| | DIQLTQSPSFLSAYVGDRVTITCKASQDVGTAVAWYQQKPGKAP |
| | KLLIYWASTLHTGVPSRFSGSGSGTEFTLTISSLQPEDFATYYCQH |
| | YSSYPWTFGQGTKLEIKRTVAAPSVFIFPPSDEQLKSGTASVVCLL |
| | NNFYPREAKVQWKVDNALQSGNSQESVTEQDSKDSTYSLSSTLTL |
| | SKADYEKHKVYACEVTHQGLSSPVTKSFNRGEC |
| SEQ ID NO: 3 | Dostarlimab V_{H} domain |
| | EVQLLESGGGLVQPGGSLRLSCAASGFTFSSYDMSWVRQAPGKG |
| | LEWVSTISGGGSYTYYQDSVKGRFTISRDNSKNTLYLQMNSLRAE |
| | DTAVYYCASPYYAMDYWGQGTTVTVSS |
| SEQ ID NO: 4 | Dostarlimab Heavy Chain |
| | EVQLLESGGGLVQPGGSLRLSCAASGFTFSSYDMSWVRQAPGKG |
| | LEWVSTISGGGSYTYYQDSVKGRFTISRDNSKNTLYLQMNSLRAE |
| | DTAVYYCASPYYAMDYWGQGTTVTVSSASTKGPSVFPLAPCSRS |
| | TSESTAALGCLVKDYFPEPVTVSWNSGALTSGVHTFPAVLQSSGL |
| | YSLSSVVTVPSSSLGTKTYTCNVDHKPSNTKVDKRVESKYGPPCP |
| | PCPAPEFLGGPSVFLFPPKPKDTLMISRTPEVTCVVVDVSQEDPEV |
| | QFNWYVDGVEVHNAKTKPREEQFNSTYRVVSVLTVLHQDWLNG |
| | KEYKCKVSNKGLPSSIEKTISKAKGQPREPQVYTLPPSQEEMTKNQ |
| | VSLTCLVKGFYPSDIAVEWESNGQPENNYKTTPPVLDSDGSFFLYS |
| | RLTVDKSRWQEGNVFSCSVMHEALHNHYTQKSLSLSLGK |
| SEQ ID NO: 5 | Dostarlimab V_{H} CDR1 |
| | SYDMS |
| SEQ ID NO: 6 | Dostarlimab V_{H} CDR2 |
| | TISGGGSYTYYQDSVKG |
| SEQ ID NO: 7 | Dostarlimab V_{H} CDR3 |
| | PYYAMDY |
| SEQ ID NO: 8 | Dostarlimab V_{L} CDR1 |
| | KASQDVGTAVA |
| SEQ ID NO: 9 | Dostarlimab V_{L} CDR2 |
| | WASTLHT |
| SEQ ID NO: 10 | Dostarlimab V_{L} CDR3 |
| | QHYSSYPWT |
| SEQ ID NO: 11 | Dostarlimab Heavy Chain |
| | GAGGTGCAGC TGTTGGAGTC TGGGGGAGGC TTGGTACAGC |
| | CTGGGGGGTCCCTGAGACTC TCCTGTGCAG CCTCTGGATT |
| | CACTTTCAGT AGCTATGACA TGTCTTGGGT CCGCCAGGCT |
| | CCAGGGAAGG GGCTGGAGTG GGTCTCAACC ATTAGTGGTG |
| | GTGGTAGTTA CACCTACTAT CAAGACAGTG TGAAGGGGCG |
| | GTTCACCATC TCCAGAGACA ATTCCAAGAA CACGCTGTAT |
| | CTGCAAATGA ACAGCCTGAG AGCCGAGGAC ACGGCCGTAT |
| | ATTACTGTGC GTCCCCTTAC TATGCTATGG ACTACTGGGG |
| | GCAAGGGACC ACGGTCACCG TCTCCTCAGC ATCCACCAAG |
| | GGCCCATCGG TCTTCCCGCT AGCACCCTGC TCCAGGAGCA |
| | CCTCCGAGAG CACAGCCGCC CTGGGCTGCC TGGTCAAGGA |
| | CTACTTCCCC GAACCAGTGA CGGTGTCGTG GAACTCAGGC |
| | GCCCTGACCA GCGGCGTGCA CACCTTCCCG GCTGTCCTAC |
| | AGTCCTCAGG ACTCTACTCC CTCAGCAGCG TGGTGACCGT |
| | GCCCTCCAGC AGCTTGGGCA CGAAGACCTA CACCTGCAAC |
| | GTAGATCACA AGCCCAGCAA CACCAAGGTG GACAAGAGAG |
| | TTGAGTCCAA ATATGGTCCC CCATGCCCAC CATGCCCAGC |
| | ACCTGAGTTC CTGGGGGGAC CATCAGTCTT CCTGTTCCCC |
| | CCAAAACCCA AGGACACTCT CATGATCTCC CGGACCCCTG |
| | AGGTCACGTG CGTGGTGGTG GACGTGAGCC AGGAAGACCC |
| | CGAGGTCCAG TTCAACTGGT ACGTGGATGG CGTGGAGGTG |
| | CATAATGCCA AGACAAAGCC GCGGGAGGAG CAGTTCAACA |
| | GCACGTACCG TGTGGTCAGC GTCCTCACCG TCCTGCACCA |
| | GGACTGGCTG AACGGCAAGG AGTACAAGTG CAAGGTCTCC |
| | AACAAAGGCC TCCCGTCCTC CATCGAGAAA ACCATCTCCA |
| | AAGCCAAAGG GCAGCCCCGA GAGCCACAGG TGTACACCCT |
| | GCCCCCATCC CAGGAGGAGA TGACCAAGAA CCAGGTCAGC |
| | CTGACCTGCC TGGTCAAAGG CTTCTACCCC AGCGACATCG |
| | CCGTGGAGTG GGAGAGCAAT GGGCAGCCGG AGAACAACTA |
| | CAAGACCACG CCTCCCGTGC TGGACTCCGA CGGCTCCTTC |
| | TTCCTCTACA GCAGGCTAAC CGTGGACAAG AGCAGGTGGC |
| | AGGAGGGGAA TGTCTTCTCA TGCTCCGTGA TGCATGAGGC |
| | TCTGCACAAC CACTACACAC AGAAGAGCCT CTCCCTGTCT |
| | CTGGGTAAA |
| SEQ ID NO: 12 | Dostarlimab Light Chain |
| | GACATCCAGT TGACCCAGTC TCCATCCTTC CTGTCTGCAT |
| | ATGTAGGAGA CAGAGTCACC ATCACTTGCA AGGCCAGTCA |
| | GGATGTGGGT ACTGCTGTAG CCTGGTATCA GCAAAAACCA |
| | GGGAAAGCCC CTAAGCTCCT GATCTATTGG GCATCCACCC |
| | TGCACACTGG GGTCCCATCA AGGTTCAGCG GCAGTGGATC |
| | TGGGACAGAA TTCACTCTCA CAATCAGCAG CCTGCAGCCT |
| | GAAGATTTTG CAACTTATTA CTGTCAGCAT TATAGCAGCT |
| | ATCCGTGGAC GTTTGGCCAG GGGACCAAGC TGGAGATCAA |
| | ACGGACTGTG GCTGCACCAT CTGTCTTCAT CTTCCCGCCA |
| | TCTGATGAGC AATTGAAATC TGGAACTGCC TCTGTTGTGT |
| | GCCTGCTGAA TAACTTCTAT CCCAGAGAGG CCAAAGTACA |
| | GTGGAAGGTG GATAACGCCC TCCAATCGGG TAACTCCCAG |
| | GAGAGTGTCA CAGAGCAGGA CAGCAAGGAC AGCACCTACA |
| | GCCTCAGCAG CACCCTGACG CTGAGCAAAG CAGACTACGA |
| | GAAACACAAA GTCTACGCCT GCGAAGTCAC CCATCAGGGC |
| | CTCAGCTCGC CCGTCACAAA GAGCTTCAAC AGGGGAGAGT |
| | GT |

In one aspect, the present disclosure provides a PD-1 antibody or antigen binding fragment thereof comprising a heavy chain immunoglobulin variable domain (V_{H}) and a light chain immunoglobulin variable domain (V_{L}), wherein the V_{H} comprises a V_{H}-CDR1 sequence of SEQ ID NO: 5, a V_{H}-CDR2 sequence of SEQ ID NO: 6, and a V_{H}-CDR3 sequence of SEQ ID NO: 7 and the V_{L} comprises a V_{L}-CDR1 sequence of SEQ ID NO: 8, a V_{L}—CDR2 sequence of SEQ ID NO: 9, and a V_{L}-CDR3 sequence of SEQ ID NO: 10. In one aspect, the present disclosure provides a PD-1 antibody or antigen binding fragment thereof comprising a heavy chain immunoglobulin variable domain (V_{H}) and a light chain immunoglobulin variable domain (V_{L}), wherein: (a) the V_{H} comprises an amino acid sequence of SEQ ID NO: 3; and (b) the V_{L} comprises an amino acid sequence of SEQ ID NO: 1.

In any of the above embodiments, the antibody further comprises a Fc domain of any isotype, e.g., but are not limited to, IgG (including IgG1, IgG2, IgG3, and IgG4), IgA (including IgA₁ and IgA₂), IgD, IgE, or IgM, and IgY. Non-limiting examples of constant region sequences include:

Human IgD constant region, Uniprot: P01880
(SEQ ID NO: 13)
APTKAPDVFPIISGCRHPKDNSPVVLACLITGYHPTSVTVTWYMGTQSQP
QRTFPEIQRRDSYYMTSSQLSTPLQQWRQGEYKCVVQHTASKSKKEIFRW
PESPKAQASSVPTAQPQAEGSLAKATTAPATTRNTGRGGEEKKKEKEKEE
QEERETKTPECPSHTQPLGVYLLTPAVQDLWLRDKATFTCFVVGSDLKDA
HLTWEVAGKVPTGGVEEGLLERHSNGSQSQHSRLTLPRSLWNAGTSVTCT
LNHPSLPPQRLMALREPAAQAPVKLSLNLLASSDPPEAASWLLCEVSGFS
PPNILLMWLEDQREVNTSGFAPARPPPQPGSTTFWAWSVLRVPAPPSPQP
ATYTCVVSHEDSRTLLNASRSLEVSYVTDHGPMK
Human IgG1 constant region, Uniprot: P01857
(SEQ ID NO: 14)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
Human IgG2 constant region, Uniprot: P01859
(SEQ ID NO: 15)
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSHEDP
EVQFNWYVDGVEVHNAKTKPREEQFNSTFRVVSVLTVVHQDWLNGKEYKC
KVSNKGLPAPIEKTISKTKGQPREPQVYTLPPSREEMTKNQVSLTCLVKG
FYPSDISVEWESNGQPENNYKTTPPMLDSDGSFFLYSKLTVDKSRWQQGN
VFSCSVMHEALHNHYTQKSLSLSPGK
Human IgG3 constant region, Uniprot: P01860
(SEQ ID NO: 16)
ASTKGPSVFPLAPCSRSTSGGTAALGCLVKDYFPEPVTVSWNSGALTSGV
HTFPAVLQSSGLYSLSSVVTVPSSSLGTQTYTCNVNHKPSNTKVDKRVEL
KTPLGDTTHTCPRCPEPKSCDTPPPCPRCPEPKSCDTPPPCPRCPEPKSC
DTPPPCPRCPAPELLGGPSVFLFPPKPKDTLMISRTPEVTCVVVDVSHED
PEVQFKWYVDGVEVHNAKTKPREEQYNSTFRVVSVLTVLHQDWLNGKEYK
CKVSNKALPAPIEKTISKTKGQPREPQVYTLPPSREEMTKNQVSLTCLVK
GFYPSDIAVEWESSGQPENNYNTTPPMLDSDGSFFLYSKLTVDKSRWQQG
NIFSCSVMHEALHNRFTQKSLSLSPGK
Human IgM constant region, Uniprot: P01871
(SEQ ID NO: 17)
GSASAPTLFPLVSCENSPSDTSSVAVGCLAQDFLPDSITLSWKYKNNSDI
SSTRGFPSVLRGGKYAATSQVLLPSKDVMQGTDEHVVCKVQHPNGNKEKN
VPLPVIAELPPKVSVFVPPRDGFFGNPRKSKLICQATGFSPRQIQVSWLR
EGKQVGSGVTTDQVQAEAKESGPTTYKVTSTLTIKESDWLGQSMFTCRVD
HRGLTFQQNASSMCVPDQDTAIRVFAIPPSFASIFLTKSTKLTCLVTDLT
TYDSVTISWTRQNGEAVKTHTNISESHPNATFSAVGEASICEDDWNSGER
FTCTVTHTDLPSPLKQTISRPKGVALHRPDVYLLPPAREQLNLRESATIT
CLVTGFSPADVFVQWMQRGQPLSPEKYVTSAPMPEPQAPGRYFAHSILTV
SEEEWNTGETYTCVAHEALPNRVTERTVDKSTGKPTLYNVSLVMSDTAGT
CY
Human IgG4 constant region, Uniprot: P01861
(SEQ ID NO: 18)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPSCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK
Human IgA1 constant region, Uniprot: P01876
(SEQ ID NO: 19)
ASPTSPKVFPLSLCSTQPDGNVVIACLVQGFFPQEPLSVTWSESGQGVTA
RNFPPSQDASGDLYTTSSQLTLPATQCLAGKSVTCHVKHYTNPSQDVTVP
CPVPSTPPTPSPSTPPTPSPSCCHPRLSLHRPALEDLLLGSEANLTCTLT
GLRDASGVTFTWTPSSGKSAVQGPPERDLCGCYSVSSVLPGCAEPWNHGK
TFTCTAAYPESKTPLTATLSKSGNTFRPEVHLLPPPSEELALNELVTLTC
LARGFSPKDVLVRWLQGSQELPREKYLTWASRQEPSQGTTTFAVTSILRV
AAEDWKKGDTFSCMVGHEALPLAFTQKTIDRLAGKPTHVNVSVVMAEVDG
TCY
Human IgA2 constant region, Uniprot: P01877
(SEQ ID NO: 20)
ASPTSPKVFPLSLDSTPQDGNVVVACLVQGFFPQEPLSVTWSESGQNVTA
RNFPPSQDASGDLYTTSSQLTLPATQCPDGKSVTCHVKHYTNPSQDVTVP
CPVPPPPPCCHPRLSLHRPALEDLLLGSEANLTCTLTGLRDASGATFTWT
PSSGKSAVQGPPERDLCGCYSVSSVLPGCAQPWNHGETFTCTAAHPELKT
PLTANITKSGNTFRPEVHLLPPPSEELALNELVTLTCLARGFSPKDVLVR
WLQGSQELPREKYLTWASRQEPSQGTTTFAVTSILRVAAEDWKKGDTFSC
MVGHEALPLAFTQKTIDRMAGKPTHVNVSVVMAEVDGTCY
Human Ig kappa constant region, Uniprot: P01834
(SEQ ID NO: 21)
TVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKS
FNRGEC

In some embodiments, the immunoglobulin-related compositions of the present technology comprise a heavy chain constant region that is at least 80%, at least 85%, at least 90%, at least 95%, at least 99%, or is 100% identical to SEQ ID NOS: 13-20. Additionally or alternatively, in some embodiments, the immunoglobulin-related compositions of the present technology comprise a light chain constant region that is at least 80%, at least 85%, at least 90%, at least 95%, at least 99%, or is 100% identical to SEQ ID NO: 21.

In some embodiments, the anti-PD-1 immunoglobulin-related compositions of the present technology bind to an epitope within the extracellular region of human PD-1. In certain embodiments, the epitope is a conformational epitope or non-conformational epitope.

In another aspect, the present disclosure provides an isolated immunoglobulin-related composition (e.g., an antibody or antigen binding fragment thereof) comprising a heavy chain (HC) amino acid sequence comprising a heavy chain (HC) amino acid sequence comprising SEQ ID NO: 4, or a variant thereof having one or more conservative amino acid substitutions. Additionally or alternatively, in some embodiments, the immunoglobulin-related compositions of the present technology comprise a light chain (LC) amino acid sequence comprising SEQ ID NO: 2, or a variant thereof having one or more conservative amino acid substitutions. In some embodiments, the immunoglobulin-related compositions of the present technology comprise a HC amino acid sequence comprising SEQ ID NO: 4 and a LC amino acid sequence comprising SEQ ID NO: 2.

In some embodiments, the HC and LC immunoglobulin variable domain sequences are components of the same polypeptide chain. In other embodiments, the HC and LC immunoglobulin variable domain sequences are components of different polypeptide chains. In certain embodiments, the antibody is a full-length antibody.

In some embodiments, the immunoglobulin-related compositions of the present technology bind specifically to at least one PD-1 polypeptide. In some embodiments, the immunoglobulin-related compositions of the present technology bind at least one PD-1 polypeptide with a dissociation constant (K_{D}) of about 10⁻³M, 10⁻⁴ M, 10⁻⁵ M, 10⁻⁶ M, 10⁻⁷ M, 10⁻⁸M, 10⁻⁹M, 10⁻¹⁰ M, 10⁻¹¹ M, or 10⁻¹² M. In certain embodiments, the immunoglobulin-related compositions are monoclonal antibodies, chimeric antibodies, humanized antibodies, bispecific antibodies, or multi-specific antibodies. In some embodiments, the antibodies comprise a human antibody framework region.

In certain embodiments, the immunoglobulin-related composition includes one or more of the following characteristics: (a) a light chain immunoglobulin variable domain sequence that is at least 80%, at least 85%, at least 90%, at least 95%, or at least 99% identical to the light chain immunoglobulin variable domain sequence of SEQ ID NO: 1; and/or (b) a heavy chain immunoglobulin variable domain sequence that is at least 80%, at least 85%, at least 90%, at least 95%, or at least 99% identical to the heavy chain immunoglobulin variable domain sequence of SEQ ID NO: 3. In another aspect, one or more amino acid residues in the immunoglobulin-related compositions provided herein are substituted with another amino acid. The substitution may be a “conservative substitution” as defined herein.

In another aspect, the present disclosure provides an antibody comprising (a) a LC sequence that is at least 80%, at least 85%, at least 90%, at least 95%, or at least 99% identical to the LC sequence present in SEQ ID NO: 2; and/or (b) a HC sequence that is at least 80%, at least 85%, at least 90%, at least 95%, or at least 99% identical to the HC sequence present in SEQ ID NO: 4.

In certain embodiments, the immunoglobulin-related compositions contain an IgG1 constant region comprising one or more amino acid substitutions selected from the group consisting of N297A and K322A. Additionally or alternatively, in some embodiments, the immunoglobulin-related compositions contain an IgG4 constant region comprising a S228P mutation.

In some aspects, the anti-PD-1 immunoglobulin-related compositions described herein contain structural modifications to facilitate rapid binding and cell uptake and/or slow release. In some aspects, the anti-PD-1 immunoglobulin-related composition of the present technology (e.g., an antibody) may contain a deletion in the CH2 constant heavy chain region to facilitate rapid binding and cell uptake and/or slow release. In some aspects, a Fab fragment is used to facilitate rapid binding and cell uptake and/or slow release. In some aspects, a F(ab)′₂ fragment is used to facilitate rapid binding and cell uptake and/or slow release.

In one aspect, the present technology provides a nucleic acid sequence encoding any of the immunoglobulin-related compositions described herein. Also disclosed herein are recombinant nucleic acid sequences selected from among SEQ ID NO: 11 and SEQ ID NO: 12. In another aspect, the present technology provides a host cell expressing any nucleic acid sequence encoding any of the immunoglobulin-related compositions described herein.

The immunoglobulin-related compositions of the present technology (e.g., an anti-PD-1 antibody) can be monospecific, bispecific, trispecific or of greater multi-specificity. Multi-specific antibodies can be specific for different epitopes of PD-1 polypeptides as well as for heterologous compositions, such as a heterologous polypeptide or solid support material. See, e.g., WO 93/17715; WO 92/08802; WO 91/00360; WO 92/05793; Tutt et al., J. Immunol. 147: 60-69 (1991); U.S. Pat. Nos. 5,573,920, 4,474,893, 5,601,819, 4,714,681, 4,925,648; 6,106,835; Kostelny et al., J. Immunol. 148: 1547-1553 (1992). In some embodiments, the immunoglobulin-related compositions are chimeric. In certain embodiments, the immunoglobulin-related compositions are humanized.

The immunoglobulin-related compositions of the present technology can further be recombinantly fused to a heterologous polypeptide at the N- or C-terminus or chemically conjugated (including covalently and non-covalently conjugations) to polypeptides or other compositions. For example, the immunoglobulin-related compositions of the present technology can be recombinantly fused or conjugated to molecules useful as labels in detection assays and effector molecules such as heterologous polypeptides, drugs, or toxins. See, e.g., WO 92/08495; WO 91/14438; WO 89/12624; U.S. Pat. No. 5,314,995; and EP 0 396 387.

In any of the above embodiments of the immunoglobulin-related compositions of the present technology, the antibody or antigen binding fragment may be optionally conjugated to an agent selected from the group consisting of isotopes, dyes, chromagens, contrast agents, drugs, toxins, cytokines, enzymes, enzyme inhibitors, hormones, hormone antagonists, growth factors, radionuclides, metals, liposomes, nanoparticles, RNA, DNA or any combination thereof. For a chemical bond or physical bond, a functional group on the immunoglobulin-related composition typically associates with a functional group on the agent. Alternatively, a functional group on the agent associates with a functional group on the immunoglobulin-related composition.

The functional groups on the agent and immunoglobulin-related composition can associate directly. For example, a functional group (e.g., a sulfhydryl group) on an agent can associate with a functional group (e.g., sulfhydryl group) on an immunoglobulin-related composition to form a disulfide. Alternatively, the functional groups can associate through a cross-linking agent (i.e., linker). Some examples of cross-linking agents are described below. The cross-linker can be attached to either the agent or the immunoglobulin-related composition. The number of agents or immunoglobulin-related compositions in a conjugate is also limited by the number of functional groups present on the other. For example, the maximum number of agents associated with a conjugate depends on the number of functional groups present on the immunoglobulin-related composition. Alternatively, the maximum number of immunoglobulin-related compositions associated with an agent depends on the number of functional groups present on the agent.

In yet another embodiment, the conjugate comprises one immunoglobulin-related composition associated to one agent. In one embodiment, a conjugate comprises at least one agent chemically bonded (e.g., conjugated) to at least one immunoglobulin-related composition. The agent can be chemically bonded to an immunoglobulin-related composition by any method known to those in the art. For example, a functional group on the agent may be directly attached to a functional group on the immunoglobulin-related composition. Some examples of suitable functional groups include, for example, amino, carboxyl, sulfhydryl, maleimide, isocyanate, isothiocyanate and hydroxyl.

The agent may also be chemically bonded to the immunoglobulin-related composition by means of cross-linking agents, such as dialdehydes, carbodiimides, dimaleimides, and the like. Cross-linking agents can, for example, be obtained from Pierce Biotechnology, Inc., Rockford, Ill. The Pierce Biotechnology, Inc. web-site can provide assistance. Additional cross-linking agents include the platinum cross-linking agents described in U.S. Pat. Nos. 5,580,990; 5,985,566; and 6,133,038 of Kreatech Biotechnology, B.V., Amsterdam, The Netherlands.

Alternatively, the functional group on the agent and immunoglobulin-related composition can be the same. Homobifunctional cross-linkers are typically used to cross-link identical functional groups. Examples of homobifunctional cross-linkers include EGS (i.e., ethylene glycol bis[succinimidylsuccinate]), DSS (i.e., disuccinimidyl suberate), DMA (i.e., dimethyl adipimidate·2HCl), DTSSP (i.e., 3,3′-dithiobis[sulfosuccinimidylpropionate])), DPDPB (i.e., 1,4-di-[3′-(2′-pyridyldithio)-propionamido]butane), and BMH (i.e., bis-maleimidohexane). Such homobifunctional cross-linkers are also available from Pierce Biotechnology, Inc.

In other instances, it may be beneficial to cleave the agent from the immunoglobulin-related composition. The web-site of Pierce Biotechnology, Inc. described above can also provide assistance to one skilled in the art in choosing suitable cross-linkers which can be cleaved by, for example, enzymes in the cell. Thus the agent can be separated from the immunoglobulin-related composition. Examples of cleavable linkers include SMPT (i.e., 4-succinimidyloxycarbonyl-methyl-a-[2-pyridyldithio]toluene), Sulfo-LC-SPDP (i.e., sulfosuccinimidyl 6-(3-[2-pyridyldithio]-propionamido)hexanoate), LC-SPDP (i.e., succinimidyl 6-(3-[2-pyridyldithio]-propionamido)hexanoate), Sulfo-LC-SPDP (i.e., sulfosuccinimidyl 6-(3-[2-pyridyldithio]-propionamido)hexanoate), SPDP (i.e., N-succinimidyl 3-[2-pyridyldithio]-propionamidohexanoate), and AEDP (i.e., 3-[(2-aminoethyl)dithio]propionic acid HCl).

In another embodiment, a conjugate comprises at least one agent physically bonded with at least one immunoglobulin-related composition. Any method known to those in the art can be employed to physically bond the agents with the immunoglobulin-related compositions. For example, the immunoglobulin-related compositions and agents can be mixed together by any method known to those in the art. The order of mixing is not important. For instance, agents can be physically mixed with immunoglobulin-related compositions by any method known to those in the art. For example, the immunoglobulin-related compositions and agents can be placed in a container and agitated, by for example, shaking the container, to mix the immunoglobulin-related compositions and agents.

The immunoglobulin-related compositions can be modified by any method known to those in the art. For instance, the immunoglobulin-related composition may be modified by means of cross-linking agents or functional groups, as described above.

### Methods of Preparing Anti-PD1 Antibodies of the Present Technology

General Overview. Initially, a target polypeptide is chosen to which an antibody of the present technology can be raised. For example, an antibody may be raised against the full-length PD-1 protein, or to a portion of the extracellular domain of the PD-1 protein. Techniques for generating antibodies directed to such target polypeptides are well known to those skilled in the art. Examples of such techniques include, for example, but are not limited to, those involving display libraries, xeno or human mice, hybridomas, and the like. Target polypeptides within the scope of the present technology include any polypeptide derived from PD-1 protein containing the extracellular domain which is capable of eliciting an immune response.

It should be understood that recombinantly engineered antibodies and antibody fragments, e.g., antibody-related polypeptides, which are directed to PD-1 protein and fragments thereof are suitable for use in accordance with the present disclosure.

Anti-PD1 antibodies that can be subjected to the techniques set forth herein include monoclonal and polyclonal antibodies, and antibody fragments such as Fab, Fab′, F(ab′)₂, Fd, scFv, diabodies, antibody light chains, antibody heavy chains and/or antibody fragments. Methods useful for the high yield production of antibody Fv-containing polypeptides, e.g., Fab′ and F(ab′)₂ antibody fragments have been described. See U.S. Pat. No. 5,648,237.

Generally, an antibody is obtained from an originating species. More particularly, the nucleic acid or amino acid sequence of the variable portion of the light chain, heavy chain or both, of an originating species antibody having specificity for a target polypeptide antigen is obtained. An originating species is any species which was useful to generate the antibody of the present technology or library of antibodies, e.g., rat, mouse, rabbit, chicken, monkey, human, and the like.

Phage or phagemid display technologies are useful techniques to derive the antibodies of the present technology. Techniques for generating and cloning monoclonal antibodies are well known to those skilled in the art. Expression of sequences encoding antibodies of the present technology, can be carried out in E. coli.

Due to the degeneracy of nucleic acid coding sequences, other sequences which encode substantially the same amino acid sequences as those of the naturally occurring proteins may be used in the practice of the present technology These include, but are not limited to, nucleic acid sequences including all or portions of the nucleic acid sequences encoding the above polypeptides, which are altered by the substitution of different codons that encode a functionally equivalent amino acid residue within the sequence, thus producing a silent change. It is appreciated that the nucleotide sequence of an immunoglobulin according to the present technology tolerates sequence homology variations of up to 25% as calculated by standard methods (“Current Methods in Sequence Comparison and Analysis,” Macromolecule Sequencing and Synthesis, Selected Methods and Applications, pp. 127-149, 1998, Alan R. Liss, Inc.) so long as such a variant forms an operative antibody which recognizes PD-1 proteins. For example, one or more amino acid residues within a polypeptide sequence can be substituted by another amino acid of a similar polarity which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Also included within the scope of the present technology are proteins or fragments or derivatives thereof which are differentially modified during or after translation, e.g., by glycosylation, proteolytic cleavage, linkage to an antibody molecule or other cellular ligands, etc. Additionally, an immunoglobulin encoding nucleic acid sequence can be mutated in vitro or in vivo to create and/or destroy translation, initiation, and/or termination sequences or to create variations in coding regions and/or form new restriction endonuclease sites or destroy pre-existing ones, to facilitate further in vitro modification. Any technique for mutagenesis known in the art can be used, including but not limited to in vitro site directed mutagenesis, J. Biol. Chem. 253:6551, use of Tab linkers (Pharmacia), and the like.

Monoclonal Antibody. In one embodiment of the present technology, the antibody is an anti-PD-1 monoclonal antibody. For example, in some embodiments, the anti-PD-1 monoclonal antibody may be a human or a mouse anti-PD-1 monoclonal antibody. For preparation of monoclonal antibodies directed towards the PD-1 protein, or derivatives, fragments, analogs or homologs thereof, any technique that provides for the production of antibody molecules by continuous cell line culture can be utilized. Such techniques include, but are not limited to, the hybridoma technique (See, e.g., Kohler & Milstein, 1975. Nature 256: 495-497); the trioma technique; the human B-cell hybridoma technique (See, e.g., Kozbor, et al., 1983. Immunol. Today 4: 72) and the EBV hybridoma technique to produce human monoclonal antibodies (See, e.g., Cole, et al., 1985. In: MONOCLONAL ANTIBODIES AND CANCER THERAPY, Alan R. Liss, Inc., pp. 77-96). Human monoclonal antibodies can be utilized in the practice of the present technology and can be produced by using human hybridomas (See, e.g., Cote, et al., 1983. Proc. Natl. Acad. Sci. USA 80: 2026-2030) or by transforming human B-cells with Epstein Barr Virus in vitro (See, e.g., Cole, et al., 1985. In: MONOCLONAL ANTIBODIES AND CANCER THERAPY, Alan R. Liss, Inc., pp. 77-96). For example, a population of nucleic acids that encode regions of antibodies can be isolated. PCR utilizing primers derived from sequences encoding conserved regions of antibodies is used to amplify sequences encoding portions of antibodies from the population and then DNAs encoding antibodies or fragments thereof, such as variable domains, are reconstructed from the amplified sequences. Such amplified sequences also can be fused to DNAs encoding other proteins—e.g., a bacteriophage coat, or a bacterial cell surface protein—for expression and display of the fusion polypeptides on phage or bacteria. Amplified sequences can then be expressed and further selected or isolated based, e.g., on the affinity of the expressed antibody or fragment thereof for an antigen or epitope present on the PD-1 protein. Alternatively, hybridomas expressing anti-PD-1 monoclonal antibodies can be prepared by immunizing a subject and then isolating hybridomas from the subject's spleen using routine methods. See, e.g., Milstein et al., (Galfre and Milstein, Methods Enzymol (1981) 73: 3-46). Screening the hybridomas using standard methods will produce monoclonal antibodies of varying specificity (i.e., for different epitopes) and affinity. A selected monoclonal antibody with the desired properties, e.g., PD-1 binding, can be used as expressed by the hybridoma, it can be bound to a molecule such as polyethylene glycol (PEG) to alter its properties, or a cDNA encoding it can be isolated, sequenced and manipulated in various ways. Synthetic dendromeric trees can be added to reactive amino acid side chains, e.g., lysine, to enhance the immunogenic properties of PD-1 protein. Also, CPG-dinucleotide techniques can be used to enhance the immunogenic properties of the PD-1 protein. Other manipulations include substituting or deleting particular amino acyl residues that contribute to instability of the antibody during storage or after administration to a subject, and affinity maturation techniques to improve affinity of the antibody of the PD-1 protein.

Hybridoma Technique. In some embodiments, the antibody of the present technology is an anti-PD-1 monoclonal antibody produced by a hybridoma which includes a B cell obtained from a transgenic non-human animal, e.g., a transgenic mouse, having a genome comprising a human heavy chain transgene and a light chain transgene fused to an immortalized cell. Hybridoma techniques include those known in the art and taught in Harlow et al., Antibodies: A Laboratory Manual Cold Spring Harbor Laboratory, Cold Spring Harbor, NY, 349 (1988); Hammerling et al., Monoclonal Antibodies And T-Cell Hybridomas, 563-681 (1981). Other methods for producing hybridomas and monoclonal antibodies are well known to those of skill in the art.

Phage Display Technique. As noted above, the antibodies of the present technology can be produced through the application of recombinant DNA and phage display technology. For example, anti-PD-1 antibodies, can be prepared using various phage display methods known in the art. In phage display methods, functional antibody domains are displayed on the surface of a phage particle which carries polynucleotide sequences encoding them. Phages with a desired binding property are selected from a repertoire or combinatorial antibody library (e.g., human or murine) by selecting directly with an antigen, typically an antigen bound or captured to a solid surface or bead. Phages used in these methods are typically filamentous phage including fd and M13 with Fab, Fv or disulfide stabilized Fv antibody domains that are recombinantly fused to either the phage gene III or gene VIII protein. In addition, methods can be adapted for the construction of Fab expression libraries (See, e.g., Huse, et al., Science 246: 1275-1281, 1989) to allow rapid and effective identification of monoclonal Fab fragments with the desired specificity for an PD-1 polypeptide, e.g., a polypeptide or derivatives, fragments, analogs or homologs thereof. Other examples of phage display methods that can be used to make the antibodies of the present technology include those disclosed in Huston et al., Proc. Natl. Acad. Sci U.S.A., 85: 5879-5883, 1988; Chaudhary et al., Proc. Natl. Acad. Sci U.S.A., 87: 1066-1070, 1990; Brinkman et al., J. Immunol. Methods 182: 41-50, 1995; Ames et al., J. Immunol. Methods 184: 177-186, 1995; Kettleborough et al., Eur. J. Immunol. 24: 952-958, 1994; Persic et al., Gene 187: 9-18, 1997; Burton et al., Advances in Immunology 57: 191-280, 1994; PCT/GB91/01134; WO 90/02809; WO 91/10737; WO 92/01047; WO 92/18619; WO 93/11236; WO 95/15982; WO 95/20401; WO 96/06213; WO 92/01047 (Medical Research Council et al.); WO 97/08320 (Morphosys); WO 92/01047 (CAT/MRC); WO 91/17271 (Affymax); and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727 and 5,733,743. Methods useful for displaying polypeptides on the surface of bacteriophage particles by attaching the polypeptides via disulfide bonds have been described by Lohning, U.S. Pat. No. 6,753,136. As described in the above references, after phage selection, the antibody coding regions from the phage can be isolated and used to generate whole antibodies, including human antibodies, or any other desired antigen binding fragment, and expressed in any desired host including mammalian cells, insect cells, plant cells, yeast, and bacteria. For example, techniques to recombinantly produce Fab, Fab′ and F(ab′)₂ fragments can also be employed using methods known in the art such as those disclosed in WO 92/22324; Mullinax et al., BioTechniques 12: 864-869, 1992; and Sawai et al., AJRI 34: 26-34, 1995; and Better et al., Science 240: 1041-1043, 1988.

Generally, hybrid antibodies or hybrid antibody fragments that are cloned into a display vector can be selected against the appropriate antigen in order to identify variants that maintain good binding activity, because the antibody or antibody fragment will be present on the surface of the phage or phagemid particle. See, e.g., Barbas III et al., Phage Display, A Laboratory Manual (Cold Spring Harbor Laboratory Press, Cold Spring Harbor, N.Y., 2001). However, other vector formats could be used for this process, such as cloning the antibody fragment library into a lytic phage vector (modified T7 or Lambda Zap systems) for selection and/or screening.

Expression of Recombinant Anti-PD1 Antibodies. As noted above, the antibodies of the present technology can be produced through the application of recombinant DNA technology. Recombinant polynucleotide constructs encoding an anti-PD-1 antibody of the present technology typically include an expression control sequence operably-linked to the coding sequences of anti-PD-1 antibody chains, including naturally-associated or heterologous promoter regions. As such, another aspect of the technology includes vectors containing one or more nucleic acid sequences encoding an anti-PD-1 antibody of the present technology. For recombinant expression of one or more of the polypeptides of the present technology, the nucleic acid containing all or a portion of the nucleotide sequence encoding the anti-PD-1 antibody is inserted into an appropriate cloning vector, or an expression vector (i.e., a vector that contains the necessary elements for the transcription and translation of the inserted polypeptide coding sequence) by recombinant DNA techniques well known in the art and as detailed below. Methods for producing diverse populations of vectors have been described by Lerner et al., U.S. Pat. Nos. 6,291,160 and 6,680,192.

In general, expression vectors useful in recombinant DNA techniques are often in the form of plasmids. In the present disclosure, “plasmid” and “vector” can be used interchangeably as the plasmid is the most commonly used form of vector. However, the present technology is intended to include such other forms of expression vectors that are not technically plasmids, such as viral vectors (e.g., replication defective retroviruses, adenoviruses and adeno-associated viruses), which serve equivalent functions. Such viral vectors permit infection of a subject and expression of a construct in that subject. In some embodiments, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences encoding the anti-PD-1 antibody, and the collection and purification of the anti-PD-1 antibody, e.g., cross-reacting anti-PD-1 antibodies. See generally, U.S. 2002/0199213. These expression vectors are typically replicable in the host organisms either as episomes or as an integral part of the host chromosomal DNA. Commonly, expression vectors contain selection markers, e.g., ampicillin-resistance or hygromycin-resistance, to permit detection of those cells transformed with the desired DNA sequences. Vectors can also encode signal peptide, e.g., pectate lyase, useful to direct the secretion of extracellular antibody fragments. See U.S. Pat. No. 5,576,195.

The recombinant expression vectors of the present technology comprise a nucleic acid encoding a protein with PD-1 binding properties in a form suitable for expression of the nucleic acid in a host cell, which means that the recombinant expression vectors include one or more regulatory sequences, selected on the basis of the host cells to be used for expression that is operably-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, e.g., in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of polypeptide desired, etc. Typical regulatory sequences useful as promoters of recombinant polypeptide expression (e.g., anti-PD-1 antibody), include, e.g., but are not limited to, promoters of 3-phosphoglycerate kinase and other glycolytic enzymes. Inducible yeast promoters include, among others, promoters from alcohol dehydrogenase, isocytochrome C, and enzymes responsible for maltose and galactose utilization. In one embodiment, a polynucleotide encoding an anti-PD-1 antibody of the present technology is operably-linked to an ara B promoter and expressible in a host cell. See U.S. Pat. No. 5,028,530. The expression vectors of the present technology can be introduced into host cells to thereby produce polypeptides or peptides, including fusion polypeptides, encoded by nucleic acids as described herein (e.g., anti-PD-1 antibody, etc.).

Another aspect of the present technology pertains to anti-PD-1 antibody-expressing host cells, which contain a nucleic acid encoding one or more anti-PD-1 antibodies. The recombinant expression vectors of the present technology can be designed for expression of an anti-PD-1 antibody in prokaryotic or eukaryotic cells. For example, an anti-PD-1 antibody can be expressed in bacterial cells such as Escherichia coli, insect cells (using baculovirus expression vectors), fungal cells, e.g., yeast, yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, e.g., using T7 promoter regulatory sequences and T7 polymerase. Methods useful for the preparation and screening of polypeptides having a predetermined property, e.g., anti-PD-1 antibody, via expression of stochastically generated polynucleotide sequences has been previously described. See U.S. Pat. Nos. 5,763,192; 5,723,323; 5,814,476; 5,817,483; 5,824,514; 5,976,862; 6,492,107; 6,569,641.

Expression of polypeptides in prokaryotes is most often carried out in E. coli with vectors containing constitutive or inducible promoters directing the expression of either fusion or non-fusion polypeptides. Fusion vectors add a number of amino acids to a polypeptide encoded therein, usually to the amino terminus of the recombinant polypeptide. Such fusion vectors typically serve three purposes: (i) to increase expression of recombinant polypeptide; (ii) to increase the solubility of the recombinant polypeptide; and (iii) to aid in the purification of the recombinant polypeptide by acting as a ligand in affinity purification. Often, in fusion expression vectors, a proteolytic cleavage site is introduced at the junction of the fusion moiety and the recombinant polypeptide to enable separation of the recombinant polypeptide from the fusion moiety subsequent to purification of the fusion polypeptide. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding polypeptide, or polypeptide A, respectively, to the target recombinant polypeptide.

Examples of suitable inducible non-fusion E. coli expression vectors include pTrc (Amrann et al., (1988) Gene 69: 301-315) and pET 11d (Studier et al., GENE EXPRESSION TECHNOLOGY: METHODS IN ENZYMOLOGY 185, Academic Press, San Diego, Calif (1990) 60-89). Methods for targeted assembly of distinct active peptide or protein domains to yield multifunctional polypeptides via polypeptide fusion has been described by Pack et al., U.S. Pat. Nos. 6,294,353; 6,692,935. One strategy to maximize recombinant polypeptide expression, e.g., an anti-PD-1 antibody, in E. coli is to express the polypeptide in host bacteria with an impaired capacity to proteolytically cleave the recombinant polypeptide. See, e.g., Gottesman, GENE EXPRESSION TECHNOLOGY: METHODS IN ENZYMOLOGY 185, Academic Press, San Diego, Calif (1990) 119-128. Another strategy is to alter the nucleic acid sequence of the nucleic acid to be inserted into an expression vector so that the individual codons for each amino acid are those preferentially utilized in the expression host, e.g., E. coli (See, e.g., Wada, et al., 1992. Nucl. Acids Res. 20: 2111-2118). Such alteration of nucleic acid sequences of the present technology can be carried out by standard DNA synthesis techniques.

In another embodiment, the anti-PD-1 antibody expression vector is a yeast expression vector. Examples of vectors for expression in yeast Saccharomyces cerevisiae include pYepSecl (Baldari, et al., 1987. EMBO J 6: 229-234), pMFa (Kurjan and Herskowitz, Cell 30: 933-943, 1982), pJRY88 (Schultz et al., Gene 54: 113-123, 1987), pYES2 (Invitrogen Corporation, San Diego, Calif.), and picZ (Invitrogen Corp, San Diego, Calif.). Alternatively, an anti-PD-1 antibody can be expressed in insect cells using baculovirus expression vectors. Baculovirus vectors available for expression of polypeptides, e.g., anti-PD-1 antibody, in cultured insect cells (e.g., SF9 cells) include the pAc series (Smith, et al., Mol. Cell. Biol. 3: 2156-2165, 1983) and the pVL series (Lucklow and Summers, 1989. Virology 170: 31-39).

In yet another embodiment, a nucleic acid encoding an anti-PD-1 antibody of the present technology is expressed in mammalian cells using a mammalian expression vector. Examples of mammalian expression vectors include, e.g., but are not limited to, pCDM8 (Seed, Nature 329: 840, 1987) and pMT2PC (Kaufman, et al., EMBO J. 6: 187-195, 1987). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells that are useful for expression of the anti-PD-1 antibody of the present technology, see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid in a particular cell type (e.g., tissue-specific regulatory elements). Tissue-specific regulatory elements are known in the art. Non-limiting examples of suitable tissue-specific promoters include the albumin promoter (liver-specific; Pinkert, et al., Genes Dev. 1: 268-277, 1987), lymphoid-specific promoters (Calame and Eaton, Adv. Immunol. 43: 235-275, 1988), promoters of T cell receptors (Winoto and Baltimore, EMBO J. 8: 729-733, 1989) and immunoglobulins (Banerji, et al., 1983. Cell 33: 729-740; Queen and Baltimore, Cell 33: 741-748, 1983.), neuron-specific promoters (e.g., the neurofilament promoter; Byrne and Ruddle, Proc. Natl. Acad. Sci. USA 86: 5473-5477, 1989), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, Science 249: 374-379, 1990) and the α-fetoprotein promoter (Campes and Tilghman, Genes Dev. 3: 537-546, 1989).

Another aspect of the present methods pertains to host cells into which a recombinant expression vector of the present technology has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, an anti-PD-1 antibody can be expressed in bacterial cells such as E. coli, insect cells, yeast or mammalian cells. Mammalian cells are a suitable host for expressing nucleotide segments encoding immunoglobulins or fragments thereof. See Winnacker, From Genes To Clones, (VCH Publishers, N Y, 1987). A number of suitable host cell lines capable of secreting intact heterologous proteins have been developed in the art, and include Chinese hamster ovary (CHO) cell lines, various COS cell lines, HeLa cells, L cells and myeloma cell lines. In some embodiments, the cells are non-human. Expression vectors for these cells can include expression control sequences, such as an origin of replication, a promoter, an enhancer, and necessary processing information sites, such as ribosome binding sites, RNA splice sites, polyadenylation sites, and transcriptional terminator sequences. Queen et al., Immunol. Rev. 89: 49, 1986. Illustrative expression control sequences are promoters derived from endogenous genes, cytomegalovirus, SV40, adenovirus, bovine papillomavirus, and the like. Co et al., J Immunol. 148: 1149, 1992. Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, electroporation, biolistics or viral-based transfection. Other methods used to transform mammalian cells include the use of polybrene, protoplast fusion, liposomes, electroporation, and microinjection (See generally, Sambrook et al., Molecular Cloning). Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals. The vectors containing the DNA segments of interest can be transferred into the host cell by well-known methods, depending on the type of cellular host.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding the anti-PD-1 antibody or can be introduced on a separate vector. Cells stably transfected with the introduced nucleic acid can be identified by drug selection (e.g., cells that have incorporated the selectable marker gene will survive, while the other cells die).

A host cell that includes an anti-PD-1 antibody of the present technology, such as a prokaryotic or eukaryotic host cell in culture, can be used to produce (i.e., express) recombinant anti-PD-1 antibody. In one embodiment, the method comprises culturing the host cell (into which a recombinant expression vector encoding the anti-PD-1 antibody has been introduced) in a suitable medium such that the anti-PD-1 antibody is produced. In another embodiment, the method further comprises the step of isolating the anti-PD-1 antibody from the medium or the host cell. Once expressed, collections of the anti-PD-1 antibody, e.g., the anti-PD-1 antibodies or the anti-PD-1 antibody-related polypeptides are purified from culture media and host cells. The anti-PD-1 antibody can be purified according to standard procedures of the art, including HPLC purification, column chromatography, gel electrophoresis and the like. In one embodiment, the anti-PD-1 antibody is produced in a host organism by the method of Boss et al., U.S. Pat. No. 4,816,397. Usually, anti-PD-1 antibody chains are expressed with signal sequences and are thus released to the culture media. However, if the anti-PD-1 antibody chains are not naturally secreted by host cells, the anti-PD-1 antibody chains can be released by treatment with mild detergent. Purification of recombinant polypeptides is well known in the art and includes ammonium sulfate precipitation, affinity chromatography purification technique, column chromatography, ion exchange purification technique, gel electrophoresis and the like (See generally Scopes, Protein Purification (Springer-Verlag, N.Y., 1982).

Polynucleotides encoding anti-PD-1 antibodies, e.g., the anti-PD-1 antibody coding sequences, can be incorporated in transgenes for introduction into the genome of a transgenic animal and subsequent expression in the milk of the transgenic animal. See, e.g., U.S. Pat. Nos. 5,741,957, 5,304,489, and 5,849,992. Suitable transgenes include coding sequences for light and/or heavy chains in operable linkage with a promoter and enhancer from a mammary gland specific gene, such as casein or β-lactoglobulin. For production of transgenic animals, transgenes can be microinjected into fertilized oocytes, or can be incorporated into the genome of embryonic stem cells, and the nuclei of such cells transferred into enucleated oocytes.

Single-Chain Antibodies. In one embodiment, the anti-PD-1 antibody of the present technology is a single-chain anti-PD-1 antibody. According to the present technology, techniques can be adapted for the production of single-chain antibodies specific to an PD-1 protein (See, e.g., U.S. Pat. No. 4,946,778). Examples of techniques which can be used to produce single-chain Fvs and antibodies of the present technology include those described in U.S. Pat. Nos. 4,946,778 and 5,258,498; Huston et al., Methods in Enzymology, 203: 46-88, 1991; Shu, L. et al., Proc. Natl. Acad. Sci. USA, 90: 7995-7999, 1993; and Skerra et al., Science 240: 1038-1040, 1988.

Chimeric and Humanized Antibodies. In one embodiment, the anti-PD-1 antibody of the present technology is a chimeric anti-PD-1 antibody. In one embodiment, the anti-PD-1 antibody of the present technology is a humanized anti-PD-1 antibody. In one embodiment of the present technology, the donor and acceptor antibodies are monoclonal antibodies from different species. For example, the acceptor antibody is a human antibody (to minimize its antigenicity in a human), in which case the resulting CDR-grafted antibody is termed a “humanized” antibody.

Recombinant anti-PD-1 antibodies, such as chimeric and humanized monoclonal antibodies, comprising both human and non-human portions, can be made using standard recombinant DNA techniques, and are within the scope of the present technology. For some uses, including in vivo use of the anti-PD-1 antibody of the present technology in humans as well as use of these agents in in vitro detection assays, it is possible to use chimeric or humanized anti-PD-1 antibodies. Such chimeric and humanized monoclonal antibodies can be produced by recombinant DNA techniques known in the art. Such useful methods include, e.g., but are not limited to, methods described in International Application No. PCT/US86/02269; U.S. Pat. No. 5,225,539; European Patent No. 184187; European Patent No. 171496; European Patent No. 173494; PCT International Publication No. WO 86/01533; U.S. Pat. Nos. 4,816,567; 5,225,539; European Patent No. 125023; Better, et al., 1988. Science 240: 1041-1043; Liu, et al., 1987. Proc. Natl. Acad. Sci. USA 84: 3439-3443; Liu, et al., 1987. J. Immunol. 139: 3521-3526; Sun, et al., 1987. Proc. Natl. Acad. Sci. USA 84: 214-218; Nishimura, et al., 1987. Cancer Res. 47: 999-1005; Wood, et al., 1985. Nature 314: 446-449; Shaw, et al., 1988. J. Natl. Cancer Inst. 80: 1553-1559; Morrison (1985) Science 229: 1202-1207; Oi, et al. (1986) BioTechniques 4: 214; Jones, et al., 1986. Nature 321: 552-525; Verhoeyan, et al., 1988. Science 239: 1534; Morrison, Science 229: 1202, 1985; Oi et al., BioTechniques 4: 214, 1986; Gillies et al., J. Immunol. Methods, 125: 191-202, 1989; U.S. Pat. No. 5,807,715; and Beidler, et al., 1988. J. Immunol. 141: 4053-4060. For example, antibodies can be humanized using a variety of techniques including CDR-grafting (EP 0 239 400; WO 91/09967; U.S. Pat. Nos. 5,530,101; 5,585,089; 5,859,205; 6,248,516; EP460167), veneering or resurfacing (EP 0 592 106; EP 0 519 596; Padlan E. A., Molecular Immunology, 28: 489-498, 1991; Studnicka et al., Protein Engineering 7: 805-814, 1994; Roguska et al., PNAS 91: 969-973, 1994), and chain shuffling (U.S. Pat. No. 5,565,332). In one embodiment, a cDNA encoding a murine anti-PD-1 monoclonal antibody is digested with a restriction enzyme selected specifically to remove the sequence encoding the Fc constant region, and the equivalent portion of a cDNA encoding a human Fc constant region is substituted (See Robinson et al., PCT/US86/02269; Akira et al., European Patent Application 184,187; Taniguchi, European Patent Application 171,496; Morrison et al., European Patent Application 173,494; Neuberger et al., WO 86/01533; Cabilly et al. U.S. Pat. No. 4,816,567; Cabilly et al., European Patent Application 125,023; Better et al. (1988) Science 240: 1041-1043; Liu et al. (1987) Proc. Natl. Acad. Sci. USA 84: 3439-3443; Liu et al. (1987) J Immunol 139: 3521-3526; Sun et al. (1987) Proc. Natl. Acad. Sci. USA 84: 214-218; Nishimura et al. (1987) Cancer Res 47: 999-1005; Wood et al. (1985) Nature 314: 446-449; and Shaw et al. (1988) J. Natl. Cancer Inst. 80: 1553-1559; U.S. Pat. Nos. 6,180,370; 6,300,064; 6,696,248; 6,706,484; 6,828,422.

In one embodiment, the present technology provides the construction of humanized anti-PD-1 antibodies that are unlikely to induce a human anti-mouse antibody (hereinafter referred to as “HAMA”) response, while still having an effective antibody effector function. As used herein, the terms “human” and “humanized”, in relation to antibodies, relate to any antibody which is expected to elicit a therapeutically tolerable weak immunogenic response in a human subject. In one embodiment, the present technology provides for a humanized anti-PD-1 antibodies, heavy and light chain immunoglobulins.

CDR Antibodies. In some embodiments, the anti-PD-1 antibody of the present technology is an anti-PD-1 CDR antibody. Generally the donor and acceptor antibodies used to generate the anti-PD-1 CDR antibody are monoclonal antibodies from different species; typically the acceptor antibody is a human antibody (to minimize its antigenicity in a human), in which case the resulting CDR-grafted antibody is termed a “humanized” antibody. The graft may be of a single CDR (or even a portion of a single CDR) within a single V_{H} or V_{L} of the acceptor antibody, or can be of multiple CDRs (or portions thereof) within one or both of the V_{H} and V_{L}. Frequently, all three CDRs in all variable domains of the acceptor antibody will be replaced with the corresponding donor CDRs, though one needs to replace only as many as necessary to permit adequate binding of the resulting CDR-grafted antibody to PD-1 protein. Methods for generating CDR-grafted and humanized antibodies are taught by Queen et al. U.S. Pat. Nos. 5,585,089; 5,693,761; 5,693,762; and Winter U.S. Pat. No. 5,225,539; and EP 0682040. Methods useful to prepare V_{H} and V_{L} polypeptides are taught by Winter et al., U.S. Pat. Nos. 4,816,397; 6,291,158; 6,291,159; 6,291,161; 6,545,142; EP 0368684; EP0451216; and EP0120694.

After selecting suitable framework region candidates from the same family and/or the same family member, either or both the heavy and light chain variable regions are produced by grafting the CDRs from the originating species into the hybrid framework regions. Assembly of hybrid antibodies or hybrid antibody fragments having hybrid variable chain regions with regard to either of the above aspects can be accomplished using conventional methods known to those skilled in the art. For example, DNA sequences encoding the hybrid variable domains described herein (i.e., frameworks based on the target species and CDRs from the originating species) can be produced by oligonucleotide synthesis and/or PCR. The nucleic acid encoding CDR regions can also be isolated from the originating species antibodies using suitable restriction enzymes and ligated into the target species framework by ligating with suitable ligation enzymes. Alternatively, the framework regions of the variable chains of the originating species antibody can be changed by site-directed mutagenesis.

Since the hybrids are constructed from choices among multiple candidates corresponding to each framework region, there exist many combinations of sequences which are amenable to construction in accordance with the principles described herein. Accordingly, libraries of hybrids can be assembled having members with different combinations of individual framework regions. Such libraries can be electronic database collections of sequences or physical collections of hybrids.

This process typically does not alter the acceptor antibody's FRs flanking the grafted CDRs. However, one skilled in the art can sometimes improve antigen binding affinity of the resulting anti-PD-1 CDR-grafted antibody by replacing certain residues of a given FR to make the FR more similar to the corresponding FR of the donor antibody. Suitable locations of the substitutions include amino acid residues adjacent to the CDR, or which are capable of interacting with a CDR (See, e.g., U.S. Pat. No. 5,585,089, especially columns 12-16). Or one skilled in the art can start with the donor FR and modify it to be more similar to the acceptor FR or a human consensus FR. Techniques for making these modifications are known in the art. Particularly if the resulting FR fits a human consensus FR for that position, or is at least 90% or more identical to such a consensus FR, doing so may not increase the antigenicity of the resulting modified anti-PD-1 CDR-grafted antibody significantly compared to the same antibody with a fully human FR.

Fc Modifications. In some embodiments, the anti-PD-1 antibodies of the present technology comprise a variant Fc region, wherein said variant Fc region comprises at least one amino acid modification relative to a wild-type Fc region (or the parental Fc region), such that said molecule has an altered affinity for an Fc receptor (e.g., an FcγR), provided that said variant Fc region does not have a substitution at positions that make a direct contact with Fc receptor based on crystallographic and structural analysis of Fc-Fc receptor interactions such as those disclosed by Sondermann et al., Nature, 406:267-273 (2000). Examples of positions within the Fc region that make a direct contact with an Fc receptor such as an FcγR, include amino acids 234-239 (hinge region), amino acids 265-269 (B/C loop), amino acids 297-299 (C7E loop), and amino acids 327-332 (F/G) loop.

In some embodiments, an anti-PD-1 antibody of the present technology has an altered affinity for activating and/or inhibitory receptors, having a variant Fc region with one or more amino acid modifications, wherein said one or more amino acid modification is a N297 substitution with alanine, or a K322 substitution with alanine.

Glycosylation Modifications. In some embodiments, anti-PD-1 antibodies of the present technology have an Fc region with variant glycosylation as compared to a parent Fc region. In some embodiments, variant glycosylation includes the absence of fucose; in some embodiments, variant glycosylation results from expression in GnT1-deficient CHO cells.

In some embodiments, the antibodies of the present technology, may have a modified glycosylation site relative to an appropriate reference antibody that binds to an antigen of interest (e.g., PD-1), without altering the functionality of the antibody, e.g., binding activity to the antigen. As used herein, “glycosylation sites” include any specific amino acid sequence in an antibody to which an oligosaccharide (i.e., carbohydrates containing two or more simple sugars linked together) will specifically and covalently attach.

Oligosaccharide side chains are typically linked to the backbone of an antibody via either N- or O-linkages. N-linked glycosylation refers to the attachment of an oligosaccharide moiety to the side chain of an asparagine residue. O-linked glycosylation refers to the attachment of an oligosaccharide moiety to a hydroxyamino acid, e.g., serine, threonine. For example, an Fc-glycoform (hPD-1-IgGln) that lacks certain oligosaccharides including fucose and terminal N-acetylglucosamine may be produced in special CHO cells and exhibit enhanced ADCC effector function.

In some embodiments, the carbohydrate content of an immunoglobulin-related composition disclosed herein is modified by adding or deleting a glycosylation site. Methods for modifying the carbohydrate content of antibodies are well known in the art and are included within the present technology, see, e.g., U.S. Pat. No. 6,218,149; EP 0359096B1; U.S. Patent Publication No. US 2002/0028486; International Patent Application Publication WO 03/035835; U.S. Patent Publication No. 2003/0115614; U.S. Pat. Nos. 6,218,149; 6,472,511; all of which are incorporated herein by reference in their entirety. In some embodiments, the carbohydrate content of an antibody (or relevant portion or component thereof) is modified by deleting one or more endogenous carbohydrate moieties of the antibody. In some certain embodiments, the present technology includes deleting the glycosylation site of the Fc region of an antibody, by modifying position 297 from asparagine to alanine.

Engineered glycoforms may be useful for a variety of purposes, including but not limited to enhancing or reducing effector function. Engineered glycoforms may be generated by any method known to one skilled in the art, for example by using engineered or variant expression strains, by co-expression with one or more enzymes, for example N-acetylglucosaminyltransferase III (GnTIII), by expressing a molecule comprising an Fc region in various organisms or cell lines from various organisms, or by modifying carbohydrate(s) after the molecule comprising Fc region has been expressed. Methods for generating engineered glycoforms are known in the art, and include but are not limited to those described in Umana et al., 1999, Nat. Biotechnol. 17: 176-180; Davies et al., 2001, Biotechnol. Bioeng. 74:288-294; Shields et al., 2002, J. Biol. Chem. 277:26733-26740; Shinkawa et al., 2003, J. Biol. Chem. 278:3466-3473; U.S. Pat. No. 6,602,684; U.S. patent application Ser. No. 10/277,370; U.S. patent application Ser. No. 10/113,929; International Patent Application Publications WO 00/61739A1; WO 01/292246A1; WO 02/311140A1; WO 02/30954A1; POTILLEGENT™ technology (Biowa, Inc. Princeton, N.J.); GLYCOMAB™ glycosylation engineering technology (GLYCART biotechnology AG, Zurich, Switzerland); each of which is incorporated herein by reference in its entirety. See, e.g., International Patent Application Publication WO 00/061739; U.S. Patent Application Publication No. 2003/0115614; Okazaki et al., 2004, JMB, 336: 1239-49.

Fusion Proteins. In one embodiment, the anti-PD-1 antibody of the present technology is a fusion protein. The anti-PD-1 antibodies of the present technology, when fused to a second protein, can be used as an antigenic tag. Examples of domains that can be fused to polypeptides include not only heterologous signal sequences, but also other heterologous functional regions. The fusion does not necessarily need to be direct, but can occur through linker sequences. Moreover, fusion proteins of the present technology can also be engineered to improve characteristics of the anti-PD-1 antibodies. For instance, a region of additional amino acids, particularly charged amino acids, can be added to the N-terminus of the anti-PD-1 antibody to improve stability and persistence during purification from the host cell or subsequent handling and storage. Also, peptide moieties can be added to an anti-PD-1 antibody to facilitate purification. Such regions can be removed prior to final preparation of the anti-PD-1 antibody. The addition of peptide moieties to facilitate handling of polypeptides are familiar and routine techniques in the art. The anti-PD-1 antibody of the present technology can be fused to marker sequences, such as a peptide which facilitates purification of the fused polypeptide. In select embodiments, the marker amino acid sequence is a hexa-histidine peptide (SEQ ID NO: 23), such as the tag provided in a pQE vector (QIAGEN, Inc., Chatsworth, Calif), among others, many of which are commercially available. As described in Gentz et al., Proc. Natl. Acad. Sci. USA 86: 821-824, 1989, for instance, hexa-histidine (SEQ ID NO: 23) provides for convenient purification of the fusion protein. Another peptide tag useful for purification, the “HA” tag, corresponds to an epitope derived from the influenza hemagglutinin protein. Wilson et al., Cell 37: 767, 1984.

Thus, any of these above fusion proteins can be engineered using the polynucleotides or the polypeptides of the present technology. Also, in some embodiments, the fusion proteins described herein show an increased half-life in vivo.

Fusion proteins having disulfide-linked dimeric structures (due to the IgG) can be more efficient in binding and neutralizing other molecules compared to the monomeric secreted protein or protein fragment alone. Fountoulakis et al., J. Biochem. 270: 3958-3964, 1995.

Similarly, EP-A-O 464 533 (Canadian counterpart 2045869) discloses fusion proteins comprising various portions of constant region of immunoglobulin molecules together with another human protein or a fragment thereof. In many cases, the Fc part in a fusion protein is beneficial in therapy and diagnosis, and thus can result in, e.g., improved pharmacokinetic properties. See EP-A 0232 262. Alternatively, deleting or modifying the Fc part after the fusion protein has been expressed, detected, and purified, may be desired. For example, the Fc portion can hinder therapy and diagnosis if the fusion protein is used as an antigen for immunizations. In drug discovery, e.g., human proteins, such as hPD-1, have been fused with Fc portions for the purpose of high-throughput screening assays to identify antagonists of hPD-1. Bennett et al., J. Molecular Recognition 8: 52-58, 1995; Johanson et al., J. Biol. Chem., 270: 9459-9471, 1995.

Labeled Anti-PD-1 antibodies. In one embodiment, the anti-PD-1 antibody of the present technology is coupled with a label moiety, i.e., detectable group. The particular label or detectable group conjugated to the anti-PD-1 antibody is not a critical aspect of the technology, so long as it does not significantly interfere with the specific binding of the anti-PD-1 antibody of the present technology to the PD-1 protein. The detectable group can be any material having a detectable physical or chemical property. Such detectable labels have been well-developed in the field of immunoassays and imaging. In general, almost any label useful in such methods can be applied to the present technology. Thus, a label is any composition detectable by spectroscopic, photochemical, biochemical, immunochemical, electrical, optical or chemical means. Labels useful in the practice of the present technology include magnetic beads (e.g., Dynabeads™), fluorescent dyes (e.g., fluorescein isothiocyanate, Texas red, rhodamine, and the like), radiolabels (e.g., ³H, ¹⁴C, ³⁵S, ¹²⁵I, ¹²¹I, ¹³¹I, ¹¹²In, ⁹⁹mTc), other imaging agents such as microbubbles (for ultrasound imaging), ¹⁸F, ¹¹C, ¹⁵O, ⁸⁹Zr (for Positron emission tomography), ^{99m}TC, ¹¹¹In (for Single photon emission tomography), enzymes (e.g., horse radish peroxidase, alkaline phosphatase and others commonly used in an ELISA), and calorimetric labels such as colloidal gold or colored glass or plastic (e.g., polystyrene, polypropylene, latex, and the like) beads. Patents that describe the use of such labels include U.S. Pat. Nos. 3,817,837; 3,850,752; 3,939,350; 3,996,345; 4,277,437; 4,275,149; and 4,366,241, each incorporated herein by reference in their entirety and for all purposes. See also Handbook of Fluorescent Probes and Research Chemicals (6^{th} Ed., Molecular Probes, Inc., Eugene OR.).

The label can be coupled directly or indirectly to the desired component of an assay according to methods well known in the art. As indicated above, a wide variety of labels can be used, with the choice of label depending on factors such as required sensitivity, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands can be used. Where a ligand has a natural anti-ligand, e.g., biotin, thyroxine, and cortisol, it can be used in conjunction with the labeled, naturally-occurring anti-ligands. Alternatively, any haptenic or antigenic compound can be used in combination with an antibody, e.g., an anti-PD-1 antibody.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidoreductases, particularly peroxidases. Fluorescent compounds useful as labeling moieties, include, but are not limited to, e.g., fluorescein and its derivatives, rhodamine and its derivatives, dansyl, umbelliferone, and the like. Chemiluminescent compounds useful as labeling moieties, include, but are not limited to, e.g., luciferin, and 2,3-dihydrophthalazinediones, e.g., luminol. For a review of various labeling or signal-producing systems which can be used, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it can be detected by exciting the fluorochrome with the appropriate wavelength of light and detecting the resulting fluorescence. The fluorescence can be detected visually, by means of photographic film, by the use of electronic detectors such as charge coupled devices (CCDs) or photomultipliers and the like. Similarly, enzymatic labels can be detected by providing the appropriate substrates for the enzyme and detecting the resulting reaction product. Finally, simple colorimetric labels can be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies, e.g., the anti-PD-1 antibodies. In this case, antigen-coated particles are agglutinated by samples comprising the target antibodies. In this format, none of the components need be labeled and the presence of the target antibody is detected by simple visual inspection.

### Methods of Using Anti-PD1 Antibodies of the Present Technology to Treat MMRd Rectal Cancer

In one aspect, the present disclosure provides a method for treating mismatch repair deficient (MMRd) rectal cancer in a patient in need thereof comprising administering to the patient an effective amount of an anti-PD1 antibody or an antigen binding fragment thereof, wherein the anti-PD1 antibody or antigen binding fragment comprises a heavy chain immunoglobulin variable domain (V_{H}) and a light chain immunoglobulin variable domain (V_{L}), wherein the V_{H} comprises a V_{H}-CDR1 sequence of SEQ ID NO: 5, a V_{H}-CDR2 sequence of SEQ ID NO: 6, and a V_{H}-CDR3 sequence of SEQ ID NO: 7 and the V_{L} comprises a V_{L}-CDR1 sequence of SEQ ID NO: 8, a V_{L}—CDR2 sequence of SEQ ID NO: 9, and a V_{L}-CDR3 sequence of SEQ ID NO: 10, and wherein the patient has not received a prior cancer therapy. The antigen binding fragment may be selected from the group consisting of Fab, F(ab′)2, Fab′, scFv, and Fv. Examples of prior cancer therapy include immunotherapy, chemotherapy, or radiation. In certain embodiments, the chemotherapy comprises one or more of fluoropyrimidine, leucovorin calcium (folinic acid), fluorouracil, capecitabine, and oxaliplatin.

In some embodiments, the V_{H} comprises the sequence of SEQ ID NO: 3 and the V_{L} comprises the sequence of SEQ ID NO: 1. Additionally or alternatively, in some embodiments, the anti-PD-1 antibody or antigen binding fragment comprises a heavy chain (HC) amino acid sequence comprising SEQ ID NO: 4 and a light chain (LC) amino acid sequence comprising SEQ ID NO: 2. The MMRd rectal cancer may be stage II or stage III. In other embodiments, the MMRd rectal cancer is node-positive (e.g., spread to lymph nodes) or node-negative. In any and all embodiments of the methods disclosed herein, the MMRd rectal cancer has a tumor stage selected from the group consisting of T1, T2, T3 and T4.

Additionally or alternatively, in some embodiments of the methods disclosed herein, the MMRd rectal cancer is locally advanced rectal cancer. The MMRd rectal cancer may comprise a deficiency in one or more of MLH1, MSH2, MSH6 and PMS2. In any of the preceding embodiments of the methods disclosed herein, MMR deficiency of the MMRd rectal cancer is determined by immunohistochemistry. Additionally or alternatively, in certain embodiments, the MMRd rectal cancer comprises a somatic MMR mutation selected from the group consisting of MSH2 c.1165C>T, MSH2 c.1204C>T, MSH2 c.1061delA, MSH2 c.1650dupA, MSH2 c.363dupT, MSH2 c.1413_1420delACCT TCAT, MSH6 c.2319_2320delCC, MSH6 c.2319_2337delinsTA, MSH6 c2323_2337delAAGCAATGGCTTTGT (SEQ ID NO: 22), MSH6 c.643G>A, MLH1 c.469delT, and MLH1 c.1420_1426delCGGGAAG.

In any and all embodiments of the methods disclosed herein, the patient is diagnosed with Lynch Syndrome. The patient may exhibit rectal bleeding, constipation, and/or abdominal pain prior to administration of the anti-PD1 antibody or antigen binding fragment. In some embodiments, the patient comprises a germline pathogenic variant selected from the group consisting of MSH2 c.687delA, MSH2 c.8942+3A>T, MSH2 c.942+3A>T, MSH6 c.1969delC, MSH2 c.1784T>G, PMS2 c.2500_2501delinsG, MLH1 c.1489dupC, and MSH6 c.3476dupA. Additionally or alternatively, in some embodiments, the patient does not comprise a BRAF V600E mutation and/or comprises tumors having a tumor mutation burden (TMB) ranging from of 30-95 mutations per Megabase. In certain embodiments, the TMB is about 30-35, about 35-40, about 40-45, about 45-50, about 50-55, about 55-60, about 60-65, about 65-70, about 70-75, about 75-80, about 80-85, about 85-90, or about 90-95 mutations per Megabase.

Additionally or alternatively, in some embodiments, the anti-PD1 antibody or antigen binding fragment is administered intravenously, intramuscularly, intraperitoneally, subcutaneously, rectally, parenterally, or intradermally. In any of the preceding embodiments of the methods disclosed herein, the anti-PD1 antibody or antigen binding fragment is administered once per every two weeks, once per every three weeks, or once a month. In certain embodiments, the anti-PD1 antibody or antigen binding fragment is administered for at least 2 months, at least 3 months, at least 4 months, at least 5 months, at least 6 months, at least 7 months, at least 8 months, at least 9 months, at least 10 months, at least 11 months, at least 1 year, or at least 2 or more years. In any and all embodiments of the methods disclosed herein, the patient exhibits endoscopic complete response (CR) and/or radiographic CR after administration of the anti-PD1 antibody or antigen binding fragment.

The compositions of the present technology may optionally be administered as a single bolus to a subject in need thereof. Alternatively, the dosing regimen may comprise multiple administrations performed at various times after the appearance of tumors.

Administration can be carried out by any suitable route, including orally, intranasally, parenterally (intravenously, intramuscularly, intraperitoneally, or subcutaneously), rectally, intracranially, intratumorally, intrathecally, or topically. Administration includes self-administration and the administration by another. It is also to be appreciated that the various modes of treatment of medical conditions as described are intended to mean “substantial”, which includes total but also less than total treatment, and wherein some biologically or medically relevant result is achieved.

In any and all embodiments of the methods disclosed herein, the methods of the present technology further comprise sequentially, simultaneously or separately administering to the patient an effective amount of an additional therapy (e.g., chemotherapy, radiotherapy, surgery).

In some embodiments, the antibodies of the present technology comprise pharmaceutical formulations which may be administered to subjects in need thereof in one or more doses (e.g., 1, 2, 3, 4, 5, 6, 7, 8, 9 or more doses). Dosage regimens can be adjusted to provide the desired response (e.g., a therapeutic response).

Typically, an effective amount of the antibody compositions of the present technology, sufficient for achieving a therapeutic effect, range from about 0.000001 mg per kilogram body weight per day to about 10,000 mg per kilogram body weight per day. Typically, the dosage ranges are from about 0.0001 mg per kilogram body weight per day to about 100 mg per kilogram body weight per day. For administration of anti-PD-1 antibodies, the dosage ranges from about 0.0001 to 100 mg/kg, and more usually 0.01 to 5 mg/kg every week, every two weeks or every three weeks, of the subject body weight. For example, dosages can be 1 mg/kg body weight or 10 mg/kg body weight every week, every two weeks or every three weeks or within the range of 1-10 mg/kg every week, every two weeks or every three weeks. In one embodiment, a single dosage of antibody ranges from 0.1-10,000 micrograms per kg body weight. In one embodiment, antibody concentrations in a carrier range from 0.2 to 2000 micrograms per delivered milliliter. An exemplary treatment regime entails administration once per every two-three weeks or once a month or once every 3 to 6 months. Anti-PD-1 antibodies may be administered on multiple occasions. Intervals between single dosages can be hourly, daily, weekly, monthly or yearly. Intervals can also be irregular as indicated by measuring blood levels of the antibody in the subject. In some methods, dosage is adjusted to achieve a serum antibody concentration in the subject of from about 75 μg/mL to about 125 μg/mL, 100 μg/mL to about 150 μg/mL, from about 125 μg/mL to about 175 μg/mL, or from about 150 μg/mL to about 200 μg/mL. Alternatively, anti-PD-1 antibodies can be administered as a sustained release formulation, in which case less frequent administration is required. Dosage and frequency vary depending on the half-life of the antibody in the subject. The dosage and frequency of administration can vary depending on whether the treatment is prophylactic or therapeutic. In prophylactic applications, a relatively low dosage is administered at relatively infrequent intervals over a long period of time. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

Toxicity. Optimally, an effective amount (e.g., dose) of an anti-PD-1 antibody described herein will provide therapeutic benefit without causing substantial toxicity to the subject. Toxicity of the anti-PD-1 antibody described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the anti-PD-1 antibody described herein lies within a range of circulating concentrations that include the effective dose with little or no toxicity. The dosage can vary within this range depending upon the dosage form employed and the route of administration utilized. The exact formulation, route of administration and dosage can be chosen by the individual physician in view of the subject's condition. See, e.g., Fingl et al., In: The Pharmacological Basis of Therapeutics, Ch. 1 (1975).

Formulations of Pharmaceutical Compositions. According to the methods of the present technology, the anti-PD-1 antibody can be incorporated into pharmaceutical compositions suitable for administration. The pharmaceutical compositions generally comprise recombinant or substantially purified antibody and a pharmaceutically-acceptable carrier in a form suitable for administration to a subject. Pharmaceutically-acceptable carriers are determined in part by the particular composition being administered, as well as by the particular method used to administer the composition. Accordingly, there is a wide variety of suitable formulations of pharmaceutical compositions for administering the antibody compositions (See, e.g., Remington's Pharmaceutical Sciences, Mack Publishing Co., Easton, PA 18^{th} ed., 1990). The pharmaceutical compositions are generally formulated as sterile, substantially isotonic and in full compliance with all Good Manufacturing Practice (GMP) regulations of the U.S. Food and Drug Administration.

The terms “pharmaceutically-acceptable,” “physiologically-tolerable,” and grammatical variations thereof, as they refer to compositions, carriers, diluents and reagents, are used interchangeably and represent that the materials are capable of administration to or upon a subject without the production of undesirable physiological effects to a degree that would prohibit administration of the composition. For example, “pharmaceutically-acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous. “Pharmaceutically-acceptable salts and esters” means salts and esters that are pharmaceutically-acceptable and have the desired pharmacological properties. Such salts include salts that can be formed where acidic protons present in the composition are capable of reacting with inorganic or organic bases. Suitable inorganic salts include those formed with the alkali metals, e.g., sodium and potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically-acceptable esters include esters formed from carboxy, sulfonyloxy, and phosphonoxy groups present in the anti-PD-1 antibody, e.g., C₁₋₆ alkyl esters. When there are two acidic groups present, a pharmaceutically-acceptable salt or ester can be a mono-acid-mono-salt or ester or a di-salt or ester; and similarly where there are more than two acidic groups present, some or all of such groups can be salified or esterified. An anti-PD-1 antibody named in this technology can be present in unsalified or unesterified form, or in salified and/or esterified form, and the naming of such anti-PD-1 antibody is intended to include both the original (unsalified and unesterified) compound and its pharmaceutically-acceptable salts and esters. Also, certain embodiments of the present technology can be present in more than one stereoisomeric form, and the naming of such anti-PD-1 antibody is intended to include all single stereoisomers and all mixtures (whether racemic or otherwise) of such stereoisomers. A person of ordinary skill in the art, would have no difficulty determining the appropriate timing, sequence and dosages of administration for particular drugs and compositions of the present technology.

Examples of such carriers or diluents include, but are not limited to, water, saline, Ringer's solutions, dextrose solution, and 5% human serum albumin. Liposomes and non-aqueous vehicles such as fixed oils may also be used. The use of such media and compounds for pharmaceutically active substances is well known in the art. Except insofar as any conventional media or compound is incompatible with the anti-PD-1 antibody, use thereof in the compositions is contemplated. Supplementary active compounds can also be incorporated into the compositions.

A pharmaceutical composition of the present technology is formulated to be compatible with its intended route of administration. The anti-PD-1 antibody compositions of the present technology can be administered by parenteral, topical, intravenous, oral, subcutaneous, intraarterial, intradermal, transdermal, rectal, intracranial, intrathecal, intraperitoneal, intranasal; or intramuscular routes, or as inhalants. The anti-PD-1 antibody can optionally be administered in combination with other agents that are at least partly effective in treating MMRd rectal cancer (e.g., locally advanced rectal cancer).

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. In many cases, it will be desirable to include isotonic compounds, e.g., sugars, polyalcohols such as manitol, sorbitol, sodium chloride in the composition. Prolonged absorption of the injectable compositions can be brought about by including in the composition a compound which delays absorption, e.g., aluminum monostearate and gelatin.

Sterile injectable solutions can be prepared by incorporating an anti-PD-1 antibody of the present technology in the required amount in an appropriate solvent with one or a combination of ingredients enumerated above, as required, followed by filtered sterilization. Generally, dispersions are prepared by incorporating the anti-PD-1 antibody into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof. The antibodies of the present technology can be administered in the form of a depot injection or implant preparation which can be formulated in such a manner as to permit a sustained or pulsatile release of the active ingredient.

Oral compositions generally include an inert diluent or an edible carrier. They can be enclosed in gelatin capsules or compressed into tablets. For the purpose of oral therapeutic administration, the anti-PD-1 antibody can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel, or corn starch; a lubricant such as magnesium stearate or Sterotes; a glidant such as colloidal silicon dioxide; a sweetening compound such as sucrose or saccharin; or a flavoring compound such as peppermint, methyl salicylate, or orange flavoring.

For administration by inhalation, the anti-PD-1 antibody is delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of nasal sprays or suppositories. For transdermal administration, the anti-PD-1 antibody is formulated into ointments, salves, gels, or creams as generally known in the art.

The anti-PD-1 antibody can also be prepared as pharmaceutical compositions in the form of suppositories (e.g., with conventional suppository bases such as cocoa butter and other glycerides) or retention enemas for rectal delivery.

In one embodiment, the anti-PD-1 antibody is prepared with carriers that will protect the anti-PD-1 antibody against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically-acceptable carriers. These can be prepared according to methods known to those skilled in the art, e.g., as described in U.S. Pat. No. 4,522,811.

### Kits of the Present Technology

The present technology provides kits for the treatment of MMRd rectal cancer (e.g., locally advanced rectal cancer) comprising at least one immunoglobulin-related composition of the present technology (e.g., any antibody or antigen binding fragment described herein), or a functional variant (e.g., substitutional variant) thereof. Optionally, the above described components of the kits of the present technology are packed in suitable containers and labeled for treatment of MMRd rectal cancer (e.g., locally advanced rectal cancer). The above-mentioned components may be stored in unit or multi-dose containers, for example, sealed ampoules, vials, bottles, syringes, and test tubes, as an aqueous, preferably sterile, solution or as a lyophilized, preferably sterile, formulation for reconstitution. The kit may further comprise a second container which holds a diluent suitable for diluting the pharmaceutical composition towards a higher volume. Suitable diluents include, but are not limited to, the pharmaceutically acceptable excipient of the pharmaceutical composition and a saline solution. Furthermore, the kit may comprise instructions for diluting the pharmaceutical composition and/or instructions for administering the pharmaceutical composition, whether diluted or not. The containers may be formed from a variety of materials such as glass or plastic and may have a sterile access port (for example, the container may be an intravenous solution bag or a vial having a stopper which may be pierced by a hypodermic injection needle). The kit may further comprise more containers comprising a pharmaceutically acceptable buffer, such as phosphate-buffered saline, Ringer's solution and dextrose solution. It may further include other materials desirable from a commercial and user standpoint, including other buffers, diluents, filters, needles, syringes, culture medium for one or more of the suitable hosts. The kits may optionally include instructions customarily included in commercial packages of therapeutic or diagnostic products, that contain information about, for example, the indications, usage, dosage, manufacture, administration, contraindications and/or warnings concerning the use of such therapeutic or diagnostic products.

The kit can also comprise, e.g., a buffering agent, a preservative or a protein-stabilizing agent. Each component of the kit can be enclosed within an individual container and all of the various containers can be within a single package, along with instructions for using the kit. The kits of the present technology may contain a written product on or in the kit container. The written product describes how to use the reagents contained in the kit, e.g., for treatment of MMRd rectal cancer (e.g., locally advanced rectal cancer) in a subject in need thereof. In certain embodiments, the use of the reagents can be according to the methods of the present technology.

### EXAMPLES

The present technology is further illustrated by the following Examples, which should not be construed as limiting in any way. The examples herein are provided to illustrate advantages of the present technology and to further assist a person of ordinary skill in the art with preparing or using the compositions and systems of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects or embodiments of the present technology.

### Example 1: Materials and Methods

Patients.

Patients were eligible for enrollment if they were 18 years of age or older and had mismatch repair-deficient stage II or stage III rectal cancer that had been diagnosed on the basis of standard clinical criteria. Mismatch-repair status was determined with the use of a chromogenic immunohistochemical assay for the detection of loss of expression of MLH1, MSH1, MSH6, and PMS2. Staging was confirmed by standard magnetic resonance imaging (MRI), which was performed according to a specified protocol for rectal cancer; computed tomography (CT) of the chest, abdomen, and pelvis; and colonoscopy. Positron-emission tomography (PET) was performed. Patients were required to have an Eastern Cooperative Oncology Group (ECOG) performance-status score of 0 or 1 (on a 5-point scale, with higher numbers indicating greater disability) and no evidence of distant metastases. Other key eligibility criteria included no previous receipt of immunotherapy, chemotherapy, or radiation for the rectal tumor and no active autoimmune disease, active infectious disease, or recent receipt (within the previous 7 days) of immunosuppressive therapy.

Study Design.

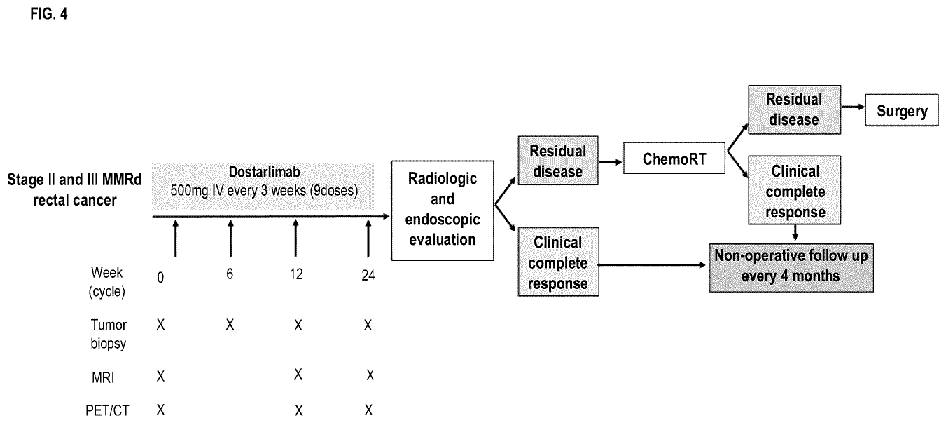

In this single-group, prospective phase 2 study, neoadjuvant dostarlimab administered intravenously at a dose of 500 mg every 3 weeks for 6 months (nine cycles) was to be followed by standard radiation therapy (total dose of 5040 cGy given in 28 fractions) with concurrent administration of capecitabine at standard doses and then total mesorectal excision. Patients who had a clinical complete response (as defined below) after completion of either induction anti-PD-1 therapy or chemoradiotherapy subsequently underwent nonoperative follow-up (FIG. 4).

Patients were assessed for clinical response with the use of endoscopic and digital rectal examinations at baseline (before treatment), at 6 weeks, at 3 months, and at 6 months and then every 4 months after the start of treatment. T2-weighted and diffusion-weighted MRI of the rectum, ¹⁸F-fluorodeoxyglucose (FDG)-PET, and CT of the chest, abdomen, and pelvis were performed at baseline, at 3 months, and at 6 months and then every 4 months after the start of treatment. Tumor biopsies were performed at the time of each endoscopy. All assessments were to be performed early if patients had clinical symptoms of progression.

Tumor response was determined on the basis of T2-weighted and diffusion-weighted MRI of the rectum, endoscopic evaluation, and digital rectal examination. A clinical complete response was defined as the absence of residual disease on digital and endoscopic rectal examination, as well as the absence of residual disease on rectal MRI, with no restricted diffusion on T2-weighted imaging.

Clinical complete response (cCR) includes both endoscopic complete response (CR) and radiographic CR. Endoscopic CR is defined by disappearance of the rectal primary tumors and a normal digital rectal exam. Radiographic CR (as determined by Rectal MRI) is defined by lack of signal at DWI with scar on T2WI (DWI volume=0) and each target lymph node having decreased short axis to <0.5 cm.

Endpoints.

The study is evaluating two primary end points, with a planned enrollment of 30 patients. One end point is sustained clinical complete response 12 months after completion of dostarlimab therapy (in patients who do not undergo surgery) or pathological complete response (in patients who undergo surgery) after completion of dostarlimab therapy with or without chemoradiotherapy. Pathological complete response was defined in the protocol as the absence of residual cancer on the histologic examination of surgical specimens. The other end point is overall response to neoadjuvant dostarlimab therapy with or without chemoradiotherapy. Only the second end point is reported here. Overall response was determined on the basis of T2-weighted and diffusion-weighted MRI of the rectum, endoscopic visualization, and digital rectal examination. Overall response was defined in the protocol as progressive disease, stable disease, partial response, near-complete response, or complete response.

Pathological and Genomic Analyses.

Formalin-fixed, paraffin-embedded tumor samples obtained from biopsies that were performed during the study were stained with hematoxylin and eosin and reviewed by a trained pathologist, who visually assessed the samples to confirm the diagnosis, identify the general histologic features, and estimate the percentage of viable tumor cells in each tumor sample. Mismatch-repair status was determined with the use of a chromogenic immunohistochemical assay for the detection of loss of expression of MLH1, MSH1, MSH6, and PMS2.

Tumor-specific and germline comprehensive genomic analyses were performed with the use of Memorial Sloan Kettering-Integrated Mutation Profiling of Actionable Cancer Targets (MSK-IMPACT), a next-generation sequencing assay. This assay, which has been approved by the Food and Drug Administration, detects somatic and germline genomic alterations in more than 400 genes and assesses tumor mutational burden. All the patients undergoing comprehensive molecular analysis for somatic tumor-specific alterations or analysis of known hereditary alterations provided additional written informed consent specific to those analyses.

Formalin-fixed, paraffin-embedded biopsy sections were also evaluated with the use of quantitative immunofluorescence analysis that was standardized for simultaneous measurement of DAPI (4′,6-diamidine-2-phenylindole) for all cells, cytokeratin for tumor and normal gut epithelial cells (clone AE1/AE3, Dako), CD20+B lymphocytes (clone L26, M0755; Dako), programmed death ligand 1 (PD-L1) (clone E1L3N, CST), and CD8+T lymphocytes (clone C8/144B, M7103; Dako). The marker levels were measured in selected tissue compartments and expressed as quantitative immunofluorescence scores on the basis of arbitrary units of fluorescence.

Statistical Analysis.

The overall response rate was assessed using a one-sample hypothesis; the null hypothesis to be tested was that the percentage of patients with an overall response would be less than 25%. Successful rejection of the null hypothesis would require 6 or more patients with an overall response by the end of the first stage (after 15 patients had been enrolled) and 11 or more patients with an overall response by the end of the second stage (after 30 patients had been enrolled). This decision rule would result in a type I error rate of 6% if 25% of the patients had an overall response and would provide the study with 84% power if 50% had an overall response. The null hypothesis was established on the basis of a study by Seligmann et al., in which the observed response to chemotherapy among patients with mismatch repair-deficient rectal cancers was 7% (8 of 115 patients). The results are reported without awaiting full enrollment, because the second criterion for the decision rule (>11 patients having an overall response) has already been met.

Binomial proportions are reported with a 95% exact confidence interval. Quantitative immunofluorescence scores for pathological samples obtained at baseline, during treatment, and during follow-up were compared with the use of the Mann-Whitney test. The statistical analysis and graphical representation were performed with GraphPad Prism software, version 9.0.2 (GraphPad Software). P values of less than 0.05 were considered to indicate statistical significance.

Pathological Assessments.

Analysis of IHC expression of MMR proteins was performed by using a clinically validated standard procedure with a polymer-based detection system. Primary monoclonal antibodies used were MLH1 (clone ES05, 1:500 dilution, Leica Biosystems/Novocastra), MSH2 (clone G219-1129, 1:750 dilution, Cell Marque), MSH6 (clone EP49, 1:500 dilution, Agilent/Dako), PMS2 (clone A16-4, diluted 1:200; BD Biosciences).

Non-neoplastic colonic mucosa and colorectal tumors known to be deficient of MLH1, MSH2, MSH6, and PMS2 were used as external positive and negative controls, respectively. Retained expression of each protein was defined by nuclear IHC reactivity of tumor cells, whereas loss of expression for each protein was defined by absence of nuclear IHC reactivity of tumor cells. Tumors were MMR proficient if all four proteins were expressed (retained) by IHC and MMR deficient (MMRd) if any of the four proteins was not expressed (lost) by IHC.

Formalin-fixed, paraffin-embedded (FFPE) tumor samples from biopsies conducted on trial patients were stained with hematoxylin and eosin (H&E) and scanned using the Aperio AT2 Digital Whole Slide Scanner (Leica Biosystems). The H&E images were reviewed by a trained pathologist, who visually assessed at low (lx original magnification) and high-power fields (˜400×original magnification) to assess the general histologic features and estimate the percentage of viable tumor cells and tumor infiltrating lymphocytes (TILs) in each tumor sample using 5% increments.

A Multiplexed Quantitative Immunofluorescence (QIF) panel was standardized for simultaneous measurement of DAPI (all cells), cytokeratin for tumor and normal gut epithelial cells (AE1/AE3, Dako), CD20-positive B lymphocytes (clone L26, M0755, Dako), PD-L1 (E1L3N, CST), and CD8 positive T-cells (clone C8/144B, M7103, Dako). The QIF staining was conducted using a previous reported protocol. Briefly, FFPE tissue sections were deparaffinized and antigen retrieval was performed in pH8, 1 mM EDTA solution (Sigma Aldrich) and heated at 97° C. (PT module, Lab Vision, Thermo Scientific) for 20 minutes. Inactivation of endogenous peroxidase was done with Ready-to-Use Dual Endogenous Enzyme Block (S2003, Dako) for 10 minutes. The slides were then incubated with blocking solution containing 0.3% BSA and 0.05% Tween-20 in Tris Buffered Solution (TBS) for 30 minutes. Primary antibodies were incubated for 1 hour at room temperature. Secondary antibodies were isotype-specific HRP-conjugated and signal detection was achieved by tyramide-based amplification. Secondary antibodies included anti-rabbit IgG Envision (K4009, Dako) with Cy5-tyramide (PerkinElmer), anti-mouse IgG1 antibody (clone M114D12, eBioscience) with biotinylated tyramide/Streptavidine-Alexa750 conjugate (PerkinElmer), and anti-mouse IgG2a antibody (ab97245, Abcam) with Cy3-tyramide (PerkinElmer). Cytokeratin was detected with anti-rabbit pan-cytokeratin Alexa 488-conjugated (Dako) in 0.3% BSA and 0.05% Tween-20 in TBS. Nuclei were detected with DAPI. The residual HRP activity between antibody incubations was eliminated using a solution containing 100 mM benzoic hydrazide and 50 mM hydrogen peroxide in phosphate buffered solution for 7 minutes, applied twice. The quantitative measurement of the fluorescent signal was conducted using the AQUA® method of QIF as reported, allowing the selective measurement of targets in marker defined tissue compartments. Semi-quantitative estimation of the PD-L1 protein levels was based on the distribution of scores within the cohort and stratified using tertiles. Comparisons between QIF scores across groups were conducted using the Mann-Whitney test. The statistical analysis and graphical representation were performed in GraphPad Prism v. 9.0.2 for Windows (GraphPad Software, Inc., San Diego, CA). All two-tailed P-values ≤0.05 were considered statistically significant.

Sequencing Analysis.

Formalin-fixed, paraffin-embedded tumor samples and matched normal blood samples were analyzed in a Clinical Laboratory Improvement Amendments (CLIA)-certified molecular laboratory using MSK-IMPACT, a capture-based next-generation sequencing platform. The MSK-IMPACT assay achieves high depth of sequencing (800×) and can detect mutations, copy number alterations, and select rearrangements in 505 cancer-associated genes, as well as assess MSI status.

Germline Analysis.

Prospective secondary germline analysis was offered to patients who consented to tumor genetic analysis utilizing an IRB-approved protocol. Germline analysis using blood-derived DNA included a 76- to 88-gene MSK-IMPACT panel including all cancer-predisposing genes inclusive of likely pathogenic and pathogenic variants in the MMR genes associated with Lynch syndrome, identified by the American College of Medical Genetics and Genomics guidelines, in a Clinical Laboratory Improvement Amendments (CLIA)-approved lab. Inclusive of likely pathogenic and pathogenic variants in the MMR genes associated with Lynch syndrome.

### Example 2: Dostarlimab Therapy Leads to Complete Response in all MMRd Colorectal Cancer Patients

Patient Characteristics

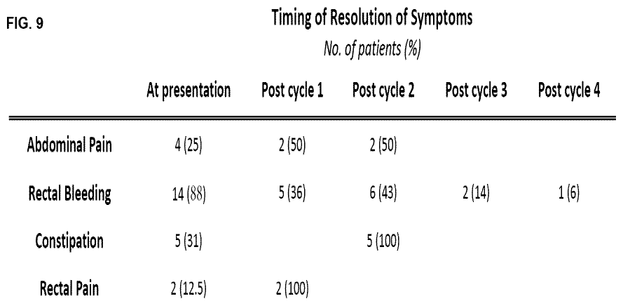

A total of 16 patients have been enrolled and treated (Table 1). Of these patients, 12 have been enrolled for longer than 6 months and have completed the nine planned cycles (6 months) of dostarlimab. The median follow-up time from study enrollment to the clinical data cutoff for the 12 patients is 12 months (range, 6 to 25). The remaining 4 patients have received at least one dose of dostarlimab and continue to receive treatment. The median age of all the enrolled patients is 54 years (range, 26 to 78), and 62% are women. All 16 patients met the eligibility criteria, and no patients have withdrawn from the study. Of the 16 patients, 15 have clinical stage III disease, and 1 has clinical stage II disease. The most common presenting symptoms were rectal bleeding (in 88% of the patients), constipation (in 310%), and abdominal pain (in 250%) (FIG. 9).

TABLE 1
Demographics

| | | |
|---|---|---|
| Characteristic | Number (N) | % |

| Total enrolled | 16 | | 100 |
|---|---|---|---|
| Sex | | | |
| Male | 6 | | 38 |
| Female | 10 | | 63 |
| Age, median (range) | 54 | (26, 77) | |

| | | |
|---|---|---|
| Race | | |
| White | 11 | 69 |
| Asian/Far East/Indian | 3 | 19 |
| Subcontinent | | |
| Black or African American | 2 | 12 |
| Ethnicity | | |
| Not Hispanic or Latino | 15 | 94 |
| Hispanic or Latino | 1 | 6 |
| ECOG | | |

| 0 | 12 | | 75 |
|---|---|---|---|
| 1 | 4 | | 25 |
| Tumor Staging | | | |
| T1/2 | 4 | | 25 |
| T3 | 9 | | 56 |
| T4 | 3 | | 19 |
| Nodal Staging | | | |
| Node-positive | 15 | | 94 |
| Node-negative | 1 | | 6 |
| Distance from Anal Verge (cm), | 5 | (0.9, 8.9) | |

median (range)

Tumor Characteristics

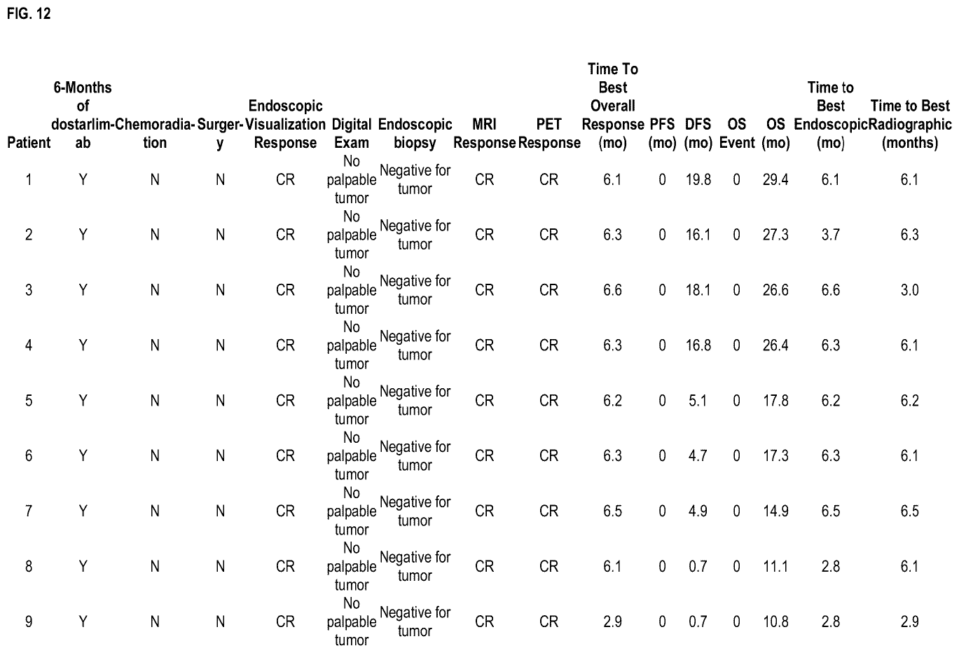

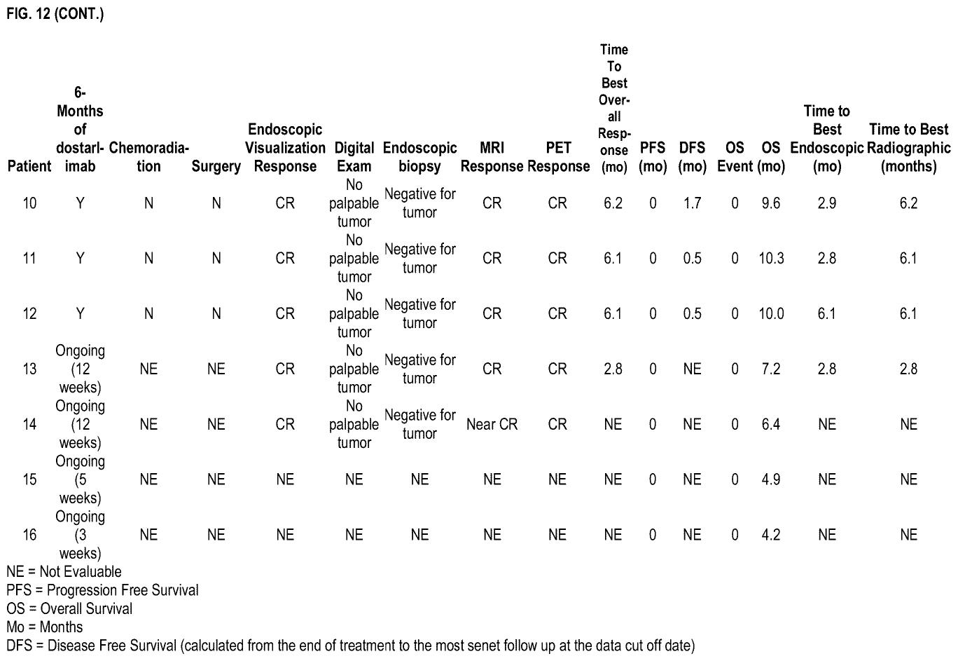

Mutational analysis of the tumor specimens by NGS confirmed microsatellite instability in all cases tested (n=14) and demonstrated a high tumor mutation burden with a range of 37.9-93.9 mut/Mb (average 68.6 mut/Mb). BRA V600E mutations were absent in all cases tested (n=14) (Table 2, FIGS. 11-12).

None of the patients had a known family history of Lynch Syndrome. Germline analysis identified pathogenic genomic alterations in 57% (8/14) of patients, which were all associated with Lynch syndrome. Alterations in MSH2 were most prominent in 4 of the 8 cases, but pathogenic alterations in MSH6, MLH1 and PMS2 were also present (Table 2).

TABLE 2

| Individual patient data | | | | | | | | | | |
|---|---|---|---|---|---|---|---|---|---|---|
| | | | | | Germline | | | | | |
| | | | | | Pathogenic | | | | BRAF | |
| | | | Stage | Stage | Variant (Lynch | | | | V600E | |
| Patient | Gender | Age | T | N | Syndrome) | MMR IHC | PD-L1 | TILs | mutation | TMB |
| 1 | F | 38 | T4 | N+ | MSH2 | MSH2, MSH6 | + | +++ | WT | 88.6 |
| | | | | | (c.687delA) | absent | | | | |
| 2 | F | 30 | T3 | N+ | MSH2 | MSH2, MSH6 | ++ | + | WT | 45.6 |
| | | | | | (c.8942 + 3A > T) | absent | | | | |
| 3 | F | 61 | T1/2 | N+ | None | MSH2, MSH6 | +++ | +++ | WT | 62.3 |
| | | | | | | absent | | | | |
| 4 | F | 28 | T4 | N+ | None | MSH2, MSH6 | + | ++ | WT | 65.0 |
| | | | | | | absent | | | | |
| 5 | F | 53 | T1/2 | N+ | MSH2 | MSH2 absent | + | + | WT | 103.0 |
| | | | | | (c.942 + 3A > T) | | | | | |
| 6 | F | 77 | T1/2 | N+ | MSH6 | MSH6 absent | +++ | +++ | WT | 93.9 |
| | | | | | (c. 1969delC) | | | | | |
| 7 | F | 77 | T1/2 | N+ | None | MLH1 and | ++ | ++ | WT | 75.0 |
| | | | | | | PMS2 absent | | | | |
| 8 | F | 55 | T3 | N+ | MSH2 | MSH2 absent | ++ | + | WT | 78.3 |
| | | | | | (c.1784T > G) | | | | | |
| 9 | M | 68 | T3 | N+ | None | MSH2 and | +++ | ++ | WT | 62.6 |
| | | | | | | MSH6 absent | | | | |
| 10 | F | 78 | T3 | N− | None | MLH1 and | + | + | WT | 37.9 |
| | | | | | | PMS2 absent | | | | |
| 11 | F | 55 | T3 | N+ | PMS2 (c.2500_ | MSH2, MSH 6 | ++ | ++ | WT | 52.7 |
| | | | | | 2501delinsG) | absent | | | | |
| 12 | M | 27 | T3 | N+ | None | PMS2 absent | +++ | +++ | WT | 54.4 |
| 13 | M | 26 | T3 | N+ | MLH1 | MLH1 and | NA | NA | WT | 47.8 |
| | | | | | (c.1489dupC) | PMS2 absent | | | | |
| 14 | M | 43 | T3 | N+ | MSH6 | MSH6 absent | NA | NA | WT | 74.1 |
| | | | | | (c.3476dupA) | | | | | |
| 15 | M | 59 | T3 | N+ | NA | PMS2 absent | NA | NA | NA | NA |
| 16 | M | 51 | T4b | N+ | NA | MSH2 absent | NA | NA | NA | NA |

NA = Not available
NE = Not evaluable

Primary Objectives and Efficacy

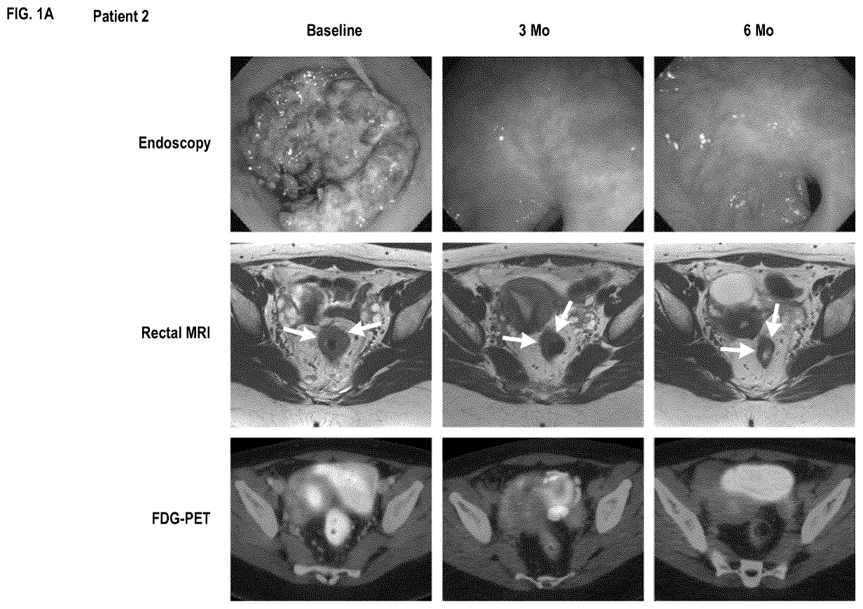

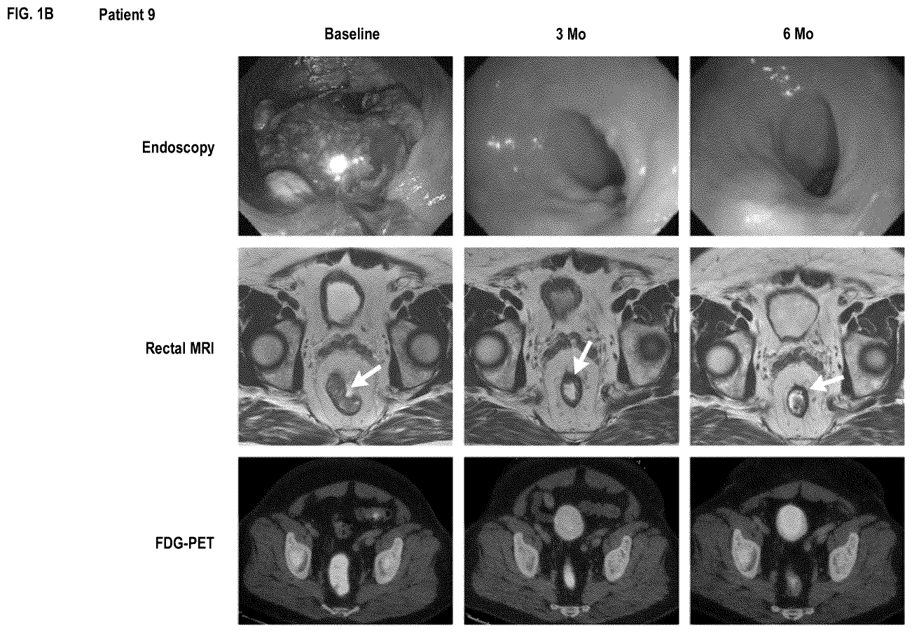

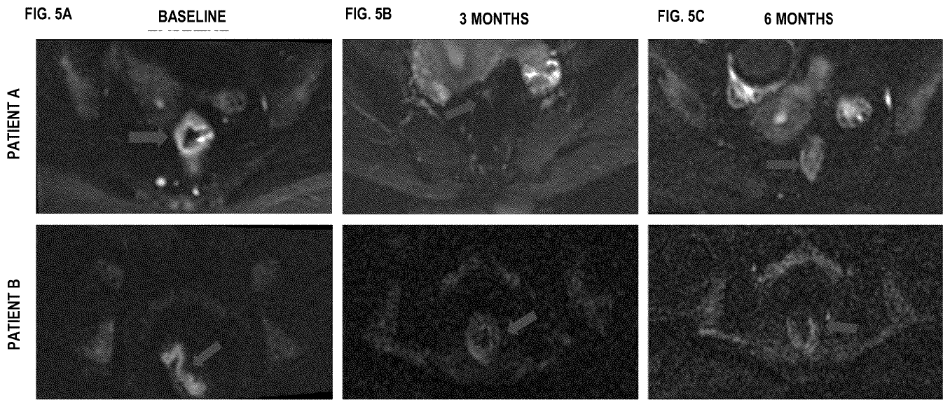

The co-primary endpoint of overall response rate was met. The response rate was 100% (95% CI: 74-100%) in 12 consecutive patients who have completed 6 months of therapy (FIGS. 1A-1B, Table 2, FIGS. 5A-5C). Following completion of therapy at 6 months, the median time to rectal MRI was 16 days (range 8-26 days) and the median time to endoscopy was 20.5 days (range 14-28 days). Four additional patients were enrolled and continued on treatment. The results are being being reported without awaiting full enrollment since the decision rule (11 or more responders) is already reached (Table 2).

With a median duration of follow up of 11.8 months (range 6-25), no patients have required chemoradiation and no patients have undergone surgical resection. Since none of the 12 patients who completed 6 months of dostarlimab have required surgery, pCR will not be an evaluable endpoint. No patients demonstrated disease progression or recurrence and all enrolled patients are alive (Table 2, FIGS. 11-12).

The co-primary objective that measures durability of response (sustained cCR at 12 months) is not reported in its finality. To date, four patients have achieved one year of sustained cCR following completion of dostarlimab alone, and no patient on trial has experienced progression of disease or recurrence (FIGS. 11-12).

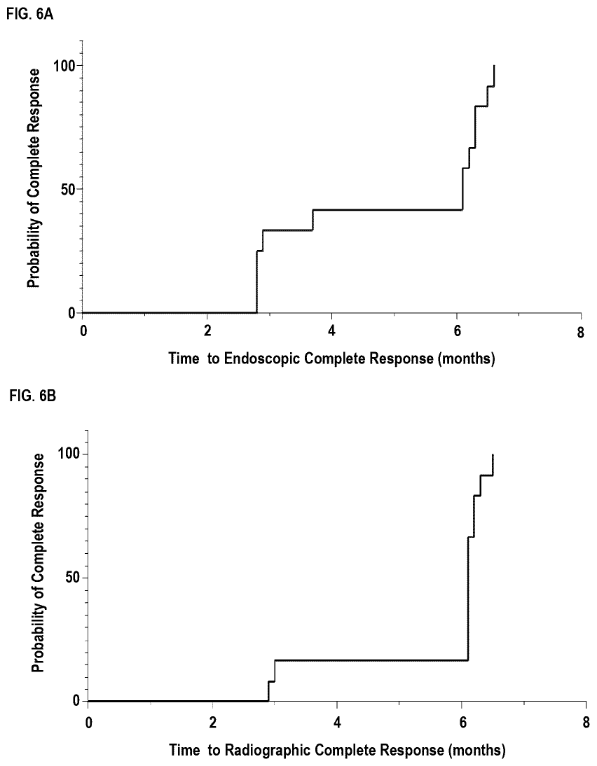

Therapeutic responses were rapid with resolution of symptoms within 6 weeks of starting dosarlimab in 81% of patients (FIG. 9). Five patients achieved an endoscopic CR at the 3-month assessment, while only 2 patients achieved a radiographic CR at the 3-month assessment (FIGS. 6A-6B and FIGS. 11-12).

Safety and Feasibility

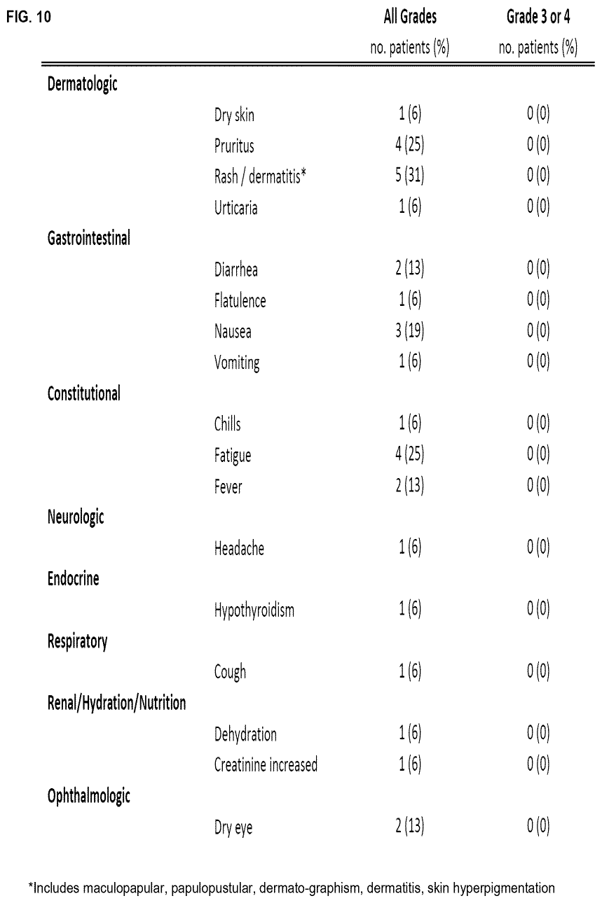

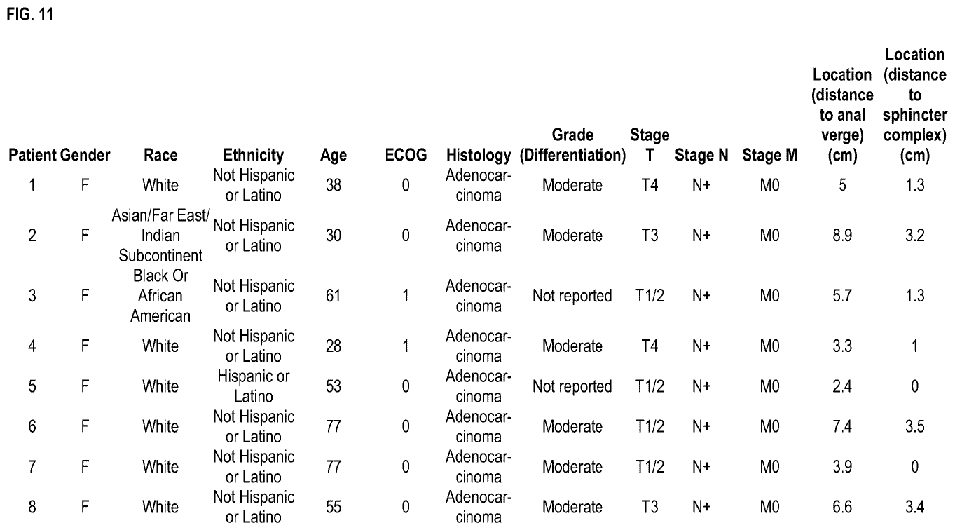

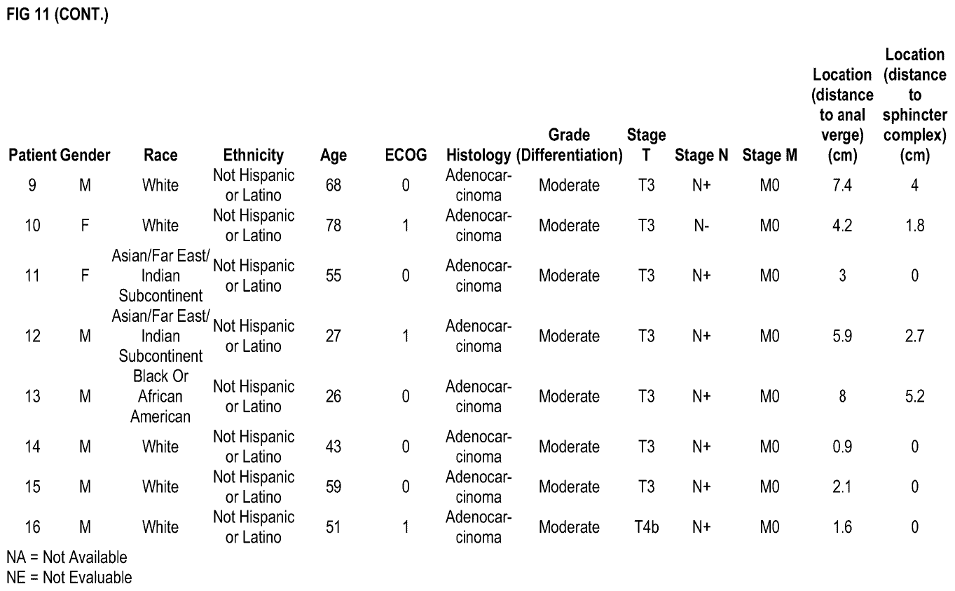

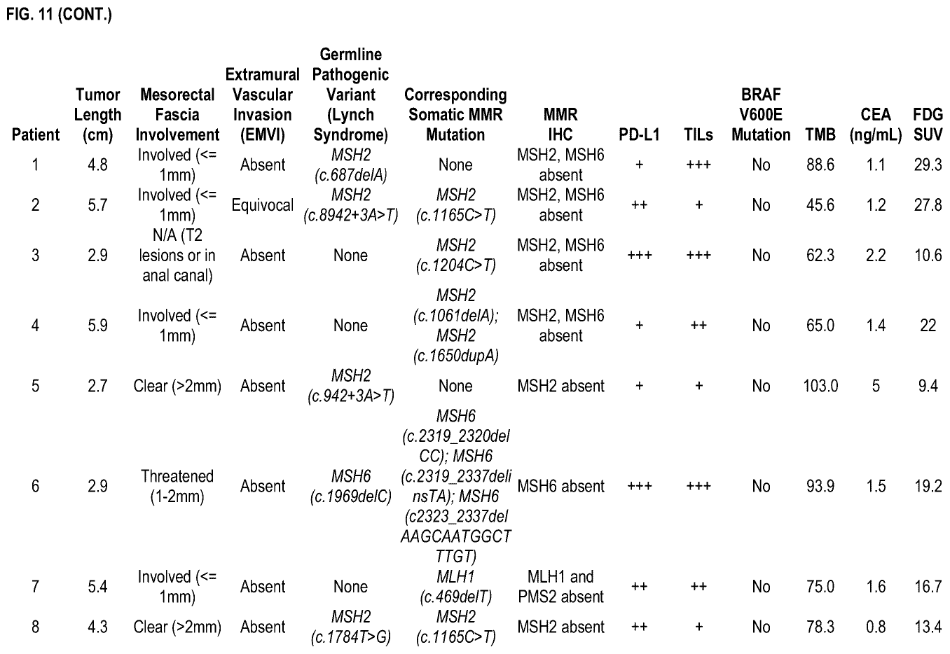

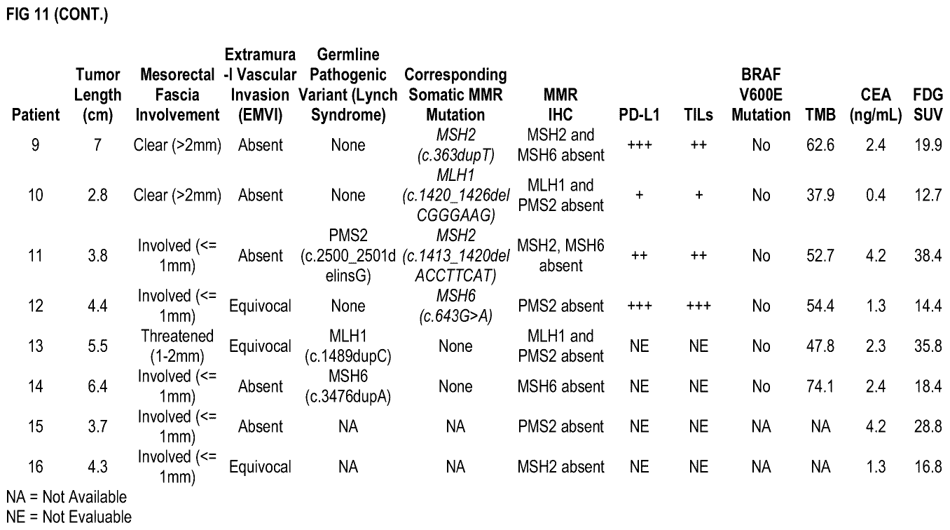

Treatment-related adverse events of any grade occurred in 12 out of 16 patients (75%, 95% CI. 48%-92%). No grade 3 or higher adverse events were observed. The most common grade 1/2 adverse events included rash (31%), pruritus (25%), fatigue (25%), and nausea (19%). Thyroid-function abnormalities occurred in 1 patient (6%) (FIG. 10).

Biomarkers of Longitudinal Response

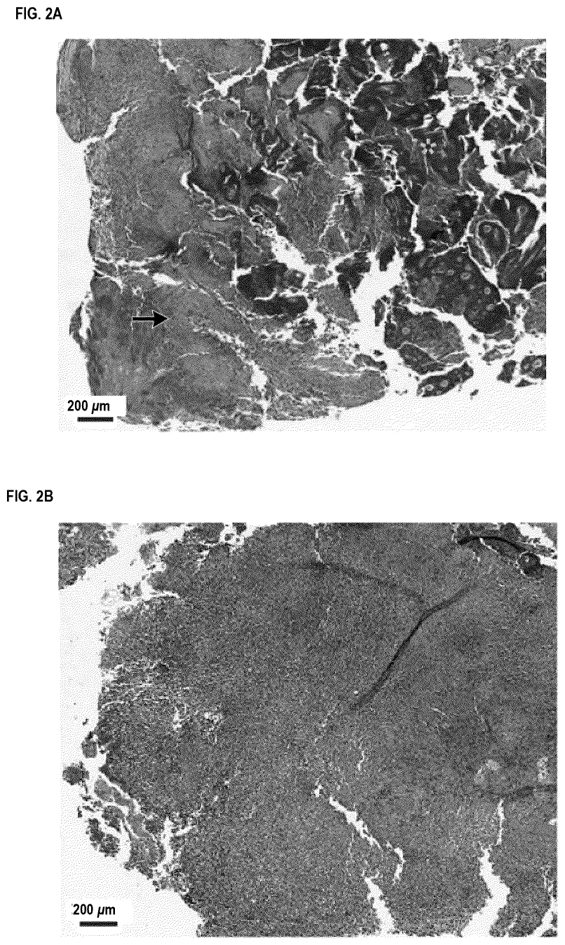

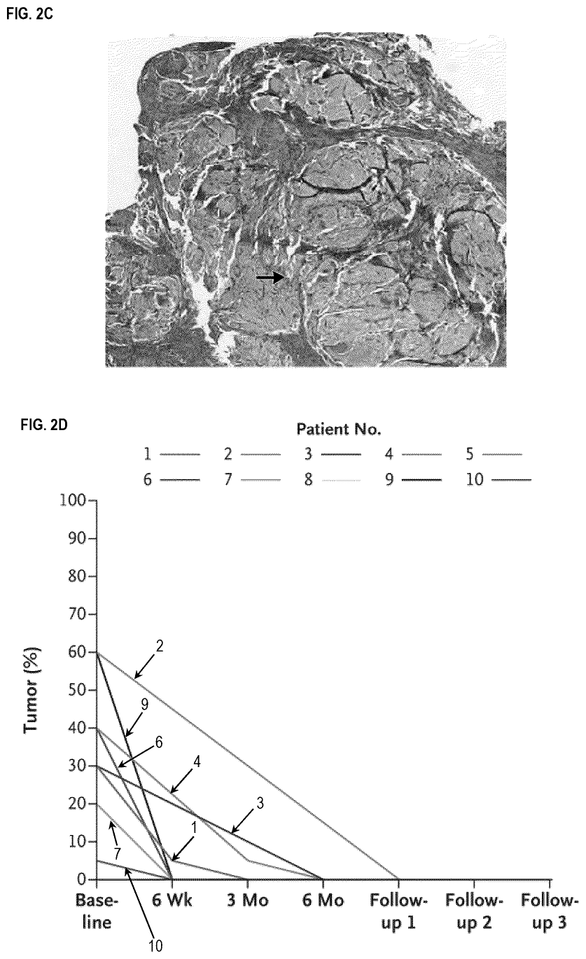

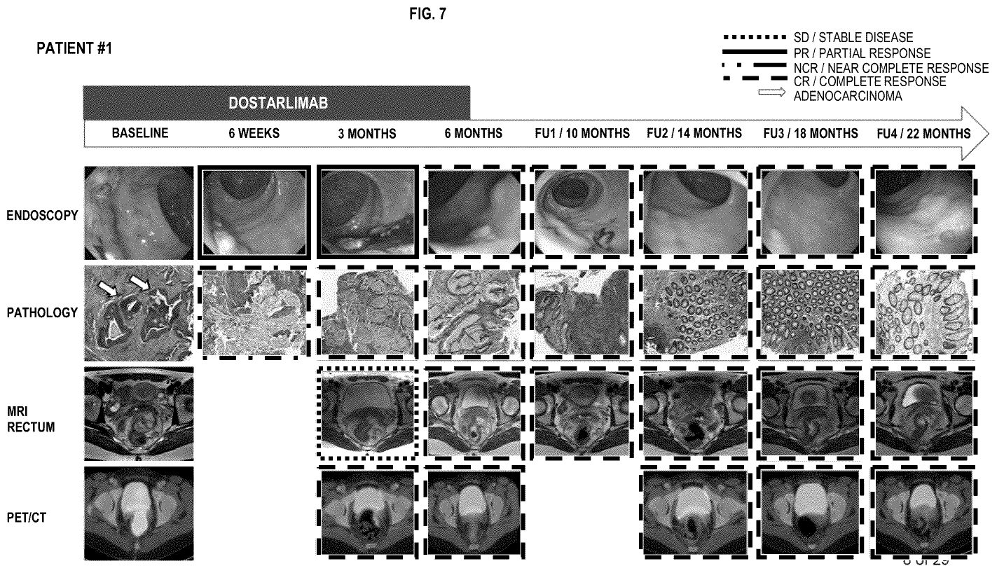

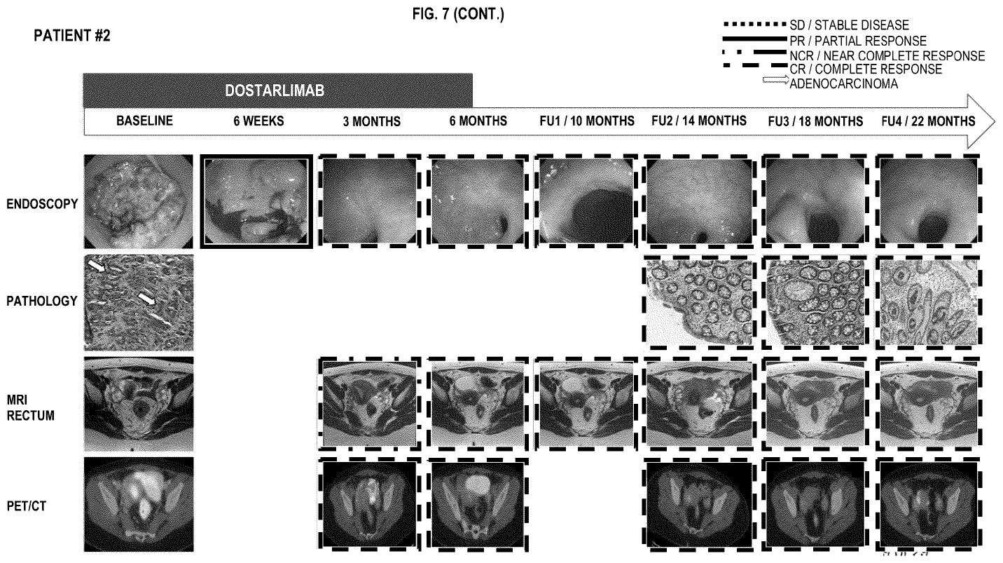

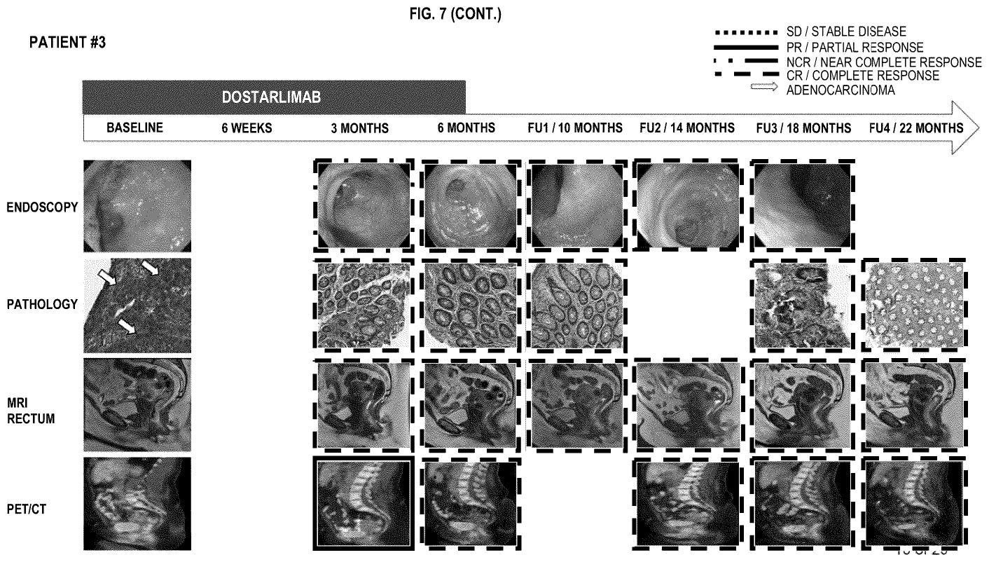

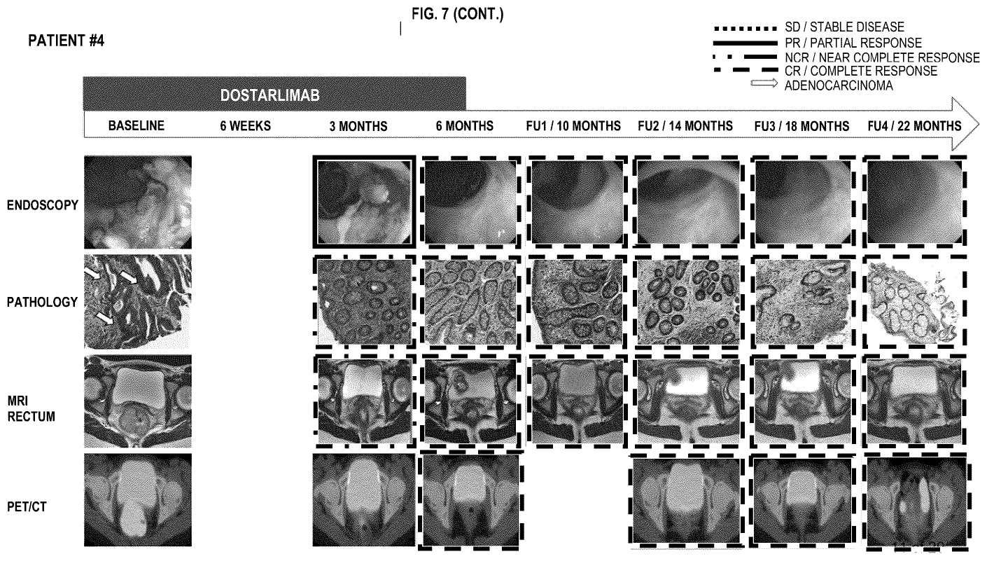

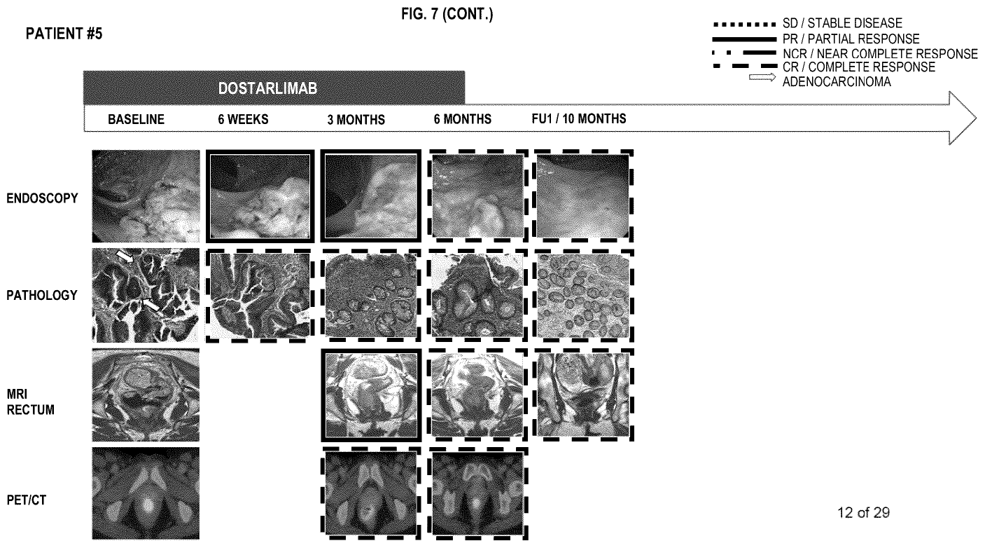

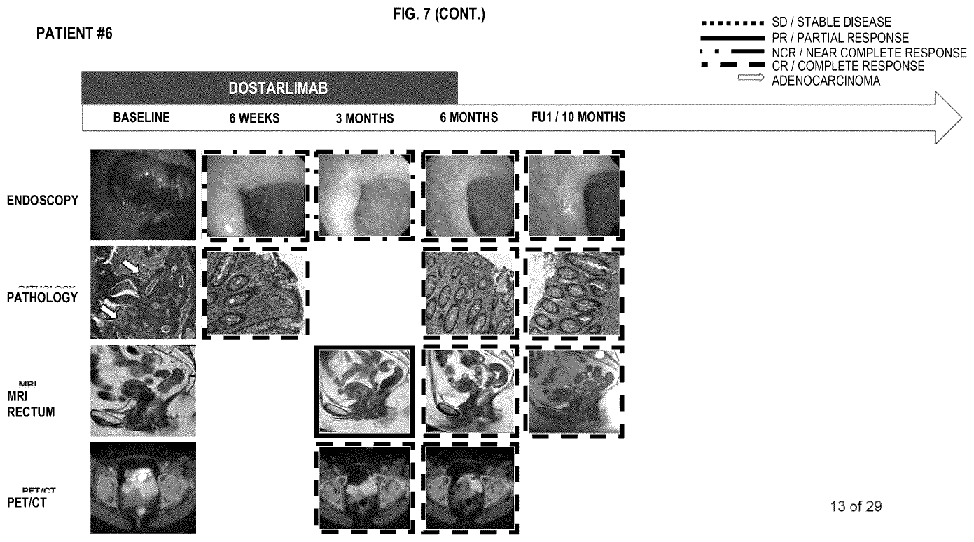

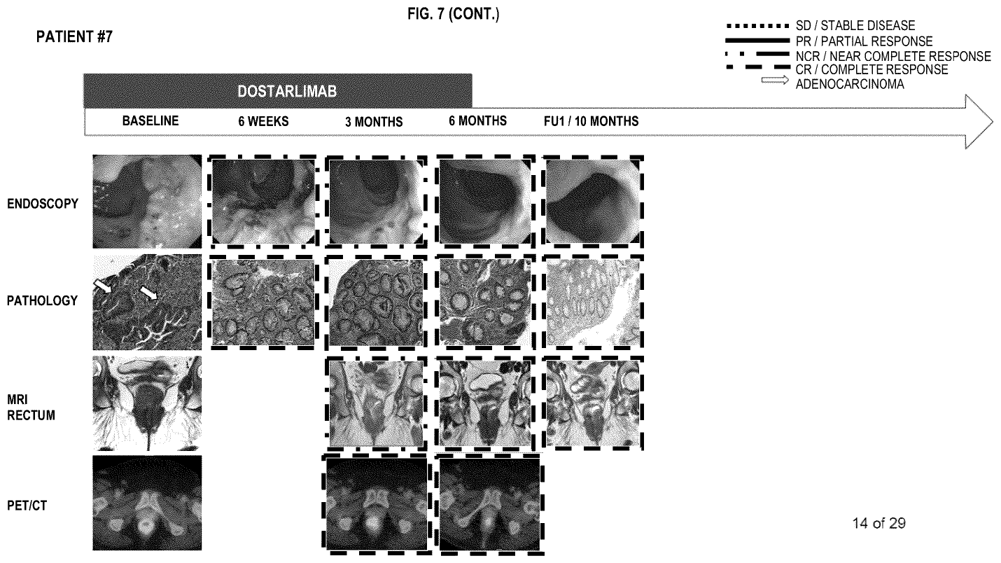

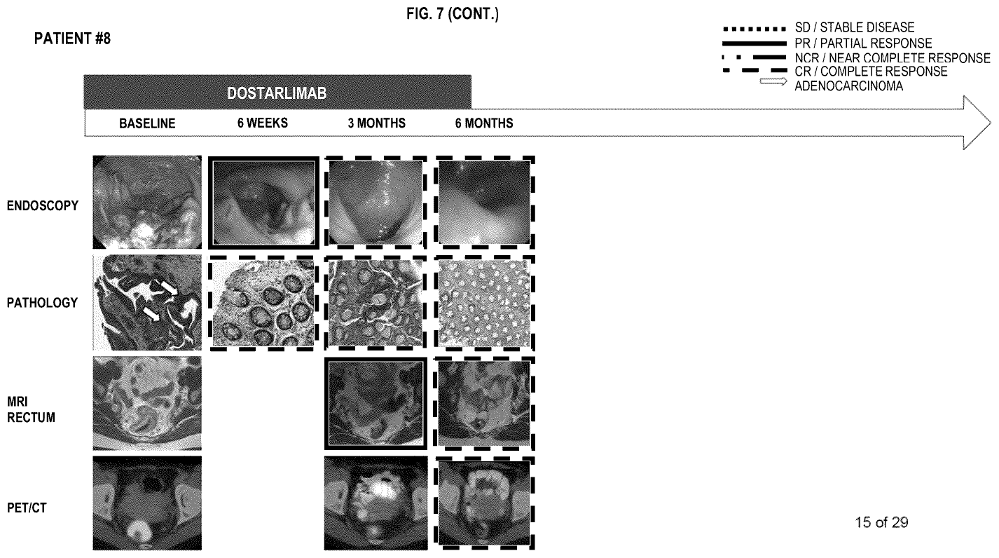

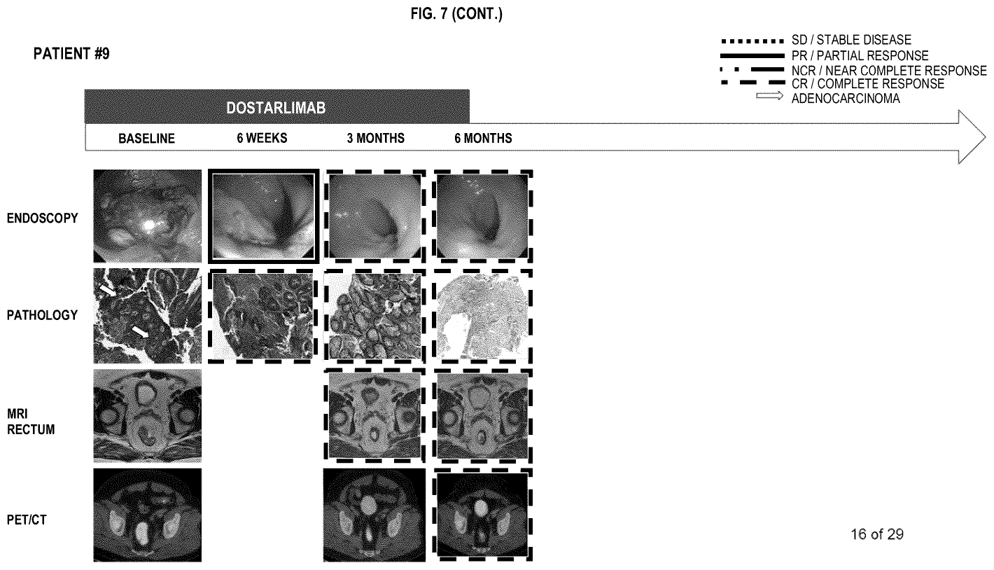

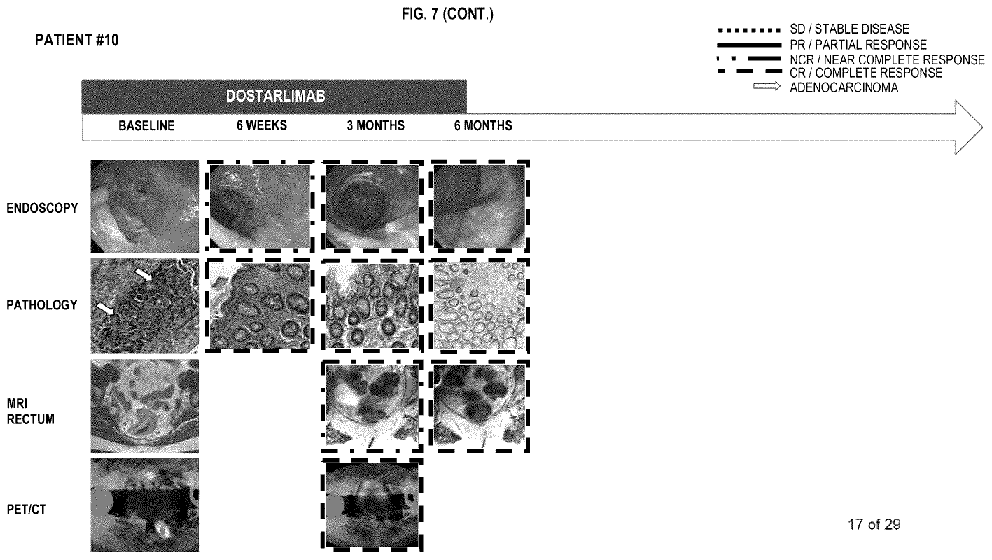

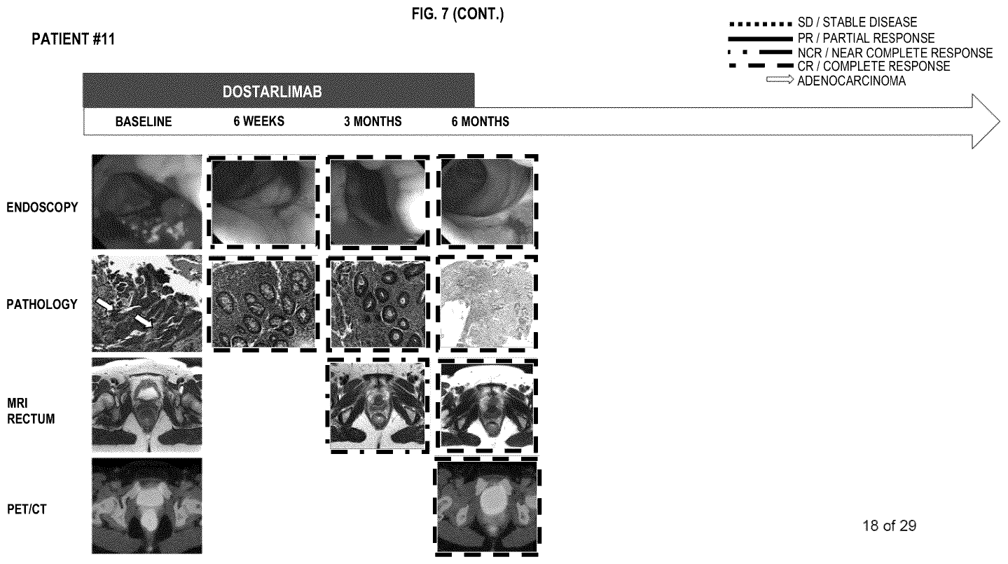

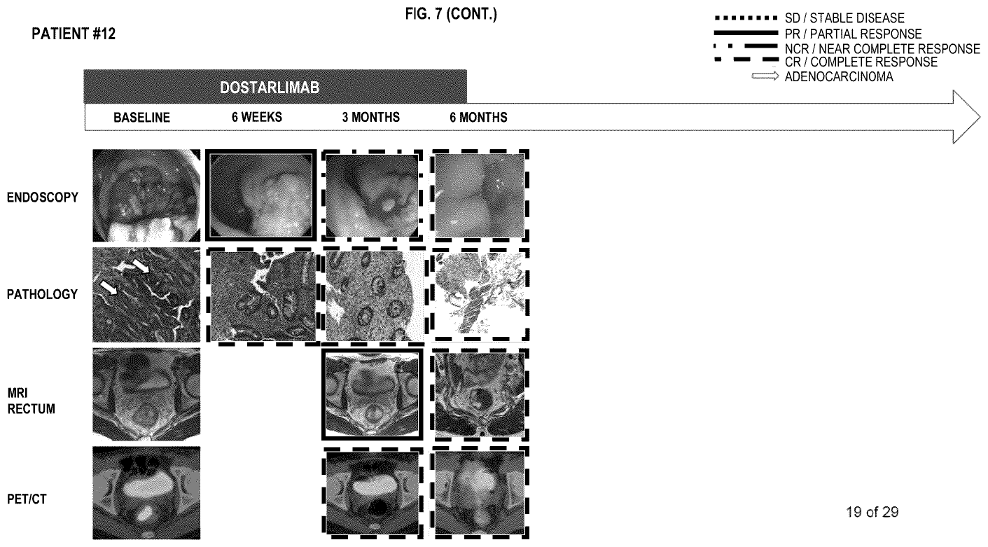

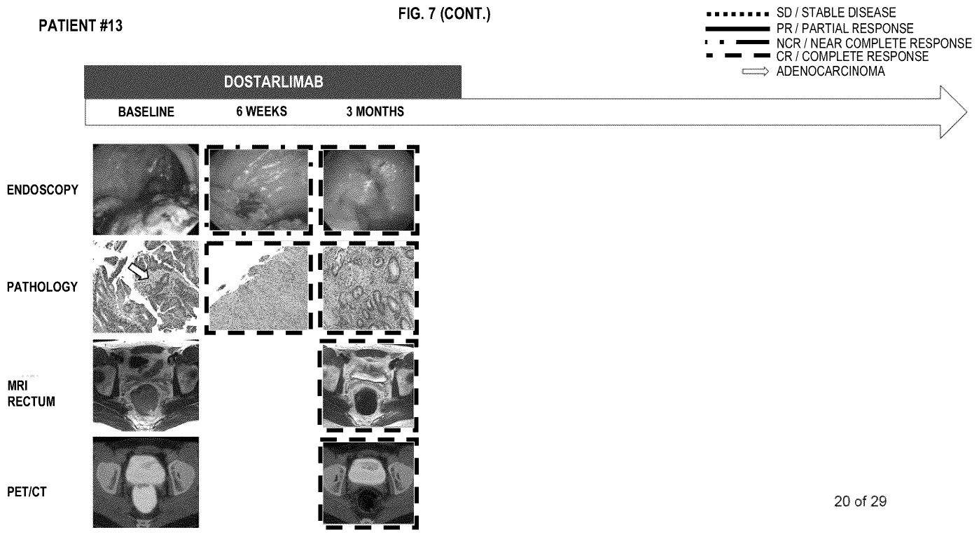

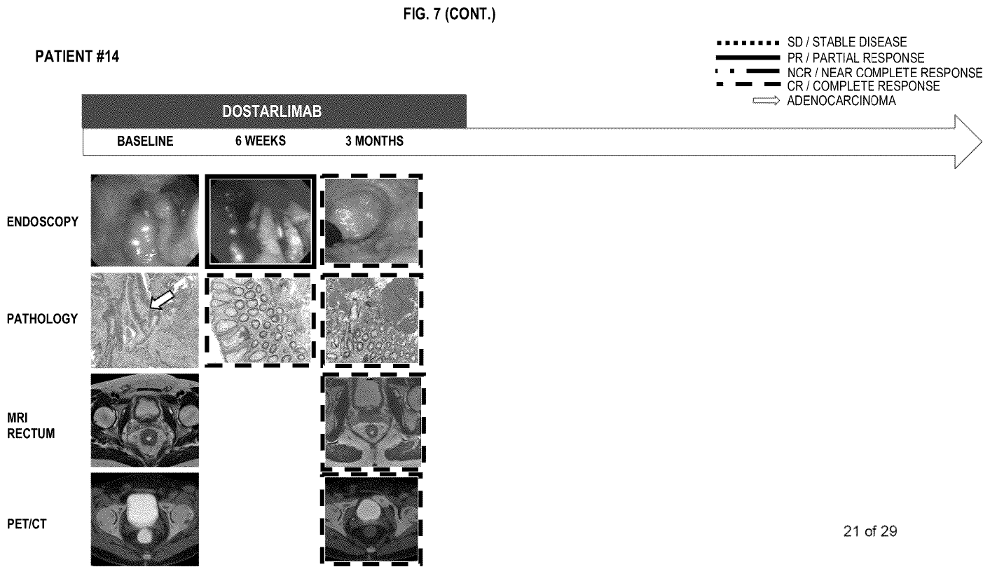

Endoscopic biopsies were performed at baseline and during visual inspection of tumor response at 6 weeks, 3 months, 6 months and then every 4 months. Patients who achieved clinical CR after 6 months of therapy with dostarlimab and had evaluable tissue also had no evidence of tumor on endoscopic biopsy, with the majority showing no evidence of viable tumor as early as 6 weeks into therapy (FIGS. 2A-2D). Longitudinal endoscopic, pathologic, and radiographic data for each patient are depicted in FIG. 7.

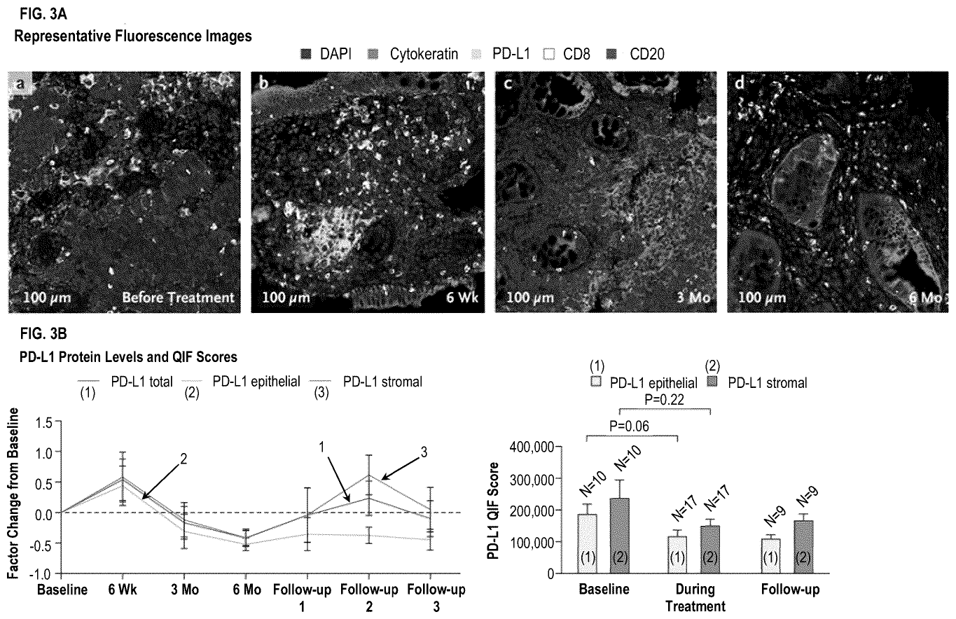

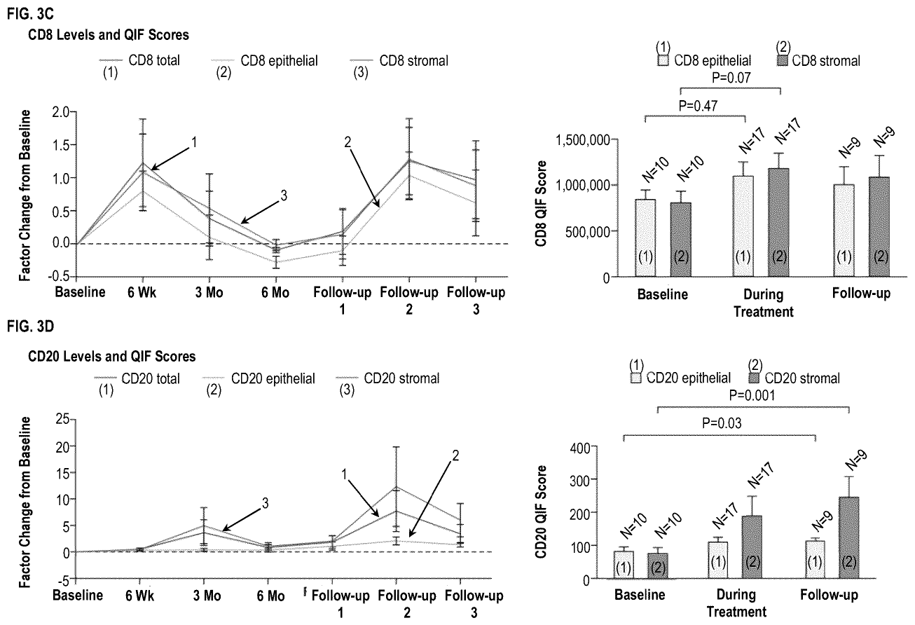

Tumors exhibited variable PD-L1 protein expression with comparable levels on stromal and tumor cells as well as a prominent lymphocytic infiltration enriched for cells expressing CD8 or CD20. PD-L1 protein levels and CD8+ effector tumor infiltrating lymphocytes (TILs) were present at baseline and increased 6 weeks after administration of dostarlimab in both the tumor/epithelial and stromal tissue compartments but decreased transiently from 3 to 6 months while on therapy and then returned to a higher level in the tumor-free rectal mucosa. CD20 positive B-lymphocytes formed nodular aggregates predominantly located in the stromal areas, consistent with tertiary lymphoid structures, that gradually increased in levels after 6 weeks of therapy to levels 6 to 10-fold greater than baseline 6 months after completion of therapy with dostarlimab (FIGS. 3A-3D).

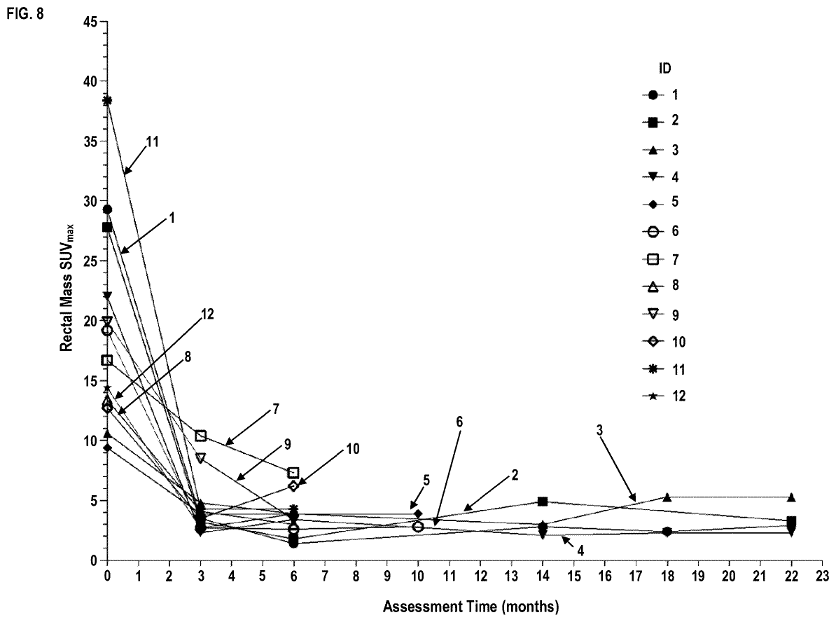

Serial FDG-PET scans at 3 months, 6 months and then every 4 months were captured to further evaluate tumor response to PD-1 blockade and showed a similar evolution of tumor eradication. SUVmax levels were reduced to background levels in all patients as early as 3 months into therapy. All cases that completed 6 months of treatment with dostarlimab showed complete tumor resolution by FDG-PET (FIG. 8).

Neoadjuvant dostarlimab blockade treatment alone in patients with MMRd locally advanced rectal cancer resulted in a clinical complete response, as measured by the combination of DW-MRI, visual endoscopic inspection, and digital rectal examination in all 12 patients with at least 6 months of follow up on this trial. The completeness of these responses is further supported by the absence of residual tumor on serial endoscopic biopsies and resolution of FDG signal on PET scans. At least a near complete response in many of patients after only 3 months of treatment.

The evolution of the local immune response shows a significant initial expansion of PD-L1 and CD8 positive cells, followed by a decrease to below pre-treatment levels in the same time frame that CR is achieved as measured by all modalities. After completion of therapy a repopulation of PD-L1 positive cells is noted, primarily in the stroma, in conjunction with CD8⁺ T-cell expansion and prominent increase in CD20⁺B lymphocytes within tertiary lymphoid structures (data not shown). In melanomas, the co-existence of CD8 and CD20 positive cells and the development of tertiary lymphoid structure has been associated with an improved clinical benefit from checkpoint blockade.⁴⁵ Already four patients of the required thirteen to meet the pre-stated second co-primary endpoint have a sustained clinical CR of more than one year following completion of dostarlimab.

In conclusion, single agent dostarlimab is remarkably effective in MMRd locally advanced rectal cancer, providing a clinical complete response in all tested patients who completed treatment to date. The study also provides a framework for evaluation of highly active anticancer therapies in the neoadjuvant setting, where patients are potentially spared from chemoradiation and surgery while treating the tumor when it is most likely to respond, namely before exposure to other agents that might select cells with a resistant phenotype.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

### REFERENCES

1. Cercek, A. et al. Neoadjuvant chemotherapy first, followed by chemoradiation and then surgery, in the management of locally advanced rectal cancer. JNCCN J. Natl. Compr. Cancer Netw. 12, 513-519 (2014).
2. Chua, Y. J. et al. Neoadjuvant capecitabine and oxaliplatin before chemoradiotherapy and total mesorectal excision in MRI-defined poor-risk rectal cancer: a phase 2 trial. Lancet Oncol. 11, 241-248 (2010).
3. Fernändez-Martos, C. et al. Phase II, randomized study of concomitant chemoradiotherapy followed by surgery and adjuvant capecitabine plus oxaliplatin (CAPOX) compared with induction CAPOX followed by concomitant chemoradiotherapy and surgery in magnetic resonance imaging-defined, locally advanced rectal cancer: Grupo Cäncer de Recto 3 study. J. Clin. Oncol. 28, 859-865 (2010).
4. Zotti, P. et al. Validity and reliability of the MSKCC Bowel Function instrument in a sample of Italian rectal cancer patients. Eur. J. Surg. Oncol. 37, 589-596 (2011).
5. Pollack, J. et al. Late adverse effects of short-course preoperative radiotherapy in rectal cancer. Br. J. Surg. 93, 1519-1525 (2006).
6. Peeters, K. C. M. J. et al. Late Side Effects of Short-Course Preoperative Radiotherapy Combined With Total Mesorectal Excision for Rectal Cancer: Increased Bowel Dysfunction in Irradiated Patients-A Dutch Colorectal Cancer Group Study. J. Clin. Oncol. 23, 6199-206 (2005).
7. Hendren, S. K. et al. Prevalence of male and female sexual dysfunction is high following surgery for rectal cancer. Ann. Surg. 242, 212-223 (2005).
8. Kim, J. Y. et al. A comparative study of voiding and sexual function after total mesorectal excision with autonomic nerve preservation for rectal cancer: laparoscopic versus robotic surgery. Ann. Surg. Oncol. 19, 2485-2493 (2012).
9. Smith, J. J. et al. Assessment of a Watch-and-Wait Strategy for Rectal Cancer in Patients with a Complete Response after Neoadjuvant Therapy. JAMA Oncol. 5, e185896 (2019).
10. Habr-Gama, A. et al. Operative versus nonoperative treatment for stage 0 distal rectal cancer following chemoradiation therapy: Long-term results. in Annals of Surgery vol. 240 711-718 (2004).
11. Maas, M. et al. Wait-and-see policy for clinical complete responders after chemoradiation for rectal cancer. J. Clin. Oncol. 29, 4633-4640 (2011).
12. Park, I. J. et al. Neoadjuvant treatment response as an early response indicator for patients with rectal cancer. J. Clin. Oncol. 30, 1770-1776 (2012).
13. Smith, J. J. & Garcia-Aguilar, J. Advances and challenges in treatment of locally advanced rectal cancer. J. Clin. Oncol. 33, 1797-808 (2015).
14. Robertson, I. et al. Prospective analysis of stoma-related complications. Colorectal Dis. 7, 279-285 (2005).
15. Jonkers, H. A. F. et al. Early complications after stoma formation: a prospective cohort study in 100 patients with 1-year follow-up. Int. J. Colorectal Dis. 27, 1095-1099 (2012).
16. Cercek, A. et al. Mismatch Repair-Deficient Rectal Cancer and Resistance to Neoadjuvant Chemotherapy. Clin. Cancer Res. 26, 3271-3279 (2020).
17. Alex, A. K. et al. Response to Chemotherapy and Prognosis in Metastatic Colorectal Cancer With DNA Deficient Mismatch Repair. Clin. Colorectal Cancer 16, 228-239 (2017).
18. Alatise, O. I. et al. Molecular and phenotypic profiling of colorectal cancer patients in West Africa reveals biological insights. Nat. Commun. 12, (2021).
19. Andre, T. et al. Pembrolizumab versus chemotherapy for microsatellite instability-high/mismatch repair deficient metastatic colorectal cancer: The phase 3 KEYNOTE-177 Study. J. Clin. Oncol. 38, LBA4-LBA4 (2020).
20. Le, D. T. et al. PD-1 blockade in tumors with mismatch-repair deficiency. N. Engl. J. Med. 372, 2509-2520 (2015).
21. Le, D. T. et al. KEYNOTE-164: Pembrolizumab for patients with advanced microsatellite instability high (MSI-H) colorectal cancer. J. Clin. Oncol. 36, 3514-3514 (2018).
22. Overman, M. J. et al. Durable clinical benefit with nivolumab plus ipilimumab in DNA mismatch repair-deficient/microsatellite instability-high metastatic colorectal cancer. J. Clin. Oncol. 36, 773-779 (2018).
23. Seligmann, J. F. & Group, Fo. C. FOxTROT: neoadjuvant FOLFOX chemotherapy with or without panitumumab (Pan) for patients (pts) with locally advanced colon cancer (CC). https://doi.org/10.1200/JCO.2020.38.15_suppl.4013 38, 4013-4013 (2020).
24. Smith, J. J. et al. Organ Preservation in Rectal Adenocarcinoma: a phase II randomized controlled trial evaluating 3-year disease-free survival in patients with locally advanced rectal cancer treated with chemoradiation plus induction or consolidation chemotherapy, and total mesorectal excision or nonoperative management. BMC Cancer 15, (2015).
25. Reese, J. B. et al. Gastrointestinal ostomies and sexual outcomes: a comparison of colorectal cancer patients by ostomy status. Support. Care Cancer 22, 461-468 (2014).
26. Siegel, R. L. et al. Colorectal cancer statistics, 2020. CA. Cancer J. Clin. 70, 145-164 (2020).
27. Schmid, P. et al. Pembrolizumab for Early Triple-Negative Breast Cancer. N. Engl. J. Med. (2020).
28. Forde, P. M. et al. Neoadjuvant PD-1 Blockade in Resectable Lung Cancer. N. Engl. J. Med. 378, 1976-1986 (2018).
29. Martinez Chanza, N. et al. Avelumab as neoadjuvant therapy in patients with urothelial non-metastatic muscle invasive bladder cancer: a multicenter, randomized, non-comparative, phase II study (Oncodistinct 004—AURA trial). BMC Cancer 21, (2021).
30. Amaria, R. N. et al. Neoadjuvant immune checkpoint blockade in high-risk resectable melanoma. Nat. Med. 24, 1649-1654 (2018).
31. Le, D. T. et al. Mismatch repair deficiency predicts response of solid tumors to PD-1 blockade. Science (80-.). 357, 409-413 (2017).
32. Menzies, A. M. et al. Pathological response and survival with neoadjuvant therapy in melanoma: a pooled analysis from the International Neoadjuvant Melanoma Consortium (INMC). Nat. Med. 27, 301-309 (2021).
33. Chalabi, M. et al. Neoadjuvant immunotherapy leads to pathological responses in MMR-proficient and MMR-deficient early-stage colon cancers. Nat. Med. 26, 566-576 (2020).
34. Hu, H. et al. Neoadjuvant PD-1 blockade with toripalimab, with or without celecoxib, in mismatch repair-deficient or microsatellite instability-high, locally advanced, colorectal cancer (PICC): a single-centre, parallel-group, non-comparative, randomised, phase 2 trial. lancet. Gastroenterol. Hepatol. 7, 38-48 (2022).
35. Le, D. T. et al. Programmed death-1 blockade in mismatch repair deficient colorectal cancer. J. Clin. Oncol. 34, 103-103 (2016).
36. Gopalakrishnan, V. et al. Gut microbiome modulates response to anti-PD-1 immunotherapy in melanoma patients. Science (80-.). 359, 97-103 (2018).
37. Davar, D. et al. Fecal microbiota transplant overcomes resistance to anti-PD-1 therapy in melanoma patients. Science 371, 595-602 (2021).
38. Matson, V. et al. The commensal microbiome is associated with anti-PD-1 efficacy in metastatic melanoma patients. Science 359, 104-108 (2018).
39. Cascone, T. et al. Neoadjuvant nivolumab or nivolumab plus ipilimumab in operable non-small cell lung cancer: the phase 2 randomized NEOSTAR trial. Nat. Med. 27, 504-514 (2021).
40. Hamada, T. et al. Fusobacterium nucleatum in Colorectal Cancer Relates to Immune Response Differentially by Tumor Microsatellite Instability Status. Cancer Immunol. Res. 6, 1327-1336 (2018).
41. Cabrita, R. et al. Tertiary lymphoid structures improve immunotherapy and survival in melanoma. Nature 577, 561-565 (2020).

1. A method for treating mismatch repair deficient (MMRd) rectal cancer in a patient in need thereof comprising administering to the patient an effective amount of an anti-PD1 antibody or an antigen binding fragment thereof,
wherein the anti-PD1 antibody or antigen binding fragment comprises a heavy chain immunoglobulin variable domain (V_{H}) and a light chain immunoglobulin variable domain (V_{L}), wherein the V_{H} comprises a V_{H}-CDR1 sequence of SEQ ID NO: 5, a V_{H}-CDR2 sequence of SEQ ID NO: 6, and a V_{H}-CDR3 sequence of SEQ ID NO: 7 and the V_{L} comprises a V_{L}-CDR1 sequence of SEQ ID NO: 8, a V_{L}—CDR2 sequence of SEQ ID NO: 9, and a V_{L}-CDR3 sequence of SEQ ID NO: 10, and
wherein the patient has not received a prior cancer therapy.

2. The method of claim 1, wherein the V_{H} comprises the sequence of SEQ ID NO: 3 and the V_{L} comprises the sequence of SEQ ID NO: 1.

3. The method of claim 1, wherein the anti-PD-1 antibody or antigen binding fragment comprises a heavy chain (HC) amino acid sequence comprising SEQ ID NO: 4 and a light chain (LC) amino acid sequence comprising SEQ ID NO: 2.

4. The method of claim 1, wherein the MMRd rectal cancer is locally advanced rectal cancer.

5. The method of claim 1, wherein the MMRd rectal cancer comprises a deficiency in one or more of MLH1, MSH2, MSH6 and PMS2.

6. The method of claim 1, wherein the MMRd rectal cancer is stage II or stage III.

7. The method of claim 1, wherein the MMRd rectal cancer is node-positive or node-negative.

8. The method of claim 1, wherein the patient is diagnosed with Lynch Syndrome.

9. The method of claim 8, wherein the patient comprises a germline pathogenic variant selected from the group consisting of MSH2 c.687delA, MSH2 c.8942+3A>T, MSH2 c.942+3A>T, MSH6 c.1969delC, MSH2 c.1784T>G, PMS2 c.2500_2501delinsG, MLH1 c.1489dupC, and MSH6 c.3476dupA.

10. The method of claim 1, wherein the patient does not comprise a BRAF V600E mutation and/or comprises tumors having a tumor mutation burden ranging from 30-95 mutations per Megabase.

11. The method of claim 1, wherein the prior cancer therapy is selected from among immunotherapy, chemotherapy, or radiation, optionally wherein the chemotherapy comprises one or more of fluoropyrimidine, leucovorin calcium (folinic acid), fluorouracil, capecitabine, and oxaliplatin.

12. The method of claim 1, wherein the patient exhibits rectal bleeding, constipation, and/or abdominal pain prior to administration of the anti-PD1 antibody or antigen binding fragment.

13. The method of claim 1, wherein the antigen binding fragment is selected from the group consisting of Fab, F(ab′)2, Fab′, scFv, and Fv.

14. The method of claim 1, wherein the anti-PD1 antibody or antigen binding fragment is administered intravenously, intramuscularly, intraperitoneally, subcutaneously, rectally, parenterally, or intradermally.

15. The method of claim 1, wherein the anti-PD1 antibody or antigen binding fragment is administered once per every two-three weeks or once a month.

16. The method of claim 1, wherein the anti-PD1 antibody or antigen binding fragment is administered for at least 6 months.

17. The method of claim 1, wherein the patient exhibits endoscopic complete response (CR) and/or radiographic CR after administration of the anti-PD1 antibody or antigen binding fragment.

18. The method of claim 1, wherein the MMRd rectal cancer has a tumor stage selected from the group consisting of T1, T2, T3 and T4.

19. The method of claim 1, wherein the MMRd rectal cancer comprises a somatic MMR mutation selected from the group consisting of MSH2 c.1165C>T, MSH2 c.1204C>T, MSH2 c.1061delA, MSH2 c.1650dupA, MSH2 c.363dupT, MSH2 c.1413_1420delACCT TCAT, MSH6 c.2319_2320delCC, MSH6 c.2319_2337delinsTA, MSH6 c2323_2337delAAGCAATGGCTTTGT, MSH6 c.643G>A, MLH1 c.469delT, and MLH1 c.1420_1426delCGGGAAG.

20. The method of claim 1, wherein MMR deficiency of the MMRd rectal cancer is determined by immunohistochemistry.


SEQUENCE LISTING (23 sequences)

SEQ ID NO: 1 - protein, 107 aa, synthetic construct
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIK

SEQ ID NO: 2 - protein, 214 aa, synthetic construct
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 116 aa, synthetic construct
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSS

SEQ ID NO: 4 - protein, 443 aa, synthetic construct
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 5 aa, synthetic construct
SYDMS

SEQ ID NO: 6 - protein, 17 aa, synthetic construct
TISGGGSYTYYQDSVKG

SEQ ID NO: 7 - protein, 7 aa, synthetic construct
PYYAMDY

SEQ ID NO: 8 - protein, 11 aa, synthetic construct
KASQDVGTAVA

SEQ ID NO: 9 - protein, 7 aa, synthetic construct
WASTLHT

SEQ ID NO: 10 - protein, 9 aa, synthetic construct
QHYSSYPWT

SEQ ID NO: 11 - DNA, 1329 bp, synthetic construct
  source 1..1329: mol_type = other DNA
gaggtgcagctgttggagtctgggggaggcttggtacagcctggggggtccctgagactctcctgtgcagcctctggattcactttcagtagctatgacatgtcttgggtccgccaggctccagggaaggggctggagtgggtctcaaccattagtggtggtggtagttacacctactatcaagacagtgtgaaggggcggttcaccatctccagagacaattccaagaacacgctgtatctgcaaatgaacagcctgagagccgaggacacggccgtatattactgtgcgtccccttactatgctatggactactgggggcaagggaccacggtcaccgtctcctcagcatccaccaagggcccatcggtcttcccgctagcaccctgctccaggagcacctccgagagcacagccgccctgggctgcctggtcaaggactacttccccgaaccagtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 12 - DNA, 642 bp, synthetic construct
  source 1..642: mol_type = other DNA
gacatccagttgacccagtctccatccttcctgtctgcatatgtaggagacagagtcaccatcacttgcaaggccagtcaggatgtgggtactgctgtagcctggtatcagcaaaaaccagggaaagcccctaagctcctgatctattgggcatccaccctgcacactggggtcccatcaaggttcagcggcagtggatctgggacagaattcactctcacaatcagcagcctgcagcctgaagattttgcaacttattactgtcagcattatagcagctatccgtggacgtttggccaggggaccaagctggagatcaaacggactgtggctgcaccatctgtcttcatcttcccgccatctgatgagcaattgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctcagctcgcccgtcacaaagagcttcaacaggggagagtgt

SEQ ID NO: 13 - protein, 384 aa, Homo sapiens
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 14 - protein, 330 aa, Homo sapiens [sequence omitted]

SEQ ID NO: 15 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 16 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 17 - protein, 452 aa, Homo sapiens
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 18 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 20 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRMAGKPTHVNVSVVMAEVDGTCY

SEQ ID NO: 21 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - DNA, 15 bp, unidentified
  source 1..15: mol_type = unassigned DNA
aagcaatggctttgt

SEQ ID NO: 23 - protein, 6 aa, synthetic construct
HHHHHH